(19) United States
(12) Patent Application Publication
(10) Pub. No.: US 2017/0306402 A1
(43) Pub. Date: Oct. 26, 2017

(54) SYSTEMS AND METHODS FOR CHARACTERIZATION OF MULTIPLE SCLEROSIS

(71) Applicant: Biogen MA Inc., Cambridge, MA (US)

(72) Inventors: Raghavendra Hosur, Cambridge, MA (US); Jadwiga Bienkowska, Cambridge, MA (US)

(21) Appl. No.: 15/510,560

(22) PCT Filed: Sep. 11, 2015
(86) PCT No.: PCT/US15/49780

§ 371 Date: Mar. 10, 2017

Related U.S. Application Data
(60) Provisional application No. 62/050,069, filed on Sep. 12, 2014.

(51) Int. Cl. C12Q 1/68

(52) U.S. Cl. CPC C12Q 1/6883; C12Q 2600/106; C12Q 2600/158

(57) ABSTRACT

Methods and systems to characterize multiple sclerosis (MS) in a subject, e.g., in a subject having a progressive form of MS are disclosed.

### CROSS-REFERENCE TO RELATED APPLICATIONS

This application claims the benefit of U.S. Provisional Application No. 62/050,069, filed Sep. 12, 2014, the entire contents of which are incorporated herein by reference.

### BACKGROUND OF THE INVENTION

Multiple sclerosis (MS) is an inflammatory disease of the brain and spinal cord characterized by recurrent foci of inflammation that lead to destruction of the myelin sheath. Each case of MS displays one of several patterns of presentation and subsequent course. Most commonly, MS first manifests itself as a series of attacks followed by complete or partial remissions as symptoms mysteriously lessen, only to return later after a period of stability. This is called relapsing-remitting (RR) MS. Primary-progressive (PP) MS is characterized by a gradual clinical decline with no distinct remissions, although there may be temporary plateaus or minor relief from symptoms. Secondary-progressive (SP) MS typically begins with a relapsing-remitting course followed by a later primary-progressive course. Rarely, patients may have a progressive-relapsing (PR) course in which the disease takes a progressive path punctuated by acute attacks. PP, SP, and PR are sometimes lumped together and called chronic progressive MS. A few patients experience malignant MS, defined as a swift and relentless decline resulting in significant disability or even death shortly after disease onset.

Currently, no therapy is effective against SPMS. One of the major reasons for this is an increased heterogeneity of disease presentation in SPMS patients. There is a need for improved identification and characterization of MS, including SPMS, patient populations.

### SUMMARY OF THE INVENTION

The invention relates, inter alia, to methods of treating and/or evaluating a subject having multiple sclerosis (MS), e.g., secondary-progressive multiple sclerosis (SPMS) or relapsing-remitting multiple sclerosis (RRMS), or at risk of developing SPMS or RRMS, methods of identifying a subject for treatment with a MS therapy, e.g., SPMS therapy or RRMS therapy, methods of treating or preventing one or more symptoms associated with MS, e.g., SPMS or RRMS, and methods of evaluating or monitoring disease progression in a subject having MS, e.g., SPMS or RRMS, or at risk of developing SPMS or RRMS. Systems for evaluating a subject population having MS, e.g., SPMS or RRMS, and kits for identifying a subject for treatment with an MS therapy and/or clinical outcome are also described herein.

In one aspect, the present invention provides a method of treating and/or evaluating a subject having multiple sclerosis (MS), e.g., secondary-progressive multiple sclerosis (SPMS), or at risk of developing MS, e.g., SPMS. The method includes administering an MS therapy, e.g., an MS therapy described herein, to a subject.

In some embodiments, the subject has MS, e.g., SPMS, or is at risk of developing MS, e.g., SPMS, and the subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated).

In some embodiments, the subject has MS, e.g., SPMS, or is at risk of developing MS, e.g., SPMS, and has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the subject is at risk of developing SPMS and the subject has two, or more (e.g., 2, 3, 4, 5, 6, 7, 8, 9, 10, 11, 12, 13, 14, 15, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the method includes acquiring knowledge and/or evaluating a sample to determine if a subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated), and, based upon that knowledge, administering the subject an MS therapy, e.g., an MS therapy described herein.

In some embodiments, the gene associated with granulocytes is differentially expressed, e.g., up-regulated, by at least about 10%, 20%, 30%, 40%, 50%, 60%, 70%, 80%, 90%, 95%, or 99%, compared to a standard, e.g., an expression level of the gene in granulocytes in a normal subject. In some embodiments, the gene associated with T cells is differentially expressed, e.g., up-regulated, by at least about 10%, 20%, 30%, 40%, 50%, 60%, 70%, 80%, 90%, 95%, or 99%, compared to a standard, e.g., an expression level of the gene in T cells in a normal subject. In some embodiments, the gene associated with erythrocytes is differentially expressed, e.g., up-regulated, by at least about 10%, 20%, 30%, 40%, 50%, 60%, 70%, 80%, 90%, 95%, 99%, or 99.9%, compared to a standard, e.g., an expression level of the gene in erythrocytes in a normal subject.

In certain embodiments, the gene associated with granulocytes is a granulocyte-specific gene. In certain embodiments, the gene associated with T cells is a T cell-specific gene. In certain embodiments, the gene associated with erythrocytes is an erythrocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the two or more genes are differentially expressed, e.g., up-regulated by at least about 0.5, 1, 2, 5, 10, 20, 50, 100, 200, 500, or 1000 fold, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the two or more genes in the pathway described herein, e.g., the CTLA-4 pathway, are differentially expressed, e.g., up-regulated by at least about 0.5, 1, 2, 5, 10, 20, 50, 100, 200, 500, or 1000 fold, or down-regulated by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In certain embodiments, the subject has been treated with an MS therapy, e.g., an alternative MS therapy. In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab).

In some embodiments, the method includes acquiring a sample, e.g., a blood sample, from the subject.

In certain embodiments, the method includes determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes differentially expressed, in the sample.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample. In some embodiments, the sample is obtained from a subject having RRMS and at risk of developing SPMS. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In particular embodiments, the expression levels are determined prior to initiating, during, or after, a treatment in the subject. In some embodiments, the expression levels are determined at the time of diagnosis of the subject with MS, e.g., SPMS or RRMS. In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In some embodiments, the method includes comparing the expression levels of the genes with a standard, e.g., expression levels of the same genes in the same cell type in a normal subject.

In some embodiments, the method includes identifying a subject having one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated), for treatment with an MS therapy, e.g., an MS therapy described herein.

In some embodiments, the method includes identifying a subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of) FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes identifying a subject having two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In certain embodiments, the subject is already receiving an MS therapy, e.g., an MS therapy described herein, and the identification of the differential expression (e.g., up-regulation or down-regulation) of the genes indicates that the subject can receive an alternative MS therapy, e.g., an alternative MS therapy described herein. In certain embodiments, the subject is already receiving an MS therapy, e.g., an MS therapy described herein, and the identification of the differential expression (e.g., up-regulation or down-regulation) of the genes indicates that the subject should stop receiving the MS therapy, or the dose or dosing schedule of the MS therapy should be altered, e.g., reduced or increased.

In some embodiments, the method includes identifying a clinical outcome (e.g., disease severity, disease progression, clinical outcome, or prognosis) of the subject having one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated), wherein the differential expression (e.g., up-regulation) is correlated with or indicative of a clinical score or clinical marker, e.g., a clinical score or clinical marker associated with disease severity, disease progression, clinical outcome, or prognosis, e.g., a clinical score or clinical marker described herein.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In particular embodiments, the method includes selecting an MS therapy, e.g., an MS therapy described herein, for the subject. In some embodiments, the method includes determining a clinical score for the subject, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS). In some embodiments, the method includes determining a clinical marker for the subject, e.g., an MRI marker, e.g., an MRI marker described herein.

In some embodiments, the method includes selecting a subject having MS, e.g., SPMS, or at risk for MS, e.g., SPMS, for treatment with an MS therapy, e.g., an MS therapy described herein, based upon a determination of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated).

In some embodiments, the method includes selecting a subject having MS, e.g., SPMS, or at risk for MS, e.g., SPMS, for treatment with an MS therapy, e.g., an MS therapy described herein, based upon a determination that two or more (e.g., 2, 3, 4, 5, 6, 7, 8, 9, 10, 11, 12, 13, 14, 15, 16, 17, 18, 19, 20, 30, 40, 50, or more) genes described herein are differentially expressed (e.g., up-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes selecting a subject at risk for SPMS, for treatment with an MS therapy, e.g., an MS therapy described herein, based upon a determination that two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In one aspect, the present invention provides a method of treating and/or evaluating a subject having multiple sclerosis (MS), e.g., secondary-progressive multiple sclerosis (SPMS), or at risk of developing MS, e.g., SPMS. The method includes administering an MS therapy, e.g., an MS therapy described herein, to a subject.

In some embodiments, the subject has MS, e.g., SPMS, or is at risk of developing MS, e.g., SPMS, and the subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated).

In some embodiments, the subject has MS, e.g., SPMS, or is at risk of developing MS, e.g., SPMS, and has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the subject is at risk of developing SPMS and the subject has two, or more (e.g., 2, 3, 4, 5, 6, 7, 8, 9, 10, 11, 12, 13, 14, 15, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the method includes acquiring knowledge and/or evaluating a sample to determine if a subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated), and, based upon that knowledge, administering the subject an MS therapy, e.g., an MS therapy described herein.

In some embodiments, the gene associated with granulocytes is differentially expressed, e.g., down-regulated, by at least about 10%, 20%, 30%, 40%, 50%, 60%, 70%, 80%, 90%, 95%, or 99%, compared to a standard, e.g., an expression level of the gene in granulocytes in a normal subject. In some embodiments, the gene associated with T cells is differentially expressed, e.g., down-regulated, by at least about 10%, 20%, 30%, 40%, 50%, 60%, 70%, 80%, 90%, 95%, or 99%, compared to a standard, e.g., an expression level of the gene in T cells in a normal subject. In some embodiments, the gene associated with erythrocytes is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in erythrocytes in a normal subject.

In certain embodiments, the gene associated with granulocytes is a granulocyte-specific gene. In certain embodiments, the gene associated with T cells is a T cell-specific gene. In certain embodiments, the gene associated with erythrocytes is an erythrocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the two or more genes are differentially expressed, e.g., down-regulated by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the two or more genes in the pathway described herein, e.g., the CTLA-4 pathway, are differentially expressed, e.g., up-regulated by at least about 0.5, 1, 2, 5, 10, 20, 50, 100, 200, 500, or 1000 fold, or down-regulated by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In certain embodiments, the subject has been treated with an MS therapy, e.g., an alternative MS therapy. In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab).

In some embodiments, the method includes acquiring a sample, e.g., a blood sample, from the subject.

In certain embodiments, the method includes determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes differentially expressed, in the sample.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, or all) of) FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample. In some embodiments, the sample is obtained from a subject having RRMS and at risk of developing SPMS. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In particular embodiments, the expression levels are determined prior to initiating, during, or after, a treatment in the subject. In some embodiments, the expression levels are determined at the time of diagnosis of the subject with MS, e.g., SPMS or RRMS. In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In some embodiments, the method includes comparing the expression levels of the genes with a standard, e.g., expression levels of the same genes in the same cell type in a normal subject.

In some embodiments, the method includes identifying a subject having one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated), for treatment with an MS therapy, e.g., an MS therapy described herein.

In some embodiments, the method includes identifying a subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., or down-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes identifying a subject having two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In certain embodiments, the subject is already receiving an MS therapy, e.g., an MS therapy described herein, and the identification of the differential expression (e.g., down-regulation or up-regulation) of the genes indicates that the subject can receive an alternative MS therapy, e.g., an alternative MS therapy described herein. In certain embodiments, the subject is already receiving an MS therapy, e.g., an MS therapy described herein, and the identification of the differential expression (e.g., down-regulation or the up-regulation) of the genes indicates that the subject should stop receiving the MS therapy, or the dose or dosing schedule of the MS therapy should be altered, e.g., reduced or increased.

In some embodiments, the method includes identifying a clinical outcome (e.g., disease severity, disease progression, clinical outcome, or prognosis) of the subject having one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated), wherein the differential expression (e.g., down-regulation) is correlated with or indicative of a clinical score or clinical marker, e.g., a clinical score or clinical marker associated with disease severity, disease progression, clinical outcome, or prognosis, e.g., a clinical score or clinical marker described herein.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In particular embodiments, the method includes selecting an MS therapy, e.g., an MS therapy described herein, for the subject. In some embodiments, the method includes determining a clinical score for the subject, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS). In some embodiments, the method includes determining a clinical marker for the subject, e.g., an MRI marker, e.g., an MRI marker described herein.

In some embodiments, the method includes selecting a subject having MS, e.g., SPMS, or at risk for MS, e.g., SPMS, for treatment with an MS therapy, e.g., an MS therapy described herein, based upon a determination of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated). In some embodiments, the method includes selecting a subject having MS, e.g., SPMS, or at risk for MS, e.g., SPMS, for treatment with an MS therapy, e.g., an MS therapy described herein, based upon a determination that two or more (e.g., 2, 3, 4, 5, 6, 7, 8, 9, 10, 11, 12, 13, 14, 15, 16, 17, 18, 19, 20, 30, 40, 50, or more) genes described herein are differentially expressed (e.g., down-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes selecting a subject at risk for SPMS, for treatment with an MS therapy, e.g., an MS therapy described herein, based upon a determination that two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In yet another aspect, the present invention provides a method of identifying a subject having multiple sclerosis (MS), e.g., secondary-progressive multiple sclerosis (SPMS), or at risk of developing SPMS, for treatment with an MS therapy, e.g., an MS therapy described herein.

In some embodiments, the method includes providing a sample, e.g., a blood sample, from a subject having multiple sclerosis (MS), e.g., secondary-progressive multiple sclerosis (SPMS), or at risk of developing MS, e.g., SPMS, determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., up-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; identifying the subject for treatment with an MS therapy, e.g., an MS therapy described herein, on the basis that the subject has two or more of the genes differentially expressed (e.g., down-regulated or up-regulated); and identifying the subject for treatment with an MS therapy, e.g., an MS therapy described herein, on the basis that the subject has two or more of the genes differentially expressed (e.g., down-regulated or up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In certain embodiments, the expression levels are determined prior to initiating, during, or after, a treatment in the subject. In certain embodiments, the expression levels are determined at the time of diagnosis of the subject with MS, e.g., SPMS.

In some embodiments, the method includes comprising comparing the expression levels of the genes with a standard, e.g., expression levels of the same genes in the same cell type in a normal subject.

In some embodiments, the gene associated with granulocytes is differentially expressed, e.g., up-regulated, by at least about 0.5, 1, 2, 5, 10, 20, 50, 100, 200, 500, or 1000 fold, compared to a standard, e.g., an expression level of the gene in granulocytes in a normal subject. In some embodiments, the gene associated with T cells is differentially expressed, e.g., up-regulated, by at least about 0.5, 1, 2, 5, 10, 20, 50, 100, 200, 500, or 1000 fold compared to a standard, e.g., an expression level of the gene in T cells in a normal subject. In some embodiments, the gene associated with erythrocytes is differentially expressed, e.g., up-regulated, by at least about 0.5, 1, 2, 5, 10, 20, 50, 100, 200, 500, or 1000 fold, compared to a standard, e.g., an expression level of the gene in erythrocytes in a normal subject.

In certain embodiments, the gene associated with granulocytes is a granulocyte-specific gene. In certain embodiments, the gene associated with T cells is a T cell-specific gene. In certain embodiments, the gene associated with erythrocytes is an erythrocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes, e.g., two or more genes described herein, differentially expressed (e.g., up-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the two or more genes are differentially expressed, e.g., up-regulated, by at least about 0.5, 1, 2, 5, 10, 20, 50, 100, 200, 500, or 1000 fold, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., the CTLA-4 pathway, differentially expressed (e.g., up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the two or more genes in a pathway described herein, e.g., the CTLA-4 pathway, are differentially expressed, e.g., up-regulated, by at least about 0.5, 1, 2, 5, 10, 20, 50, 100, 200, 500, or 1000 fold, or down-regulated, by at least about 10%, 20%, 30%, 40%, 50%, 60%, 70%, 80%, 90%, 95%, or 99%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab). In certain embodiments, the subject has been treated with an MS therapy, e.g., an alternative MS therapy.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In a further aspect, the present invention provides a method of identifying a subject having multiple sclerosis (MS), e.g., secondary-progressive multiple sclerosis (SPMS), or at risk of developing MS, e.g., SPMS, for treatment with an MS therapy, e.g., an MS therapy described herein.

In some embodiments, the method includes providing a sample, e.g., a blood sample, from a subject having multiple sclerosis (MS), e.g., secondary-progressive multiple sclerosis (SPMS), or at risk of developing MS, e.g., SPMS, determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., down-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; identifying the subject for treatment with an MS therapy, e.g., an MS therapy described herein, on the basis that the subject has two or more of the genes differentially expressed (e.g., down-regulated or up-regulated); and identifying the subject for treatment with an MS therapy, e.g., an MS therapy described herein, on the basis that the subject has two or more of the genes differentially expressed (e.g., down-regulated or up-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In certain embodiments, the expression levels are determined prior to initiating, during, or after, a treatment in the subject. In certain embodiments, the expression levels are determined at the time of diagnosis of the subject with MS, e.g., SPMS.

In some embodiments, the method includes comprising comparing the expression levels of the genes with a standard, e.g., expression levels of the same genes in the same cell type in a normal subject.

In some embodiments, the gene associated with granulocytes is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in granulocytes in a normal subject. In some embodiments, the gene associated with T cells is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in T cells in a normal subject. In some embodiments, the gene associated with erythrocytes is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in erythrocytes in a normal subject.

In certain embodiments, the gene associated with granulocytes is a granulocyte-specific gene. In certain embodiments, the gene associated with T cells is a T cell-specific gene. In certain embodiments, the gene associated with erythrocytes is an erythrocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes, e.g., two or more genes described herein, differentially expressed (e.g., down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the two or more genes are differentially expressed, e.g., down-regulated, by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., the CTLA-4 pathway, differentially expressed (e.g., up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the two or more genes in a pathway described herein, e.g., the CTLA-4 pathway, are differentially expressed, e.g., up-regulated, by at least about 0.5, 1, 2, 5, 10, 20, 50, 100, 200, 500, or 1000 fold, or down-regulated, by at least about 10%, 20%, 30%, 40%, 50%, 60%, 70%, 80%, 90%, 95%, or 99%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab). In certain embodiments, the subject has been treated with an MS therapy, e.g., an alternative MS therapy.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In another aspect, the present invention provides a method of treating or preventing one or more symptoms associated with multiple sclerosis (MS), e.g., secondary-progressive multiple sclerosis (SPMS). The symptom can be a symptom described herein.

In some embodiments, the method includes administering an MS therapy, e.g., an MS therapy described herein, to a subject. In some embodiments, the subject has MS, e.g., SPMS, or at risk of developing MS, e.g., SPMS, and the subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated).

In some embodiments, the subject has MS, e.g., SPMS, or at risk of developing MS, e.g., SPMS, and the subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated) in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., up-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the subject is at risk of developing SPMS, and the subject has two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In yet another aspect, the present invention provides a method of treating or preventing one or more symptoms associated with multiple sclerosis (MS), e.g., secondary-progressive multiple sclerosis (SPMS). The symptom can be a symptom described herein.

In some embodiments, the method includes administering an MS therapy, e.g., an MS therapy described herein, to a subject. In some embodiments, the subject has MS, e.g., SPMS, or at risk of developing MS, e.g., SPMS, and the subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated).

In some embodiments, the subject has MS, e.g., SPMS, or at risk of developing MS, e.g., SPMS, and the subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated) in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., down-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the subject is at risk of developing SPMS, and the subject has two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In another aspect, the present invention provides a method of evaluating or monitoring clinical outcome, e.g., disease severity, disease progression, in a subject having multiple sclerosis (MS), e.g., secondary-progressive multiple sclerosis (SPMS), or at risk of developing MS, e.g., SPMS.

In some embodiments, the method includes providing a sample, e.g., a blood sample, from a subject having multiple sclerosis (MS), e.g., secondary-progressive multiple sclerosis (SPMS), or at risk of developing SPMS, determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, in the sample; comparing the expression levels of the genes with a standard, e.g., expression levels of the same genes in the same cell type in a normal subject; and evaluating or monitoring disease progression on the basis that the subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with erythrocytes differentially expressed (e.g., up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and evaluating or monitoring disease progression on the basis that the subject has two or more of the genes differentially expressed (e.g., up-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; and evaluating or monitoring disease progression on the basis that the subject has two or more of the genes differentially expressed (e.g., up-regulated or down-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In a further aspect, the present invention provides a method of evaluating or monitoring clinical outcome, e.g., disease severity, disease progression, in a subject having multiple sclerosis (MS), e.g., secondary-progressive multiple sclerosis (SPMS), or at risk of developing SPMS.

In some embodiments, the method includes providing a sample, e.g., a blood sample, from a subject having multiple sclerosis (MS), e.g., secondary-progressive multiple sclerosis (SPMS), or at risk of developing SPMS, determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, in the sample; comparing the expression levels of the genes with a standard, e.g., expression levels of the same genes in the same cell type in a normal subject; and evaluating or monitoring disease progression on the basis that the subject has one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with erythrocytes differentially expressed (e.g., down-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and evaluating or monitoring disease progression on the basis that the subject has two or more of the genes differentially expressed (e.g., down-regulated). In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; and evaluating or monitoring disease progression on the basis that the subject has two or more of the genes differentially expressed (e.g., up-regulated or down-regulated). In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In yet another aspect, the present invention provides a method for generating a personalized multiple sclerosis (MS), e.g., secondary-progressive multiple sclerosis (SPMS), treatment report, by obtaining a sample, e.g., a blood sample, from a subject having MS, e.g., SPMS, determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes; and selecting an MS therapy, e.g., an MS therapy described herein, based on the expression levels identified, differential expression (e.g., up-regulation) of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, indicates a first course of treatment; and differential expression (e.g., down-regulation) of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, indicates a second different course of treatment.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; selecting an MS therapy, e.g., an MS therapy described herein, based on the expression levels identified. In some embodiments, differential expression (e.g., up-regulation) of two or more of the genes indicates a first course of treatment. In some embodiments, differential expression (e.g., down-regulation) of two or more of the genes indicates a second different course of treatment. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; selecting an MS therapy, e.g., an MS therapy described herein, based on the expression levels identified. In some embodiments, differential expression (e.g., up-regulation) of two or more of the genes indicates a first course of treatment. In some embodiments, differential expression (e.g., down-regulation) of two or more of the genes indicates a second different course of treatment. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the first course of treatment comprises an MS therapy described herein. In some embodiments, the second course of treatment comprises an MS therapy described herein.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In yet another aspect, the present invention provides a method for generating a personalized multiple sclerosis (MS), e.g., secondary-progressive multiple sclerosis (SPMS), treatment report, by obtaining a sample, e.g., a blood sample, from a subject having MS, e.g., SPMS, determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes; and selecting an MS therapy, e.g., an MS therapy described herein, based on the expression levels identified, differential expression (e.g., down-regulation) of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, indicates a first course of treatment; and differential expression (e.g., up-regulation) of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, indicates a second different course of treatment.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; selecting an MS therapy, e.g., an MS therapy described herein, based on the expression levels identified. In some embodiments, differential expression (e.g., down-regulation) of two or more of the genes indicates a first course of treatment. In some embodiments, differential expression (e.g., up-regulation) of two or more of the genes indicates a second different course of treatment. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; selecting an MS therapy, e.g., an MS therapy described herein, based on the expression levels identified. In some embodiments, differential expression (e.g., down-regulation) of two or more of the genes indicates a first course of treatment. In some embodiments, differential expression (e.g., up-regulation) of two or more of the genes indicates a second different course of treatment. In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the first course of treatment comprises an MS therapy described herein. In some embodiments, the second course of treatment comprises an MS therapy described herein.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In another aspect, the present invention provides a method of determining a gene expression profile for a subject having multiple sclerosis (MS), e.g., secondary-progressive multiple sclerosis (SPMS), or at risk of developing SPMS. In some embodiments, the method includes directly acquiring knowledge of the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, in a sample from a subject having MS, e.g., SPMS, and responsive to a determination of differential expression (e.g., up-regulation or down-regulation) of the genes, one or more of: (1) stratifying a subject population; (2) identifying or selecting the subject as likely or unlikely to respond to an MS therapy, e.g., an MS therapy described herein; (3) selecting an MS therapy, e.g., an MS therapy described herein; (4) treating the subject with an MS therapy, e.g., an MS therapy described herein; or (5) prognosticating the time course and/or severity of the disease in the subject.

In some embodiments, the method includes acquiring knowledge of two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes acquiring knowledge of two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated), and, based upon that knowledge, administering the subject an MS therapy, e.g., an MS therapy described herein. In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In some embodiments, responsive to the direct acquisition of knowledge of the expression levels of the genes, the subject is classified as a candidate to receive an MS therapy, e.g., an MS therapy described herein. In some embodiments, responsive to the direct acquisition of knowledge of the expression levels of the genes, the subject is identified as likely to respond to an MS therapy, e.g., an MS therapy described herein.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In a further aspect, the present invention provides a reaction mixture including: a plurality of detection reagents, or one or more purified or isolated preparations thereof; and a target nucleic acid preparation derived from a sample, e.g., a blood sample, from a subject having multiple sclerosis (MS), e.g., secondary-progressive multiple sclerosis (SPMS), or at risk of developing SPMS.

In some embodiments, the plurality of detection reagents can determine expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the plurality of detection reagents can determine the expression levels of two or more (e.g., 2, 3, 4, 5, 6, 7, 8, 9, 10, 11, or more) genes in a pathway described herein, e.g., the CTLA-4 pathway, in the sample. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

The detection reagent can comprise a probe to measure the expression level of the gene.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In another aspect, the invention provides methods of making a reaction mixture comprising combining a plurality of detection reagents, with a target nucleic acid preparation comprising plurality of target nucleic acid molecules derived from a sample, e.g., from a blood sample, from a subject having multiple sclerosis (MS), e.g., secondary-progressive multiple sclerosis (SPMS), or at risk of developing SPMS.

In some embodiments, the plurality of detection reagents can determine expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the plurality of detection reagents can determine the expression levels of two or more (e.g., 2, 3, 4, 5, 6, 7, 8, 9, 10, 11, or more) genes in a pathway described herein, e.g., the CTLA-4 pathway, in the sample. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

The detection reagent can comprise a probe to measure the expression level of the gene.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In another aspect, the present invention provides a system for evaluating a subject population having multiple sclerosis (MS), e.g., secondary-progressive multiple sclerosis (SPMS), or at risk of developing SPMS, the system comprising at least one processor operatively connected to a memory, the at least one processor has: a first plurality of values for a plurality of subjects having MS, e.g., SPMS, or at risk of developing SPMS, wherein each value is indicative of expression of a gene, e.g., a gene associated with granulocytes, T cells, or erythrocytes; a second plurality of values for the plurality of subjects having MS, e.g., SPMS, or at risk of developing SPMS, wherein each value is indicative of a clinical score for a subject having MS, e.g., SPMS, or at risk of developing SPMS, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS), or wherein each value is indicative of a clinical marker for a subject having MS, or at risk of developing SPMS, e.g., SPMS, e.g., a clinical marker, e.g., an MRI marker, e.g., an MRI marker described herein; and a function that correlates the first plurality of values with the second plurality of values to provide an output of classification of the MS, e.g., SPMS, of the subject population.

In some embodiments, the correlative function determines the joint distribution of the plurality of the subjects in a space of gene expression and clinical score (e.g., a clinical score described herein) or clinical marker (e.g., an MRI marker described herein), e.g., by a method described herein. In certain embodiments, the correlative function determines the joint distribution of the plurality of the subjects in a space of gene expression (X) and clinical score (Y), e.g., by the likelihood maximization problem:

$$Θ = {arg max}_{Θ} P ( Y , X )$$

$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$

where Θ represents the set of parameters used to describe the joint distribution, which includes parameters for the linear regression used to describe P(Y|X,cₘ), parameters for describing the clusters P(X|cₘ) and P(cₘ). In some embodiments, the correlative function uses a regularized Expectation-Maximization algorithm (EM) to learn a sparse set of parameters. In some embodiments, the output indicates an optimal number of clusters for the subject population, e.g., using Bayesian information criterion (BIC).

In yet another aspect, the present invention provides a kit for identifying a subject having multiple sclerosis (MS), e.g., secondary-progressive multiple sclerosis (SPMS), or at risk of developing SPMS, for treatment with an MS therapy, e.g., an MS therapy described herein, and/or for identifying a clinical outcome (e.g., disease severity or disease progression) for a subject having MS, e.g., SPMS.

In some embodiments, the kit includes a product comprising a plurality of agents capable of interacting with a gene expression product of a plurality of genes, wherein the agents detect the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes, in a sample.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the plurality of detection reagents can determine the expression levels of two or more (e.g., 2, 3, 4, 5, 6, 7, 8, 9, 10, 11, or more) genes in a pathway described herein, e.g., the CTLA-4 pathway, in the sample. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In another aspect, the present invention provides an in vitro method of determining if a subject having multiple sclerosis (MS), e.g., secondary-progressive multiple sclerosis (SPMS), is a potential candidate for an MS therapy, e.g., an MS therapy described herein. The method comprises determining the expression levels of one or more (e.g., 1, 2, or 3) of the following: two or more (e.g., 2, 3, 4, 5, 6, 7, 8, 9, 10, 25, 50, 100, 250, 500, or more) genes associated with granulocytes (e.g., myelocytes and/or neutrophils), two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells), or two or more (e.g., 2, 3, 4, 5, 6, 7, 8, 9, 10, 25, 50, 100, 250, 500, or more) genes associated with erythrocytes.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein. In some embodiments, the two or more differentially expressed genes are selected from two or more (e.g., 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

In some embodiments, the method includes determining the expression levels of two or more (e.g., 2, 3, 4, 5, 6, 7, 8, 9, 10, 11, or more) genes in a pathway described herein, e.g., the CTLA-4 pathway. In some embodiments, the subject has RRMS and/or is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In some embodiments, optionally, the method further includes treating the subject with an MS therapy, e.g., an MS therapy described herein, or withholding treatment to the subject of an MS therapy, e.g., an MS therapy described herein.

In certain embodiments, the subject two or more (e.g., 2, 3, 4, 5, 6, 7, 8, 9, 10, 25, 50, 100, 250, 500, or more) genes associated with B cells differentially expressed (e.g., up-regulated or down-regulated). In other embodiments, the subject two or more (e.g., 2, 3, 4, 5, 6, 7, 8, 9, 10, 25, 50, 100, 250, 500, or more) genes associated with dendritic cells differentially expressed (e.g., up-regulated or down-regulated).

In certain embodiments, the expression of two or more (e.g., 2, 3, 4, 5, 6, 7, 8, 9, 10, 25, 50, 100, 250, 500, or more) genes associated with B cells is determined. In other embodiments, the expression of two or more (e.g., 2, 3, 4, 5, 6, 7, 8, 9, 10, 25, 50, 100, 250, 500, or more) genes associated with dendritic cells is determined.

In one aspect, the present invention provides a method of treating and/or evaluating a subject having multiple sclerosis (MS), e.g., relapsing-remitting MS (RRMS), or at risk of developing MS, e.g., secondary-progressive multiple sclerosis (SPMS). The method includes administering an MS therapy, e.g., an MS therapy described herein, to a subject.

In some embodiments, the subject has MS, e.g., RRMS, or is at risk of developing MS, e.g., SPMS, and the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated).

In some embodiments, the subject has MS, e.g., RRMS, or is at risk of developing MS, e.g., SPMS, and has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the subject is at risk of developing SPMS and the subject has two, or more (e.g., 2, 3, 4, 5, 6, 7, 8, 9, 10, 11, 12, 13, 14, 15, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the method includes acquiring knowledge and/or evaluating a sample to determine if a subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), and, based upon that knowledge, administering the subject an MS therapy, e.g., an MS therapy described herein.

In some embodiments, the gene associated with T cells is differentially expressed, e.g., up-regulated, by at least about 10%, 20%, 30%, 40%, 50%, 60%, 70%, 80%, 90%, 95%, or 99%, compared to a standard, e.g., an expression level of the gene in T cells in a normal subject. In some embodiments, the gene associated with granulocytes is differentially expressed, e.g., up-regulated, by at least about 10%, 20%, 30%, 40%, 50%, 60%, 70%, 80%, 90%, 95%, or 99%, compared to a standard, e.g., an expression level of the gene in granulocytes in a normal subject.

In certain embodiments, the gene associated with T cells is a T cell-specific gene. In certain embodiments, the gene associated with granulocytes is a granulocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the two or more genes are differentially expressed, e.g., up-regulated by at least about 0.5, 1, 2, 5, 10, 20, 50, 100, 200, 500, or 1000 fold, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the two or more genes in the pathway described herein, e.g., the CTLA-4 pathway, are differentially expressed, e.g., up-regulated by at least about 0.5, 1, 2, 5, 10, 20, 50, 100, 200, 500, or 1000 fold, or down-regulated by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In certain embodiments, the subject has been treated with an MS therapy, e.g., an alternative MS therapy. In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab).

In some embodiments, the method includes acquiring a sample, e.g., a blood sample, from the subject.

In certain embodiments, the method includes determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample. In some embodiments, the sample is obtained from a subject having RRMS and at risk of developing SPMS. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In particular embodiments, the expression levels are determined prior to initiating, during, or after, a treatment in the subject. In some embodiments, the expression levels are determined at the time of diagnosis of the subject with MS, e.g., SPMS or RRMS. In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In some embodiments, the method includes comparing the expression levels of the genes with a standard, e.g., expression levels of the same genes in the same cell type in a normal subject.

In some embodiments, the method includes identifying a subject having one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), for treatment with an MS therapy, e.g., an MS therapy described herein.

In some embodiments, the method includes identifying a subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes identifying a subject having two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In certain embodiments, the subject is already receiving an MS therapy, e.g., an MS therapy described herein, and the identification of the differential expression (e.g., up-regulation or down-regulation) of the genes indicates that the subject can receive an alternative MS therapy, e.g., an alternative MS therapy described herein. In certain embodiments, the subject is already receiving an MS therapy, e.g., an MS therapy described herein, and the identification of the differential expression (e.g., up-regulation or down-regulation) of the genes indicates that the subject should stop receiving the MS therapy, or the dose or dosing schedule of the MS therapy should be altered, e.g., reduced or increased.

In some embodiments, the method includes identifying a clinical outcome (e.g., disease severity, disease progression, clinical outcome, or prognosis) of the subject having one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated), wherein the differential expression (e.g., up-regulation) is correlated with or indicative of a clinical score or clinical marker, e.g., a clinical score or clinical marker associated with disease severity, disease progression, clinical outcome, or prognosis, e.g., a clinical score or clinical marker described herein.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In particular embodiments, the method includes selecting an MS therapy, e.g., an MS therapy described herein, for the subject. In some embodiments, the method includes determining a clinical score for the subject, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS). In some embodiments, the method includes determining a clinical marker for the subject, e.g., an MRI marker, e.g., an MRI marker described herein.

In some embodiments, the method includes selecting a subject having MS, e.g., RRMS, or at risk for MS, e.g., SPMS, for treatment with an MS therapy, e.g., an MS therapy described herein, based upon a determination of has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated).

In some embodiments, the method includes selecting a subject having MS, e.g., RRMS, or at risk for MS, e.g., SPMS, for treatment with an MS therapy, e.g., an MS therapy described herein, based upon a determination that two or more (e.g., 2, 3, 4, 5, 6, 7, 8, 9, 10, 11, 12, 13, 14, 15, 16, 17, 18, 19, 20, 30, 40, 50, or more) genes described herein are differentially expressed (e.g., up-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes selecting a subject at risk for SPMS, for treatment with an MS therapy, e.g., an MS therapy described herein, based upon a determination that two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In one aspect, the present invention provides a method of treating and/or evaluating a subject having multiple sclerosis (MS), e.g., relapsing-remitting multiple sclerosis (RRMS), or at risk of developing MS, e.g., secondary-progressive multiple sclerosis (SPMS). The method includes administering an MS therapy, e.g., an MS therapy described herein, to a subject.

In some embodiments, the subject has MS, e.g., RRMS, or is at risk of developing MS, e.g., SPMS, and the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated).

In some embodiments, the subject has MS, e.g., RRMS, or is at risk of developing MS, e.g., SPMS, and has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the subject is at risk of developing SPMS and the subject has two, or more (e.g., 2, 3, 4, 5, 6, 7, 8, 9, 10, 11, 12, 13, 14, 15, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1, and, based upon that knowledge, administering the subject an MS therapy, e.g., an MS therapy described herein.

In some embodiments, the gene associated with T cells is differentially expressed, e.g., down-regulated, by at least about 10%, 20%, 30%, 40%, 50%, 60%, 70%, 80%, 90%, 95%, or 99%, compared to a standard, e.g., an expression level of the gene in T cells in a normal subject. In some embodiments, the gene associated with granulocytes is differentially expressed, e.g., down-regulated, by at least about 10%, 20%, 30%, 40%, 50%, 60%, 70%, 80%, 90%, 95%, or 99%, compared to a standard, e.g., an expression level of the gene in granulocytes in a normal subject.

In certain embodiments, the gene associated with T cells is a T cell-specific gene. In certain embodiments, the gene associated with granulocytes is a granulocyte-specific gene. In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the two or more genes are differentially expressed, e.g., down-regulated by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the two or more genes in the pathway described herein, e.g., the CTLA-4 pathway, are differentially expressed, e.g., up-regulated by at least about 0.5, 1, 2, 5, 10, 20, 50, 100, 200, 500, or 1000 fold, or down-regulated by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In certain embodiments, the subject has been treated with an MS therapy, e.g., an alternative MS therapy. In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab).

In some embodiments, the method includes acquiring a sample, e.g., a blood sample, from the subject.

In certain embodiments, the method includes determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample. In some embodiments, the sample is obtained from a subject having RRMS and at risk of developing SPMS. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In particular embodiments, the expression levels are determined prior to initiating, during, or after, a treatment in the subject. In some embodiments, the expression levels are determined at the time of diagnosis of the subject with MS, e.g., SPMS or RRMS. In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In some embodiments, the method includes comparing the expression levels of the genes with a standard, e.g., expression levels of the same genes in the same cell type in a normal subject.

In some embodiments, the method includes identifying a subject having one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), for treatment with an MS therapy, e.g., an MS therapy described herein.

In some embodiments, the method includes identifying a subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., or down-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes identifying a subject having two or more (e.g., 2, 3, 4, 5, 6, 7, 8, 9, 10, 11, or more) genes in a pathway described herein, e.g., a CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In certain embodiments, the subject is already receiving an MS therapy, e.g., an MS therapy described herein, and the identification of the differential expression (e.g., down-regulation or up-regulation) of the genes indicates that the subject can receive an alternative MS therapy, e.g., an alternative MS therapy described herein. In certain embodiments, the subject is already receiving an MS therapy, e.g., an MS therapy described herein, and the identification of the differential expression (e.g., down-regulation or the up-regulation) of the genes indicates that the subject should stop receiving the MS therapy, or the dose or dosing schedule of the MS therapy should be altered, e.g., reduced or increased.

In some embodiments, the method includes identifying a clinical outcome (e.g., disease severity, disease progression, clinical outcome, or prognosis) of the subject having one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated), wherein the differential expression (e.g., down-regulation) is correlated with or indicative of a clinical score or clinical marker, e.g., a clinical score or clinical marker associated with disease severity, disease progression, clinical outcome, or prognosis, e.g., a clinical score or clinical marker described herein.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated), for treatment with an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes identifying a clinical outcome (e.g., disease severity, disease progression, clinical outcome, or prognosis) of the subject having two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In particular embodiments, the method includes selecting an MS therapy, e.g., an MS therapy described herein, for the subject. In some embodiments, the method includes determining a clinical score for the subject, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS). In some embodiments, the method includes determining a clinical marker for the subject, e.g., an MRI marker, e.g., an MRI marker described herein.

In some embodiments, the method includes selecting a subject having MS, e.g., RRMS, or at risk for MS, e.g., SPMS, for treatment with an MS therapy, e.g., an MS therapy described herein, based upon a determination of one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated).

In some embodiments, the method includes selecting a subject having MS, e.g., RRMS, or at risk for MS, e.g., SPMS, for treatment with an MS therapy, e.g., an MS therapy described herein, based upon a determination that two or more (e.g., 2, 3, 4, 5, 6, 7, 8, 9, 10, 11, 12, 13, 14, 15, 16, 17, 18, 19, 20, 30, 40, 50, or more) genes described herein are differentially expressed (e.g., down-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes selecting a subject at risk for SPMS, for treatment with an MS therapy, e.g., an MS therapy described herein, based upon a determination that two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In yet another aspect, the present invention provides a method of identifying a subject having multiple sclerosis (MS), e.g., relapsing-remitting multiple sclerosis (RRMS), or at risk of developing secondary-progressive multiple sclerosis (SPMS), for treatment with an MS therapy, e.g., an MS therapy described herein.

In some embodiments, the method includes providing a sample, e.g., a blood sample, from a subject having multiple sclerosis (MS), e.g., RRMS, or at risk of developing MS, e.g., SPMS, determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., up-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; identifying the subject for treatment with an MS therapy, e.g., an MS therapy described herein, on the basis that the subject has two or more of the genes differentially expressed (e.g., down-regulated or up-regulated); and identifying the subject for treatment with an MS therapy, e.g., an MS therapy described herein, on the basis that the subject has two or more of the genes differentially expressed (e.g., down-regulated or up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In certain embodiments, the expression levels are determined prior to initiating, during, or after, a treatment in the subject. In certain embodiments, the expression levels are determined at the time of diagnosis of the subject with MS, e.g., RRMS.

In some embodiments, the method includes comprising comparing the expression levels of the genes with a standard, e.g., expression levels of the same genes in the same cell type in a normal subject.

In some embodiments, the gene associated with T cells is differentially expressed, e.g., down-regulated, by at least about 0.5, 1, 2, 5, 10, 20, 50, 100, 200, 500, or 1000 fold compared to a standard, e.g., an expression level of the gene in T cells in a normal subject. In some embodiments, the gene associated with granulocytes is differentially expressed, e.g., up-regulated, by at least about 0.5, 1, 2, 5, 10, 20, 50, 100, 200, 500, or 1000 fold, compared to a standard, e.g., an expression level of the gene in granulocytes in a normal subject.

In certain embodiments, the gene associated with T cells is a T cell-specific gene. In certain embodiments, the gene associated with granulocytes is a granulocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes, e.g., two or more genes described herein, differentially expressed (e.g., up-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the two or more genes are differentially expressed, e.g., up-regulated, by at least about 0.5, 1, 2, 5, 10, 20, 50, 100, 200, 500, or 1000 fold, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., the CTLA-4 pathway, differentially expressed (e.g., up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the two or more genes in a pathway described herein, e.g., the CTLA-4 pathway, are differentially expressed, e.g., up-regulated, by at least about 0.5, 1, 2, 5, 10, 20, 50, 100, 200, 500, or 1000 fold, or down-regulated, by at least about 10%, 20%, 30%, 40%, 50%, 60%, 70%, 80%, 90%, 95%, or 99%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab). In certain embodiments, the subject has been treated with an MS therapy, e.g., an alternative MS therapy.

In a further aspect, the present invention provides a method of identifying a subject having multiple sclerosis (MS), e.g., relapsing-remitting multiple sclerosis (RRMS), or at risk of developing MS, e.g., secondary-progressive multiple sclerosis (SPMS), for treatment with an MS therapy, e.g., an MS therapy described herein.

In some embodiments, the method includes providing a sample, e.g., a blood sample, from a subject having MS, e.g., RRMS, or at risk of developing MS, e.g., SPMS, determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., down-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; identifying the subject for treatment with an MS therapy, e.g., an MS therapy described herein, on the basis that the subject has two or more of the genes differentially expressed (e.g., down-regulated or up-regulated); and identifying the subject for treatment with an MS therapy, e.g., an MS therapy described herein, on the basis that the subject has two or more of the genes differentially expressed (e.g., down-regulated or up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the expression levels of the genes are determined by a method described herein, e.g., oligonucleotide array, an immunoassay (e.g., immunohistochemistry, northern blot, or a PCR method (e.g., quantitative RT-PCR). In some embodiments, expression levels of the genes are determined by evaluating the level of protein expression, e.g., by an immunoassay, e.g., by ELISA, immunohistochemistry, immunofluorescence, or western blot.

In certain embodiments, the expression levels are determined prior to initiating, during, or after, a treatment in the subject. In certain embodiments, the expression levels are determined at the time of diagnosis of the subject with MS, e.g., RRMS.

In some embodiments, the method includes comprising comparing the expression levels of the genes with a standard, e.g., expression levels of the same genes in the same cell type in a normal subject.

In some embodiments, the gene associated with T cells is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in T cells in a normal subject. In some embodiments, the gene associated with granulocytes is differentially expressed, e.g., down-regulated, by at least about 10%, 20%, 30%, 40%, 50%, 60%, 70%, 80%, 90%, 95%, 99%, or 99.9%, compared to a standard, e.g., an expression level of the gene in granulocytes in a normal subject. In certain embodiments, the gene associated with T cells is a T cell-specific gene. In certain embodiments, the gene associated with granulocytes is a granulocyte-specific gene.

In some embodiments, the method includes acquiring knowledge and/or evaluating a sample to determine if a subject has two or more (e.g., 2, 3, 4, 5, 6, 7, 8, 9, 10, 11, 12, 13, 14, 15, 16, 17, 18, 19, 20, 30, 40, 50, or more) genes, e.g., two or more genes described herein, differentially expressed (e.g., down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the two or more genes are differentially expressed, e.g., down-regulated, by at least about 10%, 20%, 30%, 40%, 50%, 60%, 70%, 80%, 90%, 95%, 99%, or 99.9%, compared to a standard, e.g., expression levels of the same genes in the same cell type in a normal subject.

In some embodiments, the method includes acquiring knowledge and/or evaluating a sample to determine if the subject has two or more (e.g., 2, 3, 4, 5, 6, 7, 8, 9, 10, 11, or more) genes in a pathway described herein, e.g., the CTLA-4 pathway, differentially expressed (e.g., up-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the two or more genes in a pathway described herein, e.g., the CTLA-4 pathway, are differentially expressed, e.g., up-regulated, by at least about 0.5, 1, 2, 5, 10, 20, 50, 100, 200, 500, or 1000 fold, or down-regulated, by at least about 10%, 20%, 30%, 40%, 50%, 60%, 70%, 80%, 90%, 95%, or 99%, compared to a standard, e.g., expression levels of the same genes in the same cell type in a normal subject.

In particular embodiments, the MS therapy comprises an anti-VLA-4 therapy, e.g., natalizumab. In particular embodiments, the MS therapy comprises an anti-CD25 therapy, e.g., daclizumab. In particular embodiments, the MS therapy comprises an interferon beta, e.g., interferon beta-1a or interferon beta-1b. In particular embodiments, the MS therapy comprises a sphingosine 1-phosphate (SIP) antagonist, e.g., fingolimod. In particular embodiments, the MS therapy comprises glatiramer acetate (GA). In some embodiments, the MS therapy comprises a CTLA-4 antagonist, e.g., an anti-CTLA antibody or a soluble CTLA-4 protein, e.g., a fusion protein comprising the Fc region of the immunoglobulin IgG fused to the extracellular domain of CTLA-4 (e.g., Abatacept). In some embodiments, the MS therapy comprises a CD20 antagonist, e.g., an anti-CD20 antibody (e.g., rituximab). In certain embodiments, the subject has been treated with an MS therapy, e.g., an alternative MS therapy.

In another aspect, the present invention provides a method of treating or preventing one or more symptoms associated with MS, e.g., relapsing-remitting multiple sclerosis (RRMS), or at risk of developing MS, e.g., secondary-progressive multiple sclerosis (SPMS). The symptom can be a symptom described herein.

In some embodiments, the method includes administering an MS therapy, e.g., an MS therapy described herein, to a subject. In some embodiments, the subject has MS, e.g., RRMS, or at risk of developing MS, e.g., secondary-progressive multiple sclerosis (SPMS), and the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated).

In some embodiments, the subject has MS, e.g., RRMS, or at risk of developing MS, e.g., SPMS, and the subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated) in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., up-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the subject is at risk of developing SPMS, and the subject has two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In yet another aspect, the present invention provides a method of treating or preventing one or more symptoms associated with multiple sclerosis (MS), e.g., relapsing-remitting multiple sclerosis (RRMS), or at risk of developing MS, e.g., secondary-progressive multiple sclerosis (SPMS). The symptom can be a symptom described herein.

In some embodiments, the method includes administering an MS therapy, e.g., an MS therapy described herein, to a subject. In some embodiments, the subject has MS, e.g., RRMS, or at risk of developing MS, e.g., SPMS, and the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated).

In some embodiments, the subject has MS, e.g., RRMS, or at risk of developing MS, e.g., SPMS, and the subject has two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., down-regulated) in the sample; comparing the expression levels of the genes with a standard, e.g., expression levels of the same genes in the same cell type in a normal subject; and identifying the subject for treatment with an MS therapy, e.g., an MS therapy described herein, on the basis that the subject has two or more of the genes differentially expressed (e.g., down-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the subject is at risk of developing SPMS, and the subject has two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In another aspect, the present invention provides a method of evaluating or monitoring clinical outcome, e.g., disease severity, disease progression, in a subject having multiple sclerosis (MS), e.g., relapsing-remitting multiple sclerosis (RRMS), or at risk of developing MS, e.g., secondary-progressive multiple sclerosis (SPMS).

In some embodiments, the method includes providing a sample, e.g., a blood sample, from a subject having multiple sclerosis (MS), e.g., RRMS, or at risk of developing SPMS, determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample; comparing the expression levels of the genes with a standard, e.g., expression levels of the same genes in the same cell type in a normal subject; and evaluating or monitoring disease progression on the basis that the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., up-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., up-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and evaluating or monitoring disease progression on the basis that the subject has two or more of the genes differentially expressed (e.g., down-regulated or up-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; and evaluating or monitoring disease progression on the basis that the subject has two or more of the genes differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In a further aspect, the present invention provides a method of evaluating or monitoring clinical outcome, e.g., disease severity, disease progression, in a subject having multiple sclerosis (MS), e.g., relapsing-remitting multiple sclerosis (RRMS), or at risk of developing secondary-progressive multiple sclerosis (SPMS).

In some embodiments, the method includes providing a sample, e.g., a blood sample, from a subject having multiple sclerosis (MS), e.g., RRMS, or at risk of developing SPMS, determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample; comparing the expression levels of the genes with a standard, e.g., expression levels of the same genes in the same cell type in a normal subject; and evaluating or monitoring disease progression on the basis that the subject has one or more (e.g., 1 or 2) of the following: two or more (e.g., 2, 3, 4, 5, 6, 7, 8, 9, 10, 25, 50, 100, 250, 500, or more) genes associated with T cells (e.g., CD8+CD62L-CD45RA+ T cells and/or CD4+CD62L-CD45RA− T cells) differentially expressed (e.g., down-regulated), or two or more (e.g., 2, 3, 4, 5, 6, 7, 8, 9, 10, 25, 50, 100, 250, 500, or more) genes associated with granulocytes (e.g., myelocytes and/or neutrophils) differentially expressed (e.g., down-regulated).

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; comparing the expression levels of the genes with a standard, e.g., expression levels of the same genes in the same cell type in a normal subject; and evaluating or monitoring disease progression on the basis that the subject has two or more of the genes differentially expressed (e.g., down-regulated or up-regulated). In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; and evaluating or monitoring disease progression on the basis that the subject has two or more of the genes differentially expressed (e.g., up-regulated or down-regulated). In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In yet another aspect, the present invention provides a method for generating a personalized multiple sclerosis (MS), e.g., relapsing-remitting multiple sclerosis (RRMS), treatment report, by obtaining a sample, e.g., a blood sample, from a subject having MS, e.g., RRMS, determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes; and selecting an MS therapy, e.g., an MS therapy described herein, based on the expression levels identified, differential expression (e.g., up-regulation) of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, indicates a first course of treatment; and differential expression (e.g., down-regulation) of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, indicates a second different course of treatment.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; selecting an MS therapy, e.g., an MS therapy described herein, based on the expression levels identified. In some embodiments, differential expression (e.g., up-regulation) of two or more of the genes indicates a first course of treatment. In some embodiments, differential expression (e.g., down-regulation) of two or more of the genes indicates a second different course of treatment. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; selecting an MS therapy, e.g., an MS therapy described herein, based on the expression levels identified. In some embodiments, differential expression (e.g., up-regulation) of two or more of the genes indicates a first course of treatment. In some embodiments, differential expression (e.g., down-regulation) of two or more of the genes indicates a second different course of treatment. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are up-regulated.

In some embodiments, the first course of treatment comprises an MS therapy described herein. In some embodiments, the second course of treatment comprises an MS therapy described herein.

In yet another aspect, the present invention provides a method for generating a personalized multiple sclerosis (MS), e.g., relapsing-remitting multiple sclerosis (RRMS), treatment report, by obtaining a sample, e.g., a blood sample, from a subject having MS, e.g., RRMS, determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes; and selecting an MS therapy, e.g., an MS therapy described herein, based on the expression levels identified, differential expression (e.g., down-regulation) of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, indicates a first course of treatment; and differential expression (e.g., up-regulation) of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, indicates a second different course of treatment.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample; selecting an MS therapy, e.g., an MS therapy described herein, based on the expression levels identified. In some embodiments, differential expression (e.g., down-regulation) of two or more of the genes indicates a first course of treatment. In some embodiments, differential expression (e.g., up-regulation) of two or more of the genes indicates a second different course of treatment. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway, in the sample; comparing the expression levels of the genes with a standard, e.g., expression levels of the same genes in the same cell type in a normal subject; selecting an MS therapy, e.g., an MS therapy described herein, based on the expression levels identified. In some embodiments, differential expression (e.g., down-regulation) of two or more of the genes indicates a first course of treatment. In some embodiments, differential expression (e.g., up-regulation) of two or more of the genes indicates a second different course of treatment. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC. In some embodiments, the two or more genes in the CTLA-4 pathway are down-regulated.

In some embodiments, the first course of treatment comprises an MS therapy described herein. In some embodiments, the second course of treatment comprises an MS therapy described herein.

In another aspect, the present invention provides a method of determining a gene expression profile for a subject having multiple sclerosis (MS), e.g., relapsing-remitting multiple sclerosis (RRMS). In some embodiments, the method includes directly acquiring knowledge of the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in a sample from a subject having MS, e.g., RRMS, and responsive to a determination of differential expression (e.g., up-regulation or down-regulation) of the genes, one or more of: (1) stratifying a subject population; (2) identifying or selecting the subject as likely or unlikely to respond to an MS therapy, e.g., an MS therapy described herein; (3) selecting an MS therapy, e.g., an MS therapy described herein; (4) treating the subject with an MS therapy, e.g., an MS therapy described herein; or (5) prognosticating the time course and/or severity of the disease in the subject.

In some embodiments, the method includes acquiring knowledge of two or more (e.g., 2, 3, 4, 5, 6, 7, 8, 9, 10, 11, 12, 13, 14, 15, 16, 17, 18, 19, 20, 30, 40, 50, or more) genes described herein differentially expressed (e.g., up-regulated or down-regulated), and, based upon that knowledge, administering the subject an MS therapy, e.g., an MS therapy described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes acquiring knowledge of two or more (e.g., 2, 3, 4, 5, 6, 7, 8, 9, 10, 11, or more) genes in a pathway described herein, e.g., the CTLA-4 pathway, differentially expressed (e.g., up-regulated or down-regulated), and, based upon that knowledge, administering the subject an MS therapy, e.g., an MS therapy described herein. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In some embodiments, responsive to the direct acquisition of knowledge of the expression levels of the genes, the subject is classified as a candidate to receive an MS therapy, e.g., an MS therapy described herein. In some embodiments, responsive to the direct acquisition of knowledge of the expression levels of the genes, the subject is identified as likely to respond to an MS therapy, e.g., an MS therapy described herein.

In a further aspect, the present invention provides a reaction mixture including: a plurality of detection reagents, or one or more purified or isolated preparations thereof; and a target nucleic acid preparation derived from a sample, e.g., a blood sample, from a subject having multiple sclerosis (MS), e.g., relapsing-remitting multiple sclerosis (RRMS), or at risk of developing MS, e.g., secondary-progressive multiple sclerosis (SPMS).

In some embodiments, the plurality of detection reagents can determine expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the plurality of detection reagents can determine the expression levels of two or more (e.g., 2, 3, 4, 5, 6, 7, 8, 9, 10, 11, or more) genes in a pathway described herein, e.g., the CTLA-4 pathway, in the sample. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

The detection reagent can comprise a probe to measure the expression level of the gene.

In another aspect, the invention provides methods of making a reaction mixture comprising combining a plurality of detection reagents, with a target nucleic acid preparation comprising plurality of target nucleic acid molecules derived from a sample, e.g., from a blood sample, from a subject having multiple sclerosis (MS), e.g., relapsing-remitting multiple sclerosis (RRMS), or at risk of developing MS, e.g., secondary-progressive multiple sclerosis (SPMS).

In some embodiments, the plurality of detection reagents can determine expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the plurality of detection reagents can determine the expression levels of two or more (e.g., 2, 3, 4, 5, 6, 7, 8, 9, 10, 11, or more) genes in a pathway described herein, e.g., the CTLA-4 pathway, in the sample. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

The detection reagent can comprise a probe to measure the expression level of the gene.

In another aspect, the present invention provides a system for evaluating a subject population having multiple sclerosis (MS), e.g., relapsing-remitting multiple sclerosis (RRMS), or at risk of developing MS, e.g., secondary-progressive multiple sclerosis (SPMS), the system comprising at least one processor operatively connected to a memory, the at least one processor has: a first plurality of values for a plurality of subjects having MS, e.g., RRMS, wherein each value is indicative of expression of a gene, e.g., a gene associated with granulocytes or T cells; a second plurality of values for the plurality of subjects having MS, e.g., RRMS, wherein each value is indicative of a clinical score for a subject having MS, e.g., RRMS, e.g., a clinical score associated with disease severity, disease progression, clinical outcome, or prognosis, e.g., a clinical score described herein, e.g., Expanded Disability Status Scale (EDSS), or Multiple Sclerosis Severity Score (MSSS), or wherein each value is indicative of a clinical marker for a subject having MS, e.g., RRMS, e.g., a clinical marker, e.g., an MRI marker, e.g., an MRI marker described herein; and a function that correlates the first plurality of values with the second plurality of values to provide an output of classification of the MS, e.g., RRMS, of the subject population.

In some embodiments, the correlative function determines the joint distribution of the plurality of the subjects in a space of gene expression and clinical score (e.g., a clinical score described herein) or clinical marker (e.g., an MRI marker described herein), e.g., by a method described herein. In certain embodiments, the correlative function determines the joint distribution of the plurality of the subjects in a space of gene expression (X) and clinical score (Y), e.g., by the likelihood maximization problem:

$$Θ = {arg max}_{Θ} P ( Y , X )$$

$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$

where Θ represents the set of parameters used to describe the joint distribution, which includes parameters for the linear regression used to describe P(Y|X,cₘ), parameters for describing the clusters P(X|cₘ) and P(cₘ). In some embodiments, the correlative function uses a regularized Expectation-Maximization algorithm (EM) to learn a sparse set of parameters. In some embodiments, the output indicates an optimal number of clusters for the subject population, e.g., using Bayesian information criterion (BIC).

In yet another aspect, the present invention provides a kit for identifying a subject having multiple sclerosis (MS), e.g., relapsing-remitting multiple sclerosis (RRMS), or at risk of developing secondary-progressive multiple sclerosis (SPMS), for treatment with an MS therapy, e.g., an MS therapy described herein, and/or for identifying a clinical outcome (e.g., disease severity or disease progression) for a subject having MS, e.g., RRMS.

In some embodiments, the kit includes a product comprising a plurality of agents capable of interacting with a gene expression product of a plurality of genes, wherein the agents detect the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in a sample.

In some embodiments, the plurality of detection reagents can determine the expression levels of two or more (e.g., 2, 3, 4, 5, 6, 7, 8, 9, 10, 11, 12, 13, 14, 15, 16, 17, 18, 19, 20, 30, 40, 50, or more) genes described herein in the sample. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the plurality of detection reagents can determine the expression levels of two or more (e.g., 2, 3, 4, 5, 6, 7, 8, 9, 10, 11, or more) genes in a pathway described herein, e.g., the CTLA-4 pathway, in the sample. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In another aspect, the present invention provides an in vitro method of determining if a subject having multiple sclerosis (MS), e.g., relapsing-remitting multiple sclerosis (RRMS), or at risk of developing MS, e.g., secondary-progressive multiple sclerosis (SPMS), is a potential candidate for an MS therapy, e.g., an MS therapy described herein. The method comprises determining the expression levels of one or more (e.g., 1 or 2) of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes.

In some embodiments, the method includes determining the expression levels of two or more (e.g., 2, 3, 4, 5, 6, 7, 8, 9, 10, 11, 12, 13, 14, 15, 16, 17, 18, 19, 20, 30, 40, 50, or more) genes described herein. In some embodiments, the two or more differentially expressed genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

In some embodiments, the method includes determining the expression levels of two or more (e.g., 2, 3, 4, 5, 6, 7, 8, 9, 10, 11, or more) genes in a pathway described herein, e.g., the CTLA-4 pathway. In some embodiments, the subject has RRMS and is at risk of developing SPMS. In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the two or more of the genes are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

In some embodiments, optionally, the method further includes treating the subject with an MS therapy, e.g., an MS therapy described herein, or withholding treatment to the subject of an MS therapy, e.g., an MS therapy described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 depicts an exemplary traditional paradigm of patient stratification. Assumption is that distinct molecular subgroups correspond to different disease severity distributions.

FIG. 2 depicts a mixture of experts toy model. (Left) Univariate case: Y-axis represents the clinical measurement, and X-axis represents the molecular profiles. The colors indicate different dependence relations between Y and X. (Right) Multivariate case: Two groups are determined by the differential expression of g1, g2 and g3 (blue=low and red=high). But the genes g4 and g5, that do not show the same level of differential expression, correlate with the clinical score.

FIG. 3 depicts an exemplary non-negative matrix factorization (NMF) which was used to reduce the dimensionality of the molecular profiles. Number of reduced dimensions (factors) was chosen by maximizing cophenetic correlation.

FIG. 4 depicts an exemplary map of the factors to different cell types using cell-specific expression pattern from D-MAP data. The most differentiating cell types in the two clusters were: T cell, B cell, e-Erythrocyte cells.

FIG. 5 depicts exemplary different dependence between molecular factors and disease severity in the two sub-groups.

FIG. 6 depicts exemplary steps in mixture of experts model for patient stratification.

FIG. 7 depicts exemplary top genes differentially expressed between the SPMS subgroups.

FIG. 8 depicts exemplary different dependence between molecular factors and disease severity in the SPMS subgroups.

FIG. 9A depicts Kaplan Meier plots for time to first relapse, in RRMS_{A} vs. RRMS_{B} groups in the two clinical trials. P-values, corrected for age, baseline EDSS and number of relapses in the prior year are 0.08/0.69 for DEFINE/CONFIRM studies. Lower line: DEFINE-MSA; Upper line: DEFINE-MSB; Lower dots: CONFIRM-MSA; Upper dots: CONFIRM-MSB.

FIG. 9B depicts Kaplan-Meier plots for the time to 12-week confirmed EDSS progression corrected for age, baseline EDSS and number of relapses in prior year, P-values are 0.01/0.94 for DEFINE/CONFIRM studies. Lower line: DEFINE-MSA; Upper line: DEFINE-MSB; Lower dots: CONFIRM-MSA; Upper dots: CONFIRM-MSB.

FIG. 10 depicts prior therapies in RRMSA (left bars) and RRMSB (right bars) subgroups.

FIG. 11A depicts median BIC for 1-54 mixtures of experts.

FIG. 11B shows that in molecular space the clusters are distinguished by the signature derived from top 20 differentially expressed genes that differentiates the subgroups. Upper cluster of dots: MSA; Lower cluster of dots: MSB.

FIG. 11C depicts a plot of the MSSS predicted by the molecular factors with the two defined subgroups represented by red circles and blue triangles. Error bars capture the 95% CI for multiple runs of the algorithm using different initializations.

FIG. 11D depicts density plots for MSSS distribution in the two subgroups.

FIG. 12A shows that median BIC for 3 subgroups is lower than 2 subgroups; however, with this data set the 3 subgroups lead to a highly variable BIC. Thus 2-experts was selected in the final model.

FIG. 12B shows that the molecular signature derived from top 20 differentially expressed genes separates the two clusters, even though the MSSS values are similar. Upper cluster of dots: A; Lower cluster of dots: B.

FIG. 12C depicts a plot of the MSSS predicted by the molecular factors with the two subgroups represented as red and blue. Error bars capture the 95% CI for multiple runs of the algorithm using different initialization.

FIG. 12D depicts density plots for MSSS distribution in the two subgroups.

FIG. 13A depicts the top 20 transcripts that differentiate the subgroups of RRMS_{A} vs RRMS_{B} samples.

FIG. 13B depicts the top 20 transcripts that differentiate the subgroups of SPMS_{A} vs SPMS_{B} samples, few genes are represented by more than one transcript.

FIG. 14A depicts unsupervised clustering of SPMS samples in the space of 25-molecular factors. There is lack of a molecular separation between groups.

FIG. 14B depicts density plots for the MSSS distribution in the two clusters identified in unsupervised clustering.

FIG. 15 depicts ongoing or prior treatments in SPMS clusters: SPMSA (red; right bars) and SPMSB (blue; left bars). Table summarized the names assign to different treatment.

FIG. 16 depicts differential expression of genes according to the pathways. Genes are assigned to pathway as in MetaCore pathway database from Thomson Reuters. Pathway expression is summarized as a geometric mean of genes' expression in the pathway. Significant differences are indicated by FDR corrected p-values: ***=p<0.001, **=p<0.01, *=p<0.05. Left: MSA; Right: MSB.

FIG. 17 depicts differential pathway enrichment analysis from MetaCore (Thomson Reuters). Shown are top 10 pathways enriched in genes differentially expressed between RRMSA and RRMSB (blue bars; bottom bars) and SPMSA and SPMSB (orange bars; upper bars) and most different between the two comparisons. X axis represents significance of the enrichment analysis and the red line is the significance threshold.

FIG. 18A depicts the CTLA-4 pathway signature in the three groups defined by CTLA4-pathway expression signature at baseline. Longitudinal data for CTLA4-pathway signature in baseline-defined groups is shown after 48 and 96 weeks. Numbers indicate samples available at post-baseline time.

FIG. 18B depicts the CTLA-4 pathway signature in the RRMS subgroups RRMS_{A} (red) and RRMS_{B} (blue).

FIG. 18C depicts the CTLA-4 pathway signature in the SPMS subgroups SPMS_{A} and SPMS_{B} compared to healthy controls.

FIG. 19A depicts Cophenetic measure vs. residual for different numbers of NMF molecular factors considered for RRMS data.

FIG. 19B depicts Cophenetic measure vs. residual for different numbers of NMF molecular factors considered for SPMS data.

FIG. 20 depicts a comparison of the traditional paradigm and the mixture of experts approach. A) a Toy model for the traditional approach; B) Toy model for expertMIX. A hypothetical relationship between the molecular variable X and clinical variable Y is depicted. Investigating just the dimension X of molecular markers or just the dimension Y of clinical variables does not reveal any subgroups. The underlying structure is only revealed in the joint X, Y space. When X and Y are multi-variable, as in the real world, deconvoluting the structure by looking at them individually becomes even more challenging.

FIG. 21A depicts the steps in the data processing from probe selection (I) to interpretation of signatures (IV).

FIG. 21B is an outline of the expertMIX algorithm for selection of optimal number of subgroups and features associating with clinical variability.

### DETAILED DESCRIPTION

The invention is based, at least in part, on the discovery that subgroups of multiple sclerosis (MS), e.g., secondary-progressive multiple sclerosis (SPMS) or relapsing-remitting multiple sclerosis (RRMS), patients can be identified by characterizing high or low expression of cell markers specific for, for example, B cells, T cells, and early erythrocyte cells, and that within each subgroup a different molecular signature can reflect a disease score.

### Definitions

As used herein, the term “acquire” or “acquiring” refers to obtaining possession of a physical entity, or a value, e.g., a numerical value, by “directly acquiring” or “indirectly acquiring” the physical entity or the value. “Directly acquiring” means performing a process (e.g., performing an assay or test on a sample or “analyzing a sample” as that term is defined herein) to obtain the physical entity or value. “Indirectly acquiring” refers to receiving the physical entity or value from another party or source (e.g., a third party laboratory that directly acquired the physical entity or value). Directly acquiring a physical entity includes performing a process, e.g., analyzing a sample, that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, “analyzing” a sample includes performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Analyzing a sample can include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

### Subjects

Patients having MS may be identified by criteria establishing a diagnosis of clinically definite MS as defined by the workshop on the diagnosis of MS (Poser et al., Ann. Neurol. 13:227, 1983). Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al., 2001, Recommended diagnostic criteria for multiple sclerosis: guidelines from the International Panel on the Diagnosis of Multiple Sclerosis, Ann Neurol 50:121-127). The McDonald criteria include the use of MRI evidence of CNS impairment over time to be used in diagnosis of MS, in the absence of multiple clinical attacks.

MS may be evaluated in several different ways. Exemplary criteria include: EDSS (Expanded Disability Status Scale), MSSS (Multiple Sclerosis Severity Score), KPS (Karnofsky Performance Scale, and appearance of exacerbations on MRI (Magnetic Resonance Imaging). The EDSS is a means to grade clinical impairment due to MS (Kurtzke, Neurology 33:1444, 1983). Eight functional systems are evaluated for the type and severity of neurologic impairment. Briefly, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). In evaluating effectiveness of MS treatment, a decrease of one full step indicates an effective treatment (Kurtzke, Ann. Neurol. 36:573-79, 1994).

TABLE 1
Expanded Disability Status Scale (Kurtzke JF. Neurology. 1983; 33: 1444-1452)

| EDSS | Description |
|---|---|
| 0.0 | Normal neurological exam |
| 1.0 | No disability, minimal signs on 1 FS |
| 1.5 | No disability, minimal signs on 2 of 7 FS |
| 2.0 | Minimal disability in 1 of 7 FS |
| 2.5 | Minimal disability in 2 FS |
| 3.0 | Moderate disability in 1 FS; or mild disability in 3-4 FS, though fully ambulatory |
| 3.5 | Fully ambulatory but with moderate disability in 1 FS; mild disability in 1 or 2 FS; moderate |
| | disability in 2 FS; or mild disability in 5 FS |
| 4.0 | Fully ambulatory without aid, up and about 12 hours a day despite relatively severe |
| | disability; able to walk without aid 500 meters |
| 4.5 | Fully ambulatory without aid; up and about much of the day; able to work a full day; may |
| | otherwise have some limitations of full activity or require minimal assistance; relatively |
| | severe disability; able to walk without aid 300 meters |
| 5.0 | Ambulatory without aid for about 200 meters; disability impairs full daily activities |
| 5.5 | Ambulatory for 100 meters; disability precludes full daily activities |
| 6.0 | Intermittent or unilateral constant assistance (cane, crutch, or brace) required to walk |
| | 100 meters with or without resting |
| 6.5 | Constant bilateral support (cane, crutch, or braces) required to walk 20 meters without resting |
| 7.0 | Unable to walk beyond 5 meters even with aid, essentially restricted to wheelchair, |
| | wheels self, transfers alone; active in wheelchair about 12 hours a day |
| 7.5 | Unable to take more than a few steps; restricted to wheelchair; may need aid to transfer; |
| | wheels self, but may require motorized chair for full day |
| 8.0 | Essentially restricted to bed, chair, or wheelchair, but may be out of bed much of day; retains |
| | self-care functions; generally effective use of arms |
| 8.5 | Essentially restricted to bed much of day; some effective use of arms; retains some self-care |
| | functions |
| 9.0 | Helpless bed patient; can communicate and eat |
| 9.5 | Unable to communicate effectively or eat/swallow |
| 10.0 | Death due to MS |

FS = functional system(s).

The MSSS is an algorithm that relates scores on the EDSS to the distribution of disability in patients with comparable disease durations (Roxburgh, et al. Neurology 64:1144, 2005). Thus, similar relatively high MSSS numbers will be assigned to patients who accrue moderate disability over a short period of time, or severe disability over a moderate period of time (Pachner, et al. J Neurol Sci 278(1-2): 66, 2009). The MSSS is a powerful method for comparing disease progression using single assessment data, and can be used as a reference table for future disability comparisons (Roxburgh, et al. Neurology 64:1144, 2005).

TABLE 2
The Karnofsky Performance Scale (Karnofsky D, Burchenal J.
Evaluation of Chemotherapeutic Agents. New York, NY:
Columbia University Press; 1949)
Table 2: Karnofsky Performance Scale

| | Percentage | |
|---|---|---|
| Progression | (%) | Description |
| Mild | 100 | Normal; no complaints; no |
| Able to carry on normal | | evidence of disease |
| activity and to work; no | 90 | Able to carry on normal |
| special care needed | | activity; minor signs or |
| | | symptoms of disease |
| | 80 | Normal activity with effort; |
| | | some signs or symptoms of |
| | | disease |
| Moderate | 70 | Cares for self; unable to carry |
| Unable to work; able to live at | | on normal activity or do |
| home and care for most | | active work |
| personal needs; varying | 60 | Requires occasional |
| amount of assistance needed | | assistance; able to care for |
| | | most personal needs |
| | 50 | Requires considerable |
| | | assistance and frequent |
| | | medical care |
| Severe | 40 | Disabled; requires special care |
| Unable to care for self; | | and assistance |
| requires equivalent of | 30 | Severely disabled; hospital |
| institutional or hospital care; | | admission is indicated; death |
| disease may be progressing | | not imminent |
| rapidly | 20 | Very sick; hospital admission |
| | | necessary; active supportive |
| | | treatment necessary |
| | 10 | Moribund; fatal processes |
| | | progressing rapidly |
| | 0 | Death |

Exacerbations on MRI are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are mild, moderate, or severe according to changes in a Neurological Rating Scale (Sipe et al., Neurology 34:1368, 1984). An annual exacerbation rate and proportion of exacerbation-free patients are determined.

Standard Subject:

As used herein, the term “standard subject” or “control subject” refers to a subject who has standard or control level of disease, e.g., multiple sclerosis. In some cases, such a standard or control subject is a “normal subject,” e.g., a healthy subject, e.g., a subject who has not been diagnosed with MS, a subject who currently shows no signs of MS, a subject who has not previously shown signs of MS. In some cases, such standard or control subjects have low levels of disease, e.g., non-clinically definite MS (e.g., clinically isolated syndrome (CIS)), low severity MS (e.g., low EDSS score, low MSSS score), non-progressive MS (e.g., relapsing remitting MS (RRMS)), primary-progressive MS (PPMS), or recently developed secondary-progressive MS.

### Molecular Patient Stratification

MS is an autoimmune disease in which auto-reactive lymphocytes attack the CNS leading to demyelination. MS, e.g., RRMS, can be characterized, e.g., by recurrent relapses as a result of immune-mediated demyelination. Over time, inflammatory events decline in frequency, as the disease evolves into a progressive phase of neurodegeneration (e.g., SPMS). Demographic and clinical characteristics of RRMS and SPMS patients can be similar with the disease duration significantly longer in SPMS population (Confavreux and Vukusic (2006) Brain 129(Pt 3):606-16). The time elapsed from first symptoms to the onset of SPMS can vary widely among patients, reflecting clinical heterogeneity. Once the patients reach the progressive phase of MS, the rate of the disease worsening can be the same regardless of the length of relapsing remitting phase (Leray, et al. (2010) Brain 133(Pt 7):1900-13).

High molecular and/or clinical heterogeneity exist in RRMS and SPMS. Immunologic diversity among RRMS and SPMS patients and differences in therapeutic response can be examined, e.g., by studying whole blood cell or peripheral blood mononuclear cell profiles from MS patients. The molecular characteristics differentiating the subgroups can represent, e.g., lymphocyte activation pathways. These molecular characteristics can be similar in patients treated with disease-modifying therapies or not.

High-throughput profiling technologies, e.g., genetic, transcriptomic, and proteomic approaches, can provide molecular profiles of patient samples. The goal of analyzing molecular profiles is, e.g., to understand to what extent the clinical variability can be explained by the molecular variability. Biomarkers associated with clinical features provide insights into molecular mechanisms underlying the disease and thus contribute to the selection of targeted therapies.

Methods that can be used for molecular patient stratification include, e.g., traditional approach that looks at the molecular profiles independently of the clinical score (Cancer Genome Atlas Network, Nature 490(7418): 61-70, 2012; Chaussabel et al. Immunity 29(1):150-164, 2008; Ottoboni et al. Sci Transl Med 4(153):153ra131, 2012; Perou et al. Proc Natl Acad Sci USA 96(16): 9212-9217, 1999). Sometimes, the initial dimensionality reduction can look for markers associating with disease severity score in the entire cohort and then for subgroups defined by these markers (Wang et al. Lancet 365(9460): 671-679, 2005). After an initial step of dimensionality reduction, an unsupervised clustering can be performed to identify molecularly uniform subgroups of samples. If such sub-groups are identified, next disease or progression scores can be associated with these.

Traditional approach assumes that these molecular subgroups will reflect variability in disease severity and/or progression classes. More recent methods add biologically motivated constraints to the clustering to aid in interpretation. For example, NBS (Network Based Stratification) (Hofree et al. Nat Methods 10(11): 1108-1115, 2013) algorithm identifies patient subgroups that show similar network characteristics and mutational profiles.

Methods that can be used for molecular patient stratification also include, e.g., analyses that look for molecular markers differentiating pre-defined patient or healthy control groups, e.g., investigating whole-blood RNA transcripts differentiating MS patients from healthy controls (Nickles et al. Hum Mol Genet 22(20): 4194-4205, 2013).

The paradigm that has been traditionally applied assumes that molecularly different patient samples will be associated with different clinical measures. Traditional approach identifies molecular sub-groups first and subsequent analyzes disease progression determined that there are indeed significant differences between the groups.

The approach described herein simultaneously discovers molecular subclasses of patients' samples and molecular features that explain the clinical variability. The expectation is, e.g., that molecularly uniform patient samples will represent a more uniform disease severity, prognosis or drug response. The premise of this method is the supposition that in the joint space of molecular and disease scores, there may exist distinct subgroups of patients such that each group is characterized by a different dependence between molecular and disease scores. This approach finds molecular characteristics defining uniform sample subsets and possibly an independent set of characteristics that explain the clinical variability. This approach does not implicitly enforce the constraint that variables defining molecularly distinct subtypes also explain the clinical variability.

### Treatment and MS Therapies

The methods described herein can be used to treat a subject having MS, e.g., SPMS, or at risk of developing SPMS, or to treat or prevent a symptom associated with MS, e.g., SPMS.

As used herein, the term “treating” refers to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment may be administered to a subject who does not exhibit signs of a disease, disorder, and/or condition (e.g., prior to an identifiable symptom) and/or to a subject who exhibits only early signs of a disease, disorder, and/or condition for the purpose of decreasing the risk of developing pathology associated with the disease, disorder, and/or condition.

As used herein, the term “preventing” refers to partially or completely delaying onset of MS, e.g., SPMS; partially or completely delaying onset of one or more symptoms, features, or clinical manifestations of a particular disease, disorder, and/or condition associated with MS, SPMS; partially or completely delaying onset of one or more symptoms, features, or manifestations of a particular disease, disorder, and/or condition prior to an identifiable symptom; partially or completely delaying progression from an latent disease, disorder and/or condition to an active disease, disorder and/or condition; and/or decreasing the risk of developing pathology associated with the disease, disorder, and/or condition.

In certain embodiments, the MS therapy is an anti-VLA-4 therapy. An anti-VLA-4 therapy is a molecule, e.g., a small molecule compound or protein biologic (e.g., an antibody or fragment thereof, such as an antigen-binding fragment thereof) that blocks VLA-4 activity. The molecule that is the anti-VLA-4 therapy is a VLA-4 antagonist. A VLA-4 antagonist includes any compound that inhibits a VLA-4 integrin from binding a ligand and/or receptor. An anti-VLA-4 therapy can be an antibody (e.g., natalizumab (TYSABRI®)) or fragment thereof, or a soluble form of a ligand. Soluble forms of the ligand proteins for a4 integrins include soluble VCAM-I or fibronectin peptides, VCAM-I fusion proteins, or bifunctional VCAM-I/Ig fusion proteins. For example, a soluble form of a VLA-4 ligand or a fragment thereof may be administered to bind to VLA-4, and in some instances, compete for a VLA-4 binding site on cells, thereby leading to effects similar to the administration of antagonists such as anti-VLA-4 antibodies. For example, soluble VLA-4 integrin mutants that bind VLA-4 ligand but do not elicit integrin-dependent signaling are suitable for use in the described methods. Such mutants can act as competitive inhibitors of wild type integrin protein and are considered “antagonists.” Other suitable antagonists are “small molecules.”

“Small molecules” are agents that mimic the action of peptides to disrupt VLA-4/ligand interactions by, for instance, binding VLA-4 and blocking interaction with a VLA-4 ligand (e.g., VCAM-I or fibronectin), or by binding a VLA-4 ligand and preventing the ligand from interacting with VLA-4. One exemplary small molecule is an oligosaccharide that mimics the binding domain of a VLA-4 ligand (e.g., fibronectin or VCAM-I) and binds the ligand-binding domain of VLA-4. (See, Devlin et al., Science 249: 400-406 (1990); Scott and Smith, Science 249:386-390 (1990); and U.S. Pat. No. 4,833,092 (Geysen), all incorporated herein by reference). A “small molecule” may be chemical compound, e.g., an organic compound, or a small peptide, or a larger peptide-containing organic compound or non-peptidic organic compound. A “small molecule” is not intended to encompass an antibody or antibody fragment. Although the molecular weight of small molecules is generally less than 2000 Daltons, this figure is not intended as an absolute upper limit on molecular weight.

In certain embodiments, the MS therapy is an agent that modulates (e.g., inhibits or activates) a pathway described herein, e.g., the CTLA-4 pathway. For example, the agents can modulate (e.g., inhibits or activates) one or more components (e.g., genes or gene products) in the pathway, e.g., the CTLA-4 pathway, directly or indirectly. In some embodiments, the agent is an antibody, e.g., an anti-CTLA-4 antibody, e.g., ipilimumab. In some embodiments, the agent is a fusion protein, e.g., an Fc region of an immunoglobulin fused to the extracellular domain of CTLA-4, e.g., abatacept. In some embodiments, the agent is a small molecule, e.g., a small molecule that modulates (e.g., inhibits or activates) CTLA-4.

In some embodiments, the MS therapy comprises a CD20 antagonist, e.g., an anti-CD20 antibody (e.g., rituximab).

Non-limiting examples of additional or alternative MS therapies for use in accordance with the present invention include, but are not limited to: fumaric acid salts, such as dimethyl fumarate; Sphingosine 1-phosphate (S1P)-antagonists, such as the S1B-blocking antibody Sphingomab; interferons, such as human interferon beta-1a (e.g., AVONEX® or Rebif®)) and interferon β-1b (BETASERON® human interferon (3 substituted at position 17; Berlex/Chiron); glatiramer acetate (also termed Copolymer 1, Cop-1; COPAXONE® Teva Pharmaceutical Industries, Inc.); an antibody or a fragment thereof (such as an antigen-binding fragment thereof), such as an anti-CD20 antibody, e.g., Rituxan® (rituximab), or an antibody or fragment thereof that competes with or binds an overlapping epitope with rituximab; mixtoxantrone (NOVANTRONE®, Lederle); a chemotherapeutic agent, such as clabribine (LEUSTATIN®), azathioprine (IMURAN®), cyclophosphamide (CYTOXAN®), cyclosporine-A, methotrexate, 4-aminopyridine, and tizanidine; a corticosteroid, such as methylprednisolone (MEDRONE®, Pfizer), or prednisone; CTLA4 Ig; alemtuzumab (MabCAMPATH®) or daclizumab (an antibody that binds CD25); statins; and TNF antagonists.

Glatiramer acetate is a protein formed from a random chain of amino acids (glutamic acid, lysine, alanine and tyrosine (hence GLATiramer)). Glatiramer acetate can be synthesized in solution from these amino acids at a ratio of approximately 5 parts alanine to 3 parts lysine, 1.5 parts glutamic acid and 1 part tyrosine using N-carboxyamino acid anhydrides.

Non-limiting examples of additional or alternative MS therapies for use in accordance with the present invention include, but are not limited to: antibodies or antagonists of other human cytokines or growth factors, for example, TNF, LT, IL-1, IL-2, IL-6, IL-7, IL-8, IL-12 IL-15, IL-16, IL-18, EMAP-11, GM-CSF, FGF, and PDGF. Still other exemplary agents include antibodies to cell surface molecules such as CD2, CD3, CD4, CD8, CD25, CD28, CD30, CD40, CD45, CD69, CD80, CD86, CD90 or their ligands. For example, daclizubmab is an anti-CD25 antibody that may ameliorate multiple sclerosis.

Still other exemplary antibodies include antibodies that provide an activity of an agent described herein, such as an antibody that engages an interferon receptor, e.g., an interferon beta receptor. Typically, in implementations in which the agent includes an antibody, it binds to a target protein other than VLA-4 or other than an α4 integrin, or at least an epitope on VLA-4 other than one recognized by natalizumab.

Still other exemplary agents include: FK506, rapamycin, mycophenolate mofetil, leflunomide, non-steroidal anti-inflammatory drugs (NSAIDs), for example, phosphodiesterase inhibitors, adenosine agonists, antithrombotic agents, complement inhibitors, adrenergic agents, agents that interfere with signaling by proinflammatory cytokines as described herein, IL-1β converting enzyme inhibitors (e.g., Vx740), anti-P7s, PSGL, TACE inhibitors, T-cell signaling inhibitors such as kinase inhibitors, metalloproteinase inhibitors, sulfasalazine, azathloprine, 6-mercaptopurines, angiotensin converting enzyme inhibitors, soluble cytokine receptors and derivatives thereof, as described herein, anti-inflammatory cytokines (e.g. IL-4, IL-10, IL-13 and TGF).

In some embodiments, an agent may be used to treat one or more symptoms or side effects of MS. Such agents include, e.g., amantadine, baclofen, papaverine, meclizine, hydroxyzine, sulfamethoxazole, ciprofloxacin, docusate, pemoline, dantrolene, desmopressin, dexamethasone, tolterodine, phenytoin, oxybutynin, bisacodyl, venlafaxine, amitriptyline, methenamine, clonazepam, isoniazid, vardenafil, nitrofurantoin, psyllium hydrophilic mucilloid, alprostadil, gabapentin, nortriptyline, paroxetine, propantheline bromide, modafinil, fluoxetine, phenazopyridine, methylprednisolone, carbamazepine, imipramine, diazepam, sildenafil, bupropion, and sertraline. Many agents that are small molecules have a molecular weight between 150 and 5000 Daltons.

Examples of TNF antagonists include chimeric, humanized, human or in vitro generated antibodies (or antigen-binding fragments thereof) to TNF (e.g., human TNF α), such as D2E7, (human TNFα antibody, U.S. Pat. No. 6,258,562; BASF), CDP-571/CDP-870/BAY-10-3356 (humanized anti-TNFα antibody; Celltech/Pharmacia), cA2 (chimeric anti-TNFα antibody; REMICADE™, Centocor); anti-TNF antibody fragments (e.g., CPD870); soluble fragments of the TNF receptors, e.g., p55 or p75 human TNF receptors or derivatives thereof, e.g., 75 kd TNFR-IgG (75 kD TNF receptor-IgG fusion protein, ENBREL™; Immunex; see, e.g., Arthritis & Rheumatism 37:S295, 1994; J. Invest. Med. 44:235A, 1996), p55 kdTNFR-IgG (55 kD TNF receptor-IgG fusion protein (LENERCEPT™)); enzyme antagonists, e.g., TNFα converting enzyme (TACE) inhibitors (e.g., an alpha-sulfonyl hydroxamic acid derivative, WO 01/55112, and N-hydroxyformamide TACE inhibitor GW 3333, -005, or -022); and TNF-bp/s-TNFR (soluble TNF binding protein; see, e.g., Arthritis & Rheumatism 39:S284, 1996; Amer. J. Physiol.—Heart and Circulatory Physiology 268:37-42, 1995).

In one implementation, two or more agents are provided as a co-formulation. For example, in some embodiments, an anti-VLA-4 therapy and a second agent are provided as a co-formulation, and the co-formulation is administered to the subject. It is further possible, e.g., at least 24 hours before or after administering the co-formulation, to administer separately one dose of a first agent formulation and then one dose of a formulation containing a second agent. In another implementation, the first agent and the second agent are provided as separate formulations, and the step of administering includes sequentially administering the first agent and the second agent. The sequential administrations can be provided on the same day (e.g., within one hour of one another or at least 3, 6, or 12 hours apart) or on different days.

The first agent and the second agent each can be administered as a plurality of doses separately in time. The first agent and the second agent are typically each administered according to a regimen. The regimen for one or both may have a regular periodicity. The regimen for the first agent can have a different periodicity from the regimen for the second agent, e.g., one can be administered more frequently than the other. In one implementation, one of the first agent and the second agent is administered once weekly and the other once monthly. In another implementation, one of the first agent and the second agent is administered continuously, e.g., over a period of more than 30 minutes but less than 1, 2, 4, or 12 hours, and the other is administered as a bolus. The first agent and the second agent can be administered by any appropriate method, e.g., subcutaneously, intramuscularly, or intravenously.

In some embodiments, each of the first agent and the second agent is administered at the same dose as each is prescribed for monotherapy. In other embodiments, the first agent is administered at a dosage that is equal to or less than an amount required for efficacy if administered alone. Likewise, the second agent can be administered at a dosage that is equal to or less than an amount required for efficacy if administered alone.

### Pathways

Exemplary pathways enriched in the differentially expressed genes from the MS (e.g., RRMS, SPMS, or both) stratification include, one or more (e.g., 1, 2, 3, 4, 5, 6, 7, 8, 9, 10, 11, 12, 13, 14, 15, 16, or all) of the pathways described herein.

In some embodiments, the pathway is selected from, e.g., complement pathway (e.g., disruption in thrombotic microangiopathy), NFAT pathway (e.g., in immune response), CD16 signaling (e.g., in NK cells), inhibitory PD-1 signaling (e.g., in T cells), CD28 signaling, CTLA-4 pathway (e.g., in regulation of T cell function), IL-7 signaling (e.g., in T lymphocytes), EGFR signaling pathway, ICOS pathway (e.g., in T-helper cell), or thrombopoietin-regulated cell processes. In certain embodiments, the pathway is a CTLA-4 pathway, e.g., in regulation of T cell function by CTLA-4. In some embodiments, the differentially expressed gene or genes are selected from, e.g., Zap70, PIK3R2, CD247, AKT2, NFATC2, LAT, LCK, FYN, CD3D, LAT, and TRAC.

Other exemplary pathways include, but are not limited to, HDAC and calcium/calmodulin-dependent kinase (CaMK) pathway, putataive SUMO-1 pathway, putative pathways for stimulation of fat cell differentiation by Bisphenol A, and alpha-1A adrenergic receptor-dependent inhibition of PI3K pathway, canonical WNT signaling pathway, AKT signaling pathway, growth hormone signaling pathway via PI3K/AKT and MAPK cascades, TCR and CD28 co-stimulation pathway, insulin signaling pathway (generic cascades), GM-CSF signaling, glucocorticoid receptor signaling, HGF signaling pathway, and BCR pathway.

### CTLA-4 Signaling Pathway

CTLA-4 (Cytotoxic T-Lymphocyte Antigen-4 or CD152) is a member of the immunoglobin superfamily. It is a type 1 transmembrane glycoprotein having 223 amino acids and a molecular weight of 41-43 kDa. CTLA-4 has an extracellular, single IgV-like domain containing the B7-1 (CD80)/B7-2 (CD86) ligand-binding site. CTLA-4 is a negative regulator of T cell activation and is capable of terminating early events in the receptor-mediated signaling cascade. The costimulatory CTLA-4 pathway can modulate, e.g., inhibit, T cell activation.

Expression of CTLA-4 is dependent on TCR stimulation by the antigens and CD28-B7 engagement (Teft et al. Annu Rev Immunol. 24: 65-97, 2005; Jain et al. Proc Natl Acad Sci USA. 107(4):1524-1528, 2010). Costimulation involves an integration of activating signals and inhibitory signals (e.g., from CD28 and CTLA-4 molecules, respectively) with TCR signals to determine the outcome of a T cell's encounter with antigen (Bour-Jordan et al. Immunol Rev. 241(1): 180-205, 2011; Ahmed et al. Immunology. 126(3):363-377, 2009). Stimulation of TCR can be triggered by Major Histocompatibility Complex (MHC) molecules on antigen presenting cells (APCs). CTLA-4 pathway is involved in various functions, e.g., T cell development, homeostasis, activation, acquisition of effector's functions, and apoptosis.

Accumulation of CD28 can induce expression of CTLA-4 and stabilize CTLA-4 mRNA. CTLA-4 can bind to B7-1/B7-2 homodimers forming a linear zipper-like structure between B7-1/B7-2 and CTLA-4 homodimers (Rudd et al. Immunol Rev. 229(1): 12-26 (2009); Sharpe et al. Nat Rev Immunol. 2(2):116-126, 2002). Activated CTLA-4 can bind to Phosphatidylinositol 3-Kinase (PI3K), the tyrosine phosphatases SHP1 and SHP2, and the serine/threonine phosphatase PP2A. Binding of CTLA-4 to PI3K generates positive signals in common with CD28. SHP1 and SHP2 dephosphorylate TCR signaling proteins, whereas PP2A targets phosphoserine/threonine residues and interferes with the activation of Akt. The association with the tyrosine phosphatases (e.g., SHP1, SHP2 and PP2A) can lead to the inhibitory actions of CTLA-4 (Podojil et al. Immunol Rev. 229(1): 337-355, 2009; Dustin and Depoil Nat Rev Immunol. 11(10): 672-684, 2011).

The CTLA-4 pathway is involved in modulating, e.g., inhibiting, various aspects of T cell responses. For example, CTLA-4 antagonizes B7-CD28-mediated costimulatory signals. Both CD28 and CTLA-4 bind to B7, but CTLA-4 has a much higher affinity for B7 than does CD28. Signaling through CTLA-4 inhibits IL-2 mRNA production and inhibits cell cycle progression. As another example, CTLA-4 inactivates T cells by delivering a negative signal. As yet another example, CTLA-4 interacts with TCR-CD3 complex at the immunological synapse or the proteins involved in downstream signaling after TCR activation.

The activation of T cells by antigen—MHC-II complex triggers a cascade of signaling events, e.g., phosphorylation of the Protein Tyrosine Kinases (PTKs) belonging to the Src and Zeta-Chain-Associated Protein Kinase (SYK ZAP70) families. The activation of T cells can also result in phosphorylation of the Immunoreceptor Tyrosine-based Activation Motifs (ITAMs) on the TCR-CD3 complex by Lck (attached to CD4 or CD8) and Fyn. Lck and Fyn are both members of the Src family of kinases. The phosphorylated ITAMs can bind the SH2 domains of ZAP70 SYK and subsequently phosphorylate and activate ZAP70 and SYK. This phosphorylation amplifies signals from the TCR through the activation of the adaptor proteins. These adaptor proteins, as part of the CTLA-4 pathway, include, e.g., Linker Activator for T-Cells (LAT), SH2 Domain-Containing Leukocyte Protein-76 (SLP76), Growth Factor Receptor-Bound Protein-2-Related Adaptor Protein-2 (GADS), T-Cell Receptor Interacting Molecule (TRIM), and enzymatic effectors, e.g., Phospholipase-C-Gamma1 (PLC-gamma 1). CTLA4 interacts with the ITAMs present on the TCR and CD3 and can disrupt the cascade of signals that lead to activation of the T-Cell. ZAP70, SYK and Fyn can be directly inhibited by the interaction of CTLA-4. CTLA4 can also bind to CD80/86 ligands.

The inhibitory effect by of CTLA-4 can be achieved by acting on downstream signaling as well. For example, CTLA-4 can bind to Clathrin adaptor complexes, e.g., Adaptor Protein-1 (AP1) and Adaptor Protein-2 (AP2), through its nonphosphorylated Tyr-Val-Lys-Met motif. AP1 and AP2 regulate the lysosomal degradation and endocytosis of CTLA4, respectively. CTLA4 can also function by physically disturbing the assembly or organization of molecules in the synapse, e.g., by sequestration of proteins involved in signal transduction away from the immunological synapse, thereby reducing the resultant signaling.

A CTLA-4 pathway can include any of the molecules, e.g., CTLA-4, or down-stream or up-stream of CTLA-4, e.g., as described above. In some embodiments, the CTLA-4 pathway include one or more of Zap70, PIK3R2, CD247, AKT2, NFATC2, LAT, LCK, FYN, CD3D, LAT, and TRAC.

### Cell Markers

Human hematopoietic cells can be characterized by various cell markers, as well as by gene expression analysis (Novershtern, et al. Cell 144(2):296-309, 2011, the contents of which are incorporated herein by reference). Cell markers for various hematopoietic cell populations include, but are not limited to, those provided below:

| | |
|---|---|
| CELL POPULATION | EXEMPLARY CELL MARKERS |

Hematopoietic Stem Cells

| | |
|---|---|
| HSC1 | lin−, CD133+, CD34dim |
| HSC2 | lin−, CD38−, CD34+ |

Ery Cells

| | |
|---|---|
| MEP | CD34+, CD38+, IL-3Rα−, CD45RA− |
| Ery1 | CD34+, CD71+, GlyA− |
| Ery2 | CD34−, CD71+, GlyA− |
| Ery3 | CD34−, CD71+, GlyA+ |
| Ery4 | CD34−, CD71^{lo}, GlyA+ |
| Ery5 | CD34−, CD71−, GlyA+ |

Mega Cells

| | |
|---|---|
| MEP | CD34+, CD38+, IL-3Rα−, CD45RA− |
| Mega1 | CD34+, CD41+, CD61+, CD45− |
| Mega2 | CD34−, CD41+, CD61+, CD45− |

Granulocyte/Monocyte Cells

| | |
|---|---|
| CMP | CD34+, CD38+, IL-3Rα^{lo}+, CD45RA− |
| GMP | CD34+, CD38+, IL-3Rα^{lo}+, CD45RA+ |
| Gran1 | CD34−, SSC^{hi}, CD45+, CD11b−, CD16− |
| Gran2 | CD34−, SSC^{hi}, CD45+, CD11b+, CD16− |
| Gran 3 | FSC^{hi}, SSC^{hi}, CD16+, CD11b+ |
| Mono1 | CD34−, CD33+, CD13+ |
| Mono2 | FSC^{hi}, SSC^{lo}, CD14+, CD45dim |
| Eos2 | FSC^{hi}, SSC^{lo}, IL3Rα+, CD33dim+ |
| Baso1 | FSC^{hi}, SSC^{lo}, CD22+, CD123+, CD33+/−, |
| | CD45dim |

DC

| | |
|---|---|
| Denda2 | HLA DR+, CD3−, CD14−, CD16−, CD19−, |
| | CD56−, CD123−, CD11c+ |
| Denda1 | HLA DR+, CD3−, CD14−, CD16−, CD19−, |
| | CD56−, CD123+, CD11c− |

B Cells

| | |
|---|---|
| Pre-BCell2 | CD34+, CD10+, CD19+ |
| Pre-BCell3 | CD34−, CD10+, CD19+ |
| BCella1 | CD19+, IgD+, CD27− |
| BCella2 | CD19+, IgD+, CD27+ |
| BCella3 | CD19+, IgD−, CD27− |
| BCella4 | CD19+, IgD−, CD27+ |

NK Cells

| | |
|---|---|
| NKa1 | CD56−, CD16+, CD3− |
| NKa2 | CD56+, CD16+, CD3− |
| NKa3 | CD56−, CD16−, CD3− |
| NKa4 | CD14−, CD19−, CD3+, CD1d+ |

T Cells

| | |
|---|---|
| TCell2 | CD8+, CD62L+, CD45RA+ |
| TCell1 | CD8+, CD62L−, CD45RA+ |
| TCell3 | CD8+, CD62L−, CD45RA− |
| TCell4 | CD8+, CD62L+, CD45RA− |
| TCell6 | CD4+, CD62L+, CD45RA+ |
| TCell7 | CD4+, CD62L−, CD45RA− |
| TCell8 | CD4+, CD62L+, CD45RA− |

The global transcriptional profiles of each group of hematopoietic cells were determined, and are consistent with the established topology of hematopoietic differentiation (Novershtern, et al. Cell 144(2):296-309, 2011). In some embodiments, a gene associated with or specific for a cell type described herein, e.g., B cells, T cells, erythrocytes (e.g., early erythrocytes or late erythrocytes), granulocyte/monocyte progenitors, and hematopoietic stem cells, is a gene encoding the cell marker described herein. In some embodiments, a gene associated with or specific for a cell type described herein, e.g., B cells, T cells, erythrocytes (e.g., early erythrocytes or late erythrocytes), granulocyte/monocyte progenitors, and hematopoietic stem cells, can be determined, e.g., based on the co-expression the gene to be determined and one or more cell markers described herein.

### Gene Expression Assays

The methods described herein can include one or more steps of evaluating the expression levels of one or more genes, e.g., one or more genes described herein, e.g., one or more genes associated with, or specific for, a cell type, e.g., B cells, T cells, erythrocytes (e.g., early erythrocytes or late erythrocytes), granulocyte/monocyte progenitors, and hematopoietic stem cells. In some embodiments, the level of mRNA is determined. In some embodiments, the level of protein is determined. The level of mRNA or protein can be compared to a standard, e.g., a standard described herein.

The level of mRNA corresponding to a gene, e.g., a gene described herein, in a cell, e.g., a cell described herein, e.g. a B cell, a T cell, an erythrocyte (e.g., an early erythrocyte or a late erythrocyte), a granulocyte/monocyte progenitor, and a hematopoietic stem cell, can be determined, e.g., by in vitro or in situ formats.

Nucleic acid probes for the genes described herein can be used in hybridization or amplification assays that include, but are not limited to, Northern analyses, polymerase chain reaction analyses and probe arrays. One method for the detection of mRNA levels involves contacting the mRNA with a nucleic acid molecule (probe) that can hybridize to the mRNA encoded by the gene being detected. The nucleic acid probe can be, for example, a full-length nucleic acid for the gene being detected or a portion thereof, such as an oligonucleotide of at least 7, 10, 15, 30, 50, 100, 250 or 500 nucleotides in length and sufficient to hybridize under stringent conditions to the mRNA, cDNA, or portions thereof. The probes can be labeled with a detectable reagent to facilitate identification of the probe. Useful reagents include, but are not limited to, radioactivity, fluorescent dyes or enzymes capable of catalyzing a detectable product.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by a gene described herein.

The level of mRNA in a sample that is encoded by a gene described herein can be evaluated with nucleic acid amplification, e.g., by RT-PCR (U.S. Pat. No. 4,683,202), ligase chain reaction (Barany, Proc. Natl. Acad. Sci. USA 88:189-193, 1991), self sustained sequence replication (Guatelli et al., Proc. Natl. Acad. Sci. USA 87:1874-1878, 1990), transcriptional amplification system (Kwoh et al., Proc. Natl. Acad. Sci. USA 86:1173-1177, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule including the nucleotide sequence flanked by the primers.

For in situ methods, a cell or tissue sample can be prepared/processed and immobilized on a support, typically a glass slide, and then contacted with a probe that can hybridize to mRNA that is encoded by the gene being analyzed.

A variety of methods can be used to determine the level of protein encoded by a gene, e.g., a gene described herein, in a cell, e.g., a cell described herein, e.g. a B cell, a T cell, an erythrocyte (e.g., an early erythrocyte or a late erythrocyte), a granulocyte/monocyte progenitor, and a hematopoietic stem cell. In general, these methods include contacting an agent that selectively binds to the protein, such as an antibody, with a sample to evaluate the level of protein in the sample. In one embodiment, the antibody includes a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of antibodies that can be used to detect a protein encoded by a gene described herein, e.g., a gene associated with or specific for B cells, T cells, erythrocytes (e.g., early erythrocytes or late erythrocytes), granulocyte/monocyte progenitors, and hematopoietic stem cells, are known in the art.

The detection methods for determining gene expression levels can also include methods which detect protein levels in a biological sample in vitro as well as in vivo. In vitro techniques for detection of protein include enzyme linked immunosorbent assays (ELISAs), immunoprecipitations, immunofluorescence, enzyme immunoassays (EIA), radioimmunoassays (RIA), and Western blot analysis. In vivo techniques for detection of proteins include introducing into a subject a labeled antibody against the protein. For example, the antibody can be labeled with a radioactive marker, e.g., a radioisotope) whose presence and location in a subject can be detected by standard imaging techniques. A radioisotope can be an α-, β-, or γ-emitter, or a β- and γ-emitter. Examples of radioisotopes that can be used include, but are not limited to: yttrium (⁹⁰Y), lutetium (¹⁷⁷Lu), actinium (²²⁵Ac), praseodymium, astatine (²¹³At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In) technetium (⁹⁹mTc), phosphorus (³²P), carbon (¹⁴C), and tritium (³H).

Correlative Functions Some of the methods, systems and databases described herein feature correlative functions. The following section provides additional details, specific embodiments and alternatives for correlative functions. These are not limiting but are rather exemplary. They can optionally be incorporated into methods, databases, or systems described herein.

### Correlative Functions

A correlative function can relate X to Y, where X is a value for an element related to gene expression and Y is a value for an element related to the clinical score and allows adjustment of the value for X to select or identify a value for Y or the adjustment of the value for Y to select or identify a value for X. By way of example, X can be a value of gene expression level, a value of gene copy number, or a value of cell type, and in one or more of those cases, Y can be a clinical score associated with disease severity, disease progression, clinical outcome, or prognosis.

### Exemplary Computer Implementation

The methods and articles (e.g., systems or databases) described herein need not be implemented in a computer or electronic form. A database described herein, for example, can be implemented as printed matter.

Where appropriate, the systems and the functional operations described in this specification can be implemented in digital electronic circuitry, or in computer software, firmware, or hardware, including the structural means disclosed in this specification and structural equivalents thereof, or in combinations of them. The techniques can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple computers. A computer program (also known as a program, software, software application, or code) can be written in any form of programming language, including compiled or interpreted languages, and it can be deployed in any form, including as a standalone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification can be performed by one or more programmable processors executing one or more computer programs to perform the described functions by operating on input data and generating output. The processes and logic flows can also be performed by, and apparatus can be implemented as special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, the processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, aspects of the described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The techniques can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network.

The relationship of client and server arises by virtue of computer programs running on the respective computers and having a client-server relationship to each other.

A number of implementations have been described. Nevertheless, it will be understood that various modifications can be made without departing from the spirit and scope of the described implementations. For example, the actions recited in the claims can be performed in a different order and still achieve desirable results. Accordingly, other implementations are within the scope of the following claims.

The invention is further illustrated by the following examples, which should not be construed as further limiting.

### Examples

### Example 1: Patient Stratification in Secondary-Progressive Multiple Sclerosis (SPMS) Using a Mixture of Experts Model

Multiple sclerosis (MS), particularly of the relapsing-remitting form (RRMS) has been extensively studied in the past years and various treatments have been developed. As of now, however, no therapy is effective against the more advanced stage of the disease, known as secondary-progressive MS (SPMS). One of the major reasons for this is an increased heterogeneity in SPMS patients. In this example, the heterogeneity of SPMS represented by whole blood molecular profiles and clinical disease severity scores was examined.

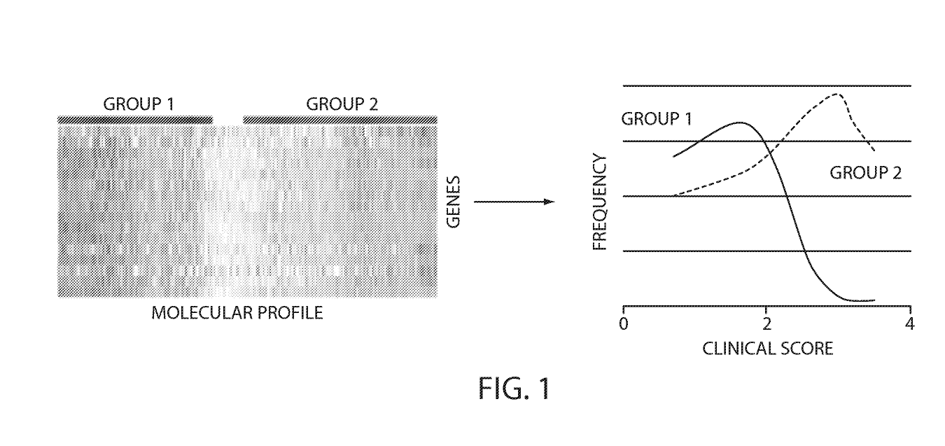

Traditionally, molecular profiles of patient cohorts are first analyzed independently of the disease score to identify molecularly uniform classes. Once distinct classes are identified, one checks for association with disease score or progression. This approach assumes that molecular subgroups directly reflect differences in disease severity and/or progression classes. An exemplary traditional paradigm of patient stratification is depicted in FIG. 1. In a recent paper Ottoboni et al. (Sci. Trans. Med., 4(153), 2012) applied this approach to blood profiles in a RRMS cohort and identified two molecular subclasses that correlate with time to relapse in RRMS patients treated with glatiramer acetate or IFNβ.

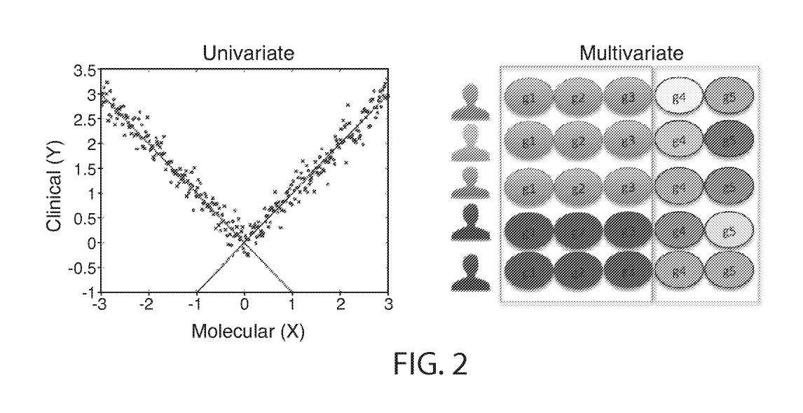

In this example, a new method for discovery of patient subclasses looking at the joint space of molecular markers and disease scores is described. The premise of this method is the supposition that in the joint space of molecular and disease scores, there may exist distinct subgroups of patients such that each group is characterized by a different dependence between molecular and disease scores. This concept is illustrated in a toy model in FIG. 2 (left) where a hypothetical relationship between the molecular variable X and clinical variable Y is depicted. Investigating just the space X of molecular markers or just the space Y of clinical variables does not reveal any subgroups. The underlying structure is only apparent in the joint X, Y space. When X and Y are multi-dimensional, as in the real world, de-convoluting the structure by looking at them individually becomes even harder.

In a multivariate setting (FIG. 2 (right)), there might be genes (g1, g2, g3) that are differentially expressed in the sub groups that act as the cleanest markers. However, different genes might be correlated with the disease score (or clinical outcome). The relationship between these genes and the clinical outcome may be different for the different subgroups, thereby indicating a difference in the underlying biology. This cannot be captured by traditional patient stratification methods that will tend to ignore g4 and g5 completely. Such complex relationships can be revealed by looking at the joint space of molecular and clinical variables.

The method described in this example looks at the joint distribution of patients in X (molecular) and Y (clinical) space, employs a mixture of linear models to explain the dependence between Y and X, and identifies optimal number of patient subgroups. Formally, given the number of clusters ‘K’, this is represented as the likelihood maximization problem:

$$Θ = {arg max}_{Θ} P ( Y , X )$$

$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$

where Θ represents the set of parameters we use to describe the joint distribution. This includes parameters for the linear regression used to describe P(Y|X,cₘ), parameters for describing the clusters P(X|cₘ) and P(cₘ). To prevent overfitting, a regularized Expectation-Maximization (EM) algorithm was used to learn a sparse set of parameters and select the optimal number of clusters via the Bayesian Information Criterion (BIC).

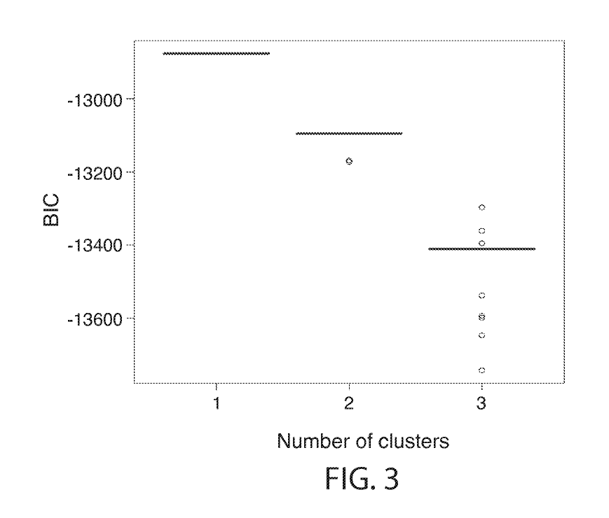

This approach was applied to the blood profiles of 190 SPMS patients characterized either by the Expanded Disability Status Scale (EDSS) or Multiple Sclerosis Severity Score (MSSS). Whole blood was collected in PAXGene tubes and profiled on Affymetrix platform hghgu133plusPM. Dimensionality of the molecular profiles was reduced by using non-negative matrix factorization (NMF). Number of reduced dimensions (factors) was chosen by maximizing cophenetic correlation. Twenty five factors are optimal in NMF. Two clusters are optimal—there were signs of overfitting at 3 clusters, as can be seen in FIG. 3. Clusters obtained were stable, as assessed through multiple runs of the algorithm.

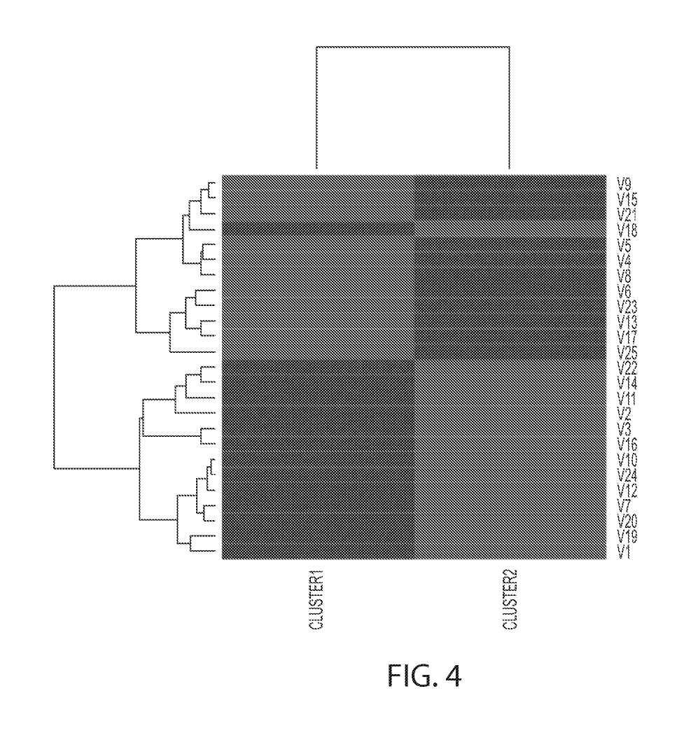

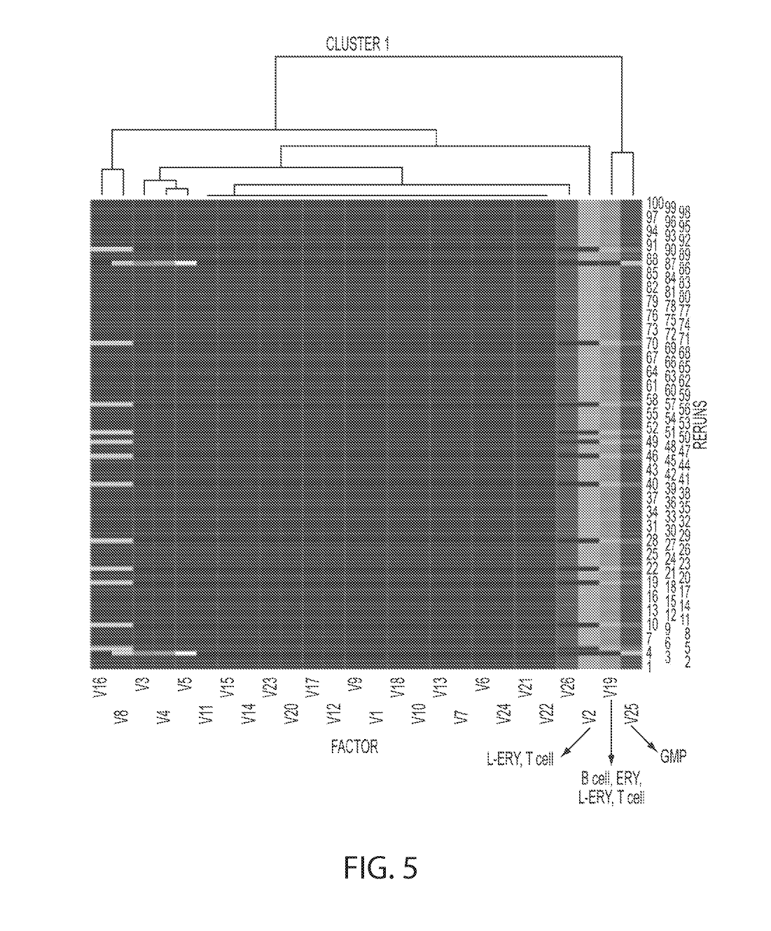

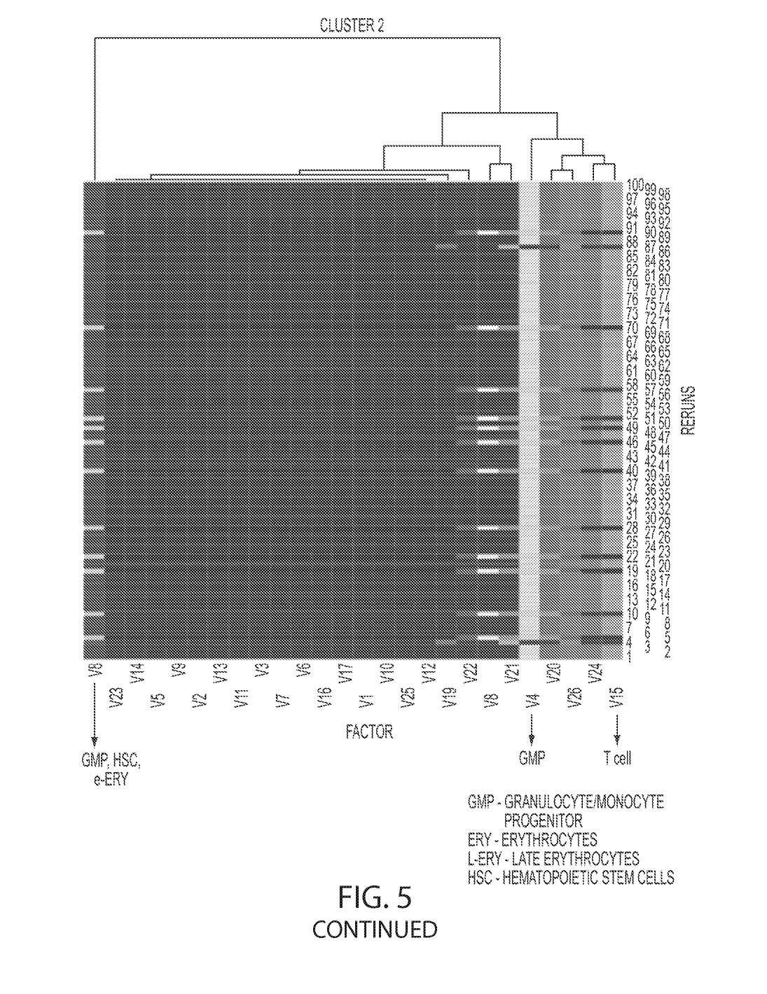

An exemplary map of the factors to different cell types using cell-specific expression pattern from D-MAP data is shown in FIG. 4. Exemplary different dependence between molecular factors and disease severity in the two sub-groups is shown in FIG. 5.

Two subgroups of patients were distinctly identified by high or low expression of B cells, T cells, GMP cells and early-Erythrocyte (e-ERY) cells. Within each subgroup a different molecular signature best reflects the disease score. While in one subgroup B cells, GMP and ERY cells are correlated with disease severity (MSSS), in the other, GMP and ERY cells are correlated. It is important to note that relationships between the cell types and disease severity are different in the two subgroups. Also, since different features (or factors in the Non-negative matrix factorization) correlate with MSSS, different probes that are specific to the factors (but specific to similar cell types) correlate with MSSS. So, even though GMP is correlated in both sub groups, the actual probes that are correlated are different in both cases. Furthermore, lower BIC for the model indicates that the best model is better than the traditional approach that associates each signature with disease score in the entire cohort.

This example demonstrates, among other things, that multivariate biomarkers provide a new and useful approach for stratifying heterogeneous populations, e.g., MS patient populations. Different patient's sub-groups may be characterized by different dependence between molecular and clinical outcomes; thereby indicating different underlying disease biology. This can only be discovered by analyzing the joint space of molecular and clinical profiles. Traditional paradigm of patient stratification fails to capture this. Furthermore, in a multivariate setting, different features might be responsible for clustering and prediction of clinical outcome.

### REFERENCES

Gershenfeld, N., Schoner, B. and Metois, E., (1999) Cluster-weighted modelling for time-series analysis, Nature, 397: 329-332.
Jakkola, T., Machine Learning lecture notes, MIT.
Novershtern, N., Subramanian, A., Lawton, L., et al, (2011) Densely interconnected transcriptional circuits control cell states in human hematopoiesis, Cell, 144: 296-309.

### Example 2: Patient Stratification in Multiple Sclerosis (MS)

### Summary

The objective of this Example is to identify subgroups of MS patients with more severe disease and distinct phenotypes. In particular, this Example aims to identify molecular characteristics of secondary-progressive (SPMS) patients.

Towards this goal, the heterogeneity of MS represented by whole blood molecular profiles and clinical disease severity scores was examined. A new method for discovery of patient subclasses by looking at the joint space of molecular markers and disease scores was used. The premise of the method is that in this joint space, there may exist distinct subgroups of patients characterized by different dependencies between molecular and disease scores. The distribution of patients in the joint molecular (X) and clinical (Y) space was examined, a mixture of linear models was employed to explain the relationship between Y and X, and the optimal number of patient subgroups was identified. For molecular markers, genes differentially expressed in SPMS vs. healthy samples were considered.

This approach was applied to blood profiles of 190 SPMS patients characterized by Multiple Sclerosis Severity Score (MSSS). Two subgroups were identified within the cohort. Within each subgroup a different molecular signature best reflects the clinical score.

### Data Collection

The data used for this analysis are as follows: SPMS population (Accelerated Cure Project (ACP) group; n=106; molecular data: yes (whole blood); clinical data: yes; MRI: no; longitudinal: no; matched controls: yes (n=29); under treatment: yes).

### ProbeSelect

Due to the heterogeneity present in SPMS, naïve differential expression approaches do not yield any differentially expressed genes. A new algorithm, ProbeSelect, was developed to identify differentially expressed genes in heterogeneous populations. 1754 probes were selected based on the differential expression in SPMS-vs-control using this method.

The ProbeSelect algorithm computes a z-score for each probe for patient with respect to the means calculated from the healthy controls. For each probe, the algorithm counts the number of patients with absolute z-scores that are greater than the cutoff (e.g., 1.5). P value is used to quantify how likely these numbers of patients can be selected above the cutoff by chance. Probes that are statistically significant are selected after the P value is corrected for multiple hypothesis testing. ProbeSelect is described, e.g., in Hosur et al. Bioinformatics 30(4): 574-575, 2014.

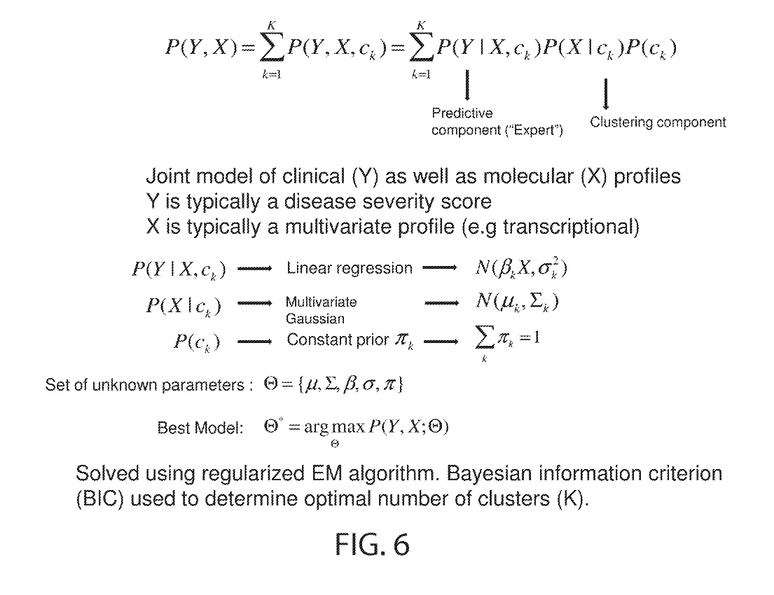

A mixture of experts approach was used to model joint distribution of molecular and clinical data. Differentially expressed genes from SPMS vs. healthy subjects were first reduced to a lower dimensional space using Non-negative Matrix Factorization (NMF). Each “factor” in the reduced dimensional space is a linear combination of the original differentially expressed genes. Sub-groups are characterized by different dependence between clinical and molecular variables. Different features can be important for stratification and clinical outcome. A brief illustration of the mixture of experts model for patient stratification is shown in FIG. 6.

### SPMS Subgroups

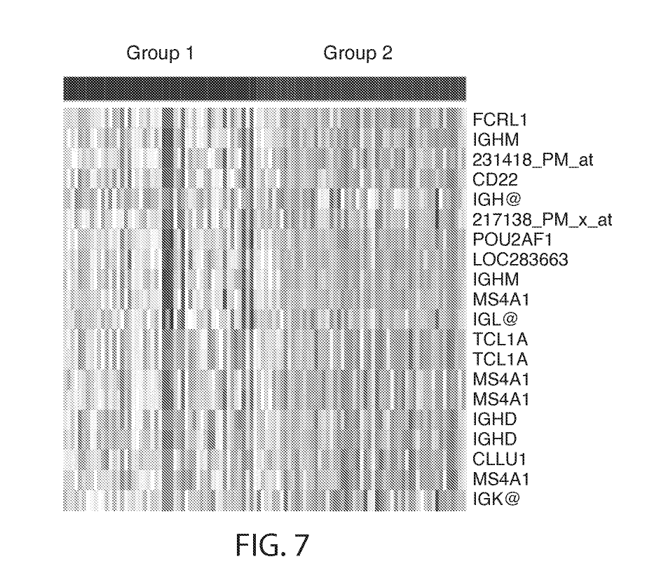

MSSS was used as the clinical variable. Two subgroups were found in the cohort: 51 and 55 patients in each subgroup. FIG. 7 shows the top 20 differentially expressed probes between ACP (SPMS) subgroups. The differentially expressed genes or loci include, e.g., FCRL1, IGHM, 231418_PM_at, CD22, IGH@, 217138_PM_x_at, POU2AF1, LOC283663, IGHM, MS4A1, IGL@, TCL1A, MS4A1, IGHD, CLLU1, and IGK@.

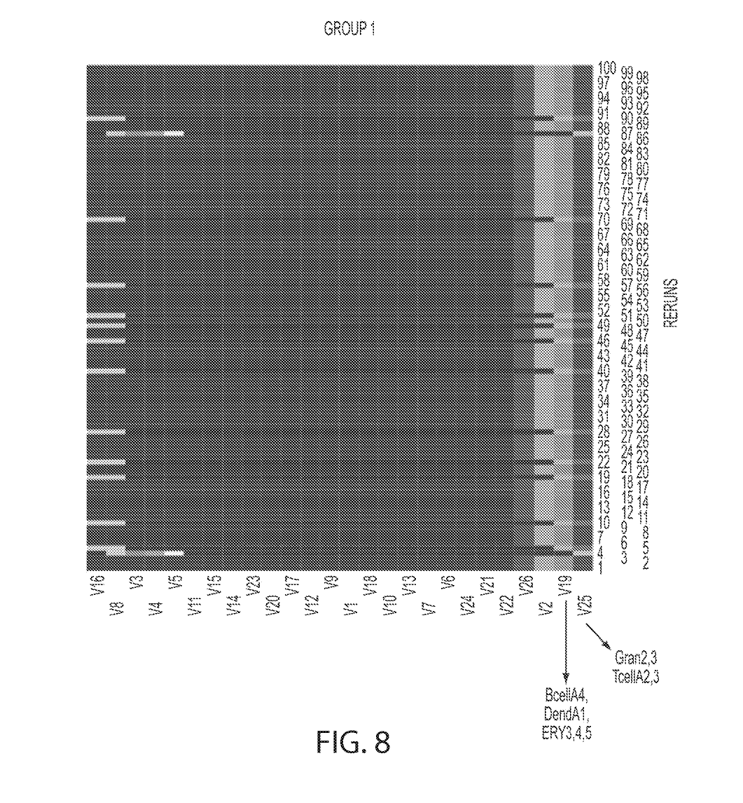

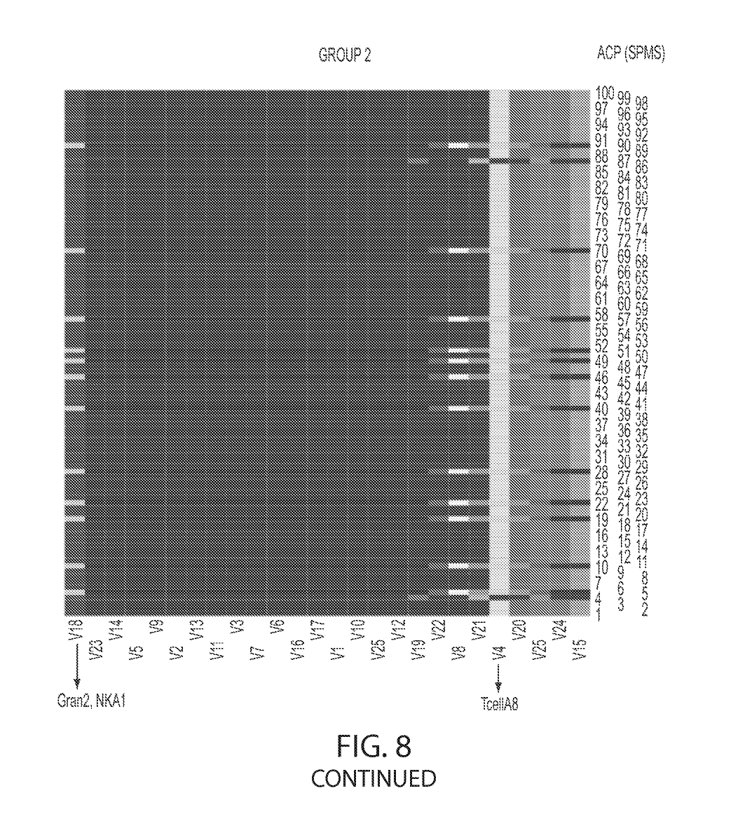

As shown in FIG. 8, there is different dependence between molecular factors and disease severity in the subgroups. The factors (or probes) were mapped to different cell types as described above. For example, B cells (type A4), dendritic cells (type A1), erythrocytes (types 3, 4 and 5), and T cells (types A2 and A3) were identified in cluster 1; and granulocytes, NK cells (type A1), and T cells (type A8) were indicated in cluster 2.

### Conclusions

Multivariate biomarkers provide a new approach to stratify heterogeneous populations. Different subgroups were characterized by different dependence between molecular and clinical outcomes; thereby indicating different underlying biology. This can only be discovered by analyzing the joint space of molecular and clinical profiles. Traditional paradigm of patient stratification fails to capture this. In a multivariate setting, different features might be responsible for clustering and predictive of clinical outcome.

### Example 3: ExpertMIX Stratification of Molecular Profiles and Clinical Disease Variability in RRMS and SPMS Patients

### Summary

The disease course and severity of relapsing multiple sclerosis (MS) patients is typically heterogeneous. At disease onset, patients have relapsing-remitting MS (RRMS), characterized by periodic relapses of neurological disability, from which they may or may not fully recover. Over years to decades, about 85% patients develop secondary-progressive MS (SPMS), characterized by insidious neurological worsening independent of relapses, though some experience a milder form of RRMS over their life time and never enter a progressive phase.

In this Example, whole blood transcriptional profiles of RRMS and SPMS patients were used to study differences between these two populations. A method was developed to investigate patient heterogeneity in the joint space of molecular profiles and clinically defined MS Severity Score (MSSS). In both the RRMS and SPMS cohorts, two subgroups, A and B, were identified. RRMS_{A} and SPMS_{A} are characterized by a more severe disease and higher expression of transcripts from similar lymphocytic pathways than RRMS_{B} and SPMS_{B}, respectively. For example, RRMS_{A} subgroup tends to have a shorter time to disability progression and a higher annualized relapse rate (ARR).

One lymphocytic pathway exception is CTLA4, which is differentially expressed between RRMS_{A} and RRMS_{B}, but not between SPMS_{A} and SPMS_{B}. In RRMS_{A} but not RRMS_{B}, T-cell and granulocyte expression pathways correlate with MSSS. In SPMS_{A} both B-cell and dendritic cell expression pathways correlate with MSSS, while there is no correlation with any molecular signatures for SPMS_{B}. Together, these findings support distinct subpopulations of MS characterized by different clinical and molecular profiles. Two molecular/clinical subgroups of MS can be defined in both RRMS and SPMS, characterized by differential expression of lymphocyte activation pathways, and the CTLA4 signature identifies RRMS patients at higher risk of short term disease activity.

### Results

Subgroups of RRMS and SPMS patients were identified by the expertMIX stratification method, which classifies patients in the joint molecular and clinical space, based on whole blood molecular profiles. The MS Severity Score (MSSS), which takes into account disability level and time since disease onset, represents a spectrum of clinical disease severity (Roxburgh, et al. 2005 Neurology 64(7):1144-51). Using an independent computational approach, this study identifies two molecular subclasses of RRMS. As shown in Table 3, at baseline the two classes do not differ in demographics or disease severity. It was found that these two subgroups differ in their expression of lymphocyte activation pathways. Moreover, the whole blood molecular profiles of SPMS patients revealed that two groups of SPMS patients are also defined by differences in very similar lymphocyte activation pathways, independent of disease-modifying treatment.

TABLE 3
Characteristics of RRMS_{A} and RRMS_{B} patient subgroups
from the combined placebo arms of the DEFINE and
CONFIRM studies.

| | RRMS_{A} | | RRMS_{B} | | |
|---|---|---|---|---|---|
| | (n = 269) | | (n = 281) | | |
| | mean | sd | mean | sd | **p.value |
| Female N (%) | 191 (71) | | 210 (74) | | 0.371 |
| Age (years) | 37.92 | 8.83 | 38.55 | 9.44 | 0.632 |
| Symptom-duration at | 8.20 | 5.88 | 8.59 | 6.92 | 0.632 |
| sampling (years) | | | | | |
| EDSS | 2.63 | 1.18 | 2.53 | 1.22 | 0.192 |
| MSSS | 4.04 | 2.13 | 3.88 | 2.28 | 0.536 |
| Months since last | 6.28 | 5.74 | 6.79 | 7.56 | 0.400 |
| relapse | | | | | |
| Relapses in prior year* | 1.26 | 0.57 | 1.37 | 0.75 | 0.243 |
| Study (DEFINE) | 134 (50) | | 138 (49) | | |
| N (%) | | | | | |

*The number of relapses in prior year were assessed using a negative binomial model and linear model for other variables.
**p-value is adjusted for time of symptom duration, age and gender (unless outcome is variable).

### RRMS and SPMS Subgroups Defined Jointly by Molecular Profiles and Disease Severity

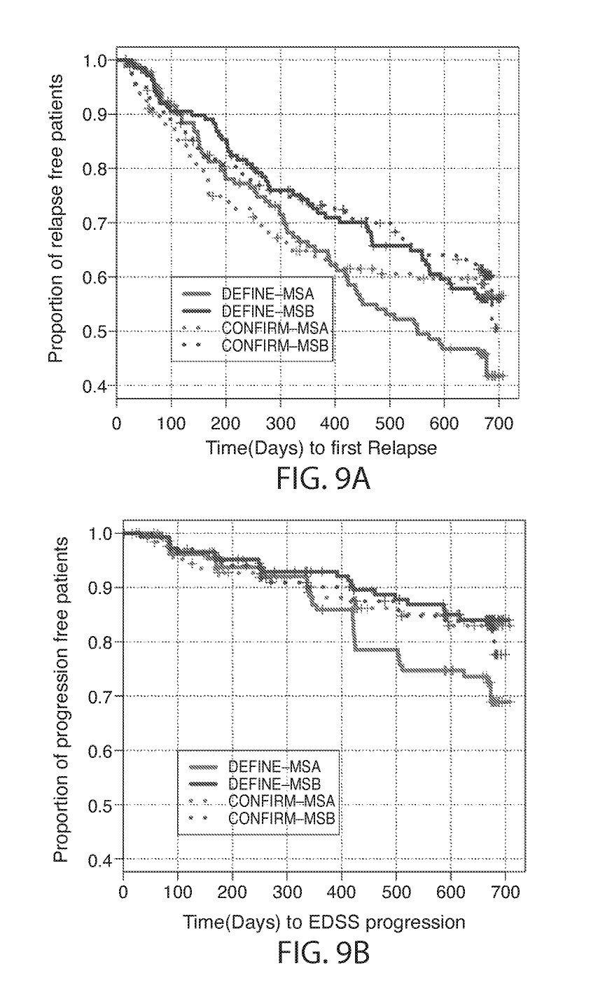

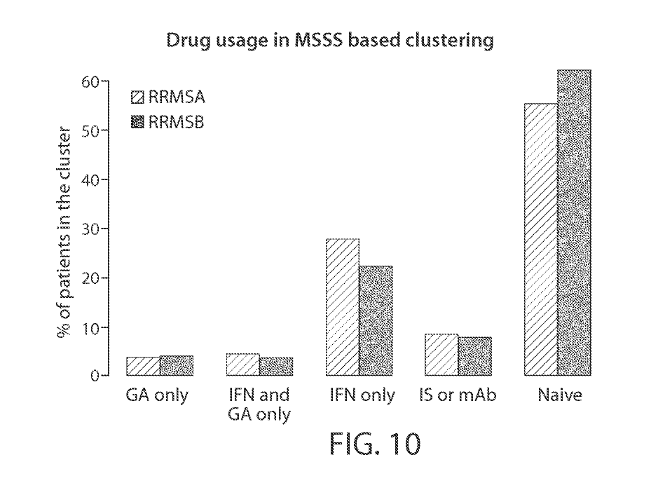

Application of the expertMIX stratification method to the RRMS cohort (N=550) revealed two distinct subgroups. Table 3 summarizes demographic and clinical characteristics of RRMS subjects belonging to the two molecular subgroups at baseline. At baseline there are no significant differences between the subgroups. ARR corrected for Expanded Disability Status Scale (EDSS), region, age and number of relapses in prior year is 0.48 95% CI=[0.22, 0.89] and 0.41 95% CI=[0.21,0.78] for the RRMS_{A} and RRMS_{B} subgroups, respectively (p=0.26). The RRMS_{A} tends to have a shorter time to disability progression, as defined by 12-week confirmed worsening of EDSS, p=0.06, corrected for age, region, baseline EDSS and relapses in the prior year and stratified by study (DEFINE and CONFIRM). As shown in FIGS. 9A-9B, this difference is significant (p=0.01) in the DEFINE study population, which was characterized by relatively greater on-study disease activity (Fox, et al. 2012 N Engl J Med 367(12):1087-97; Gold, et al. 2012 N Engl J Med 367(12):1098-107). Time to first relapse also tends to be shorter in the RRMS_{A} (p=0.13). The association of the RRMS_{A} and RRMS_{B} subgroups with prior disease-modifying therapy use was not found (see FIG. 10).

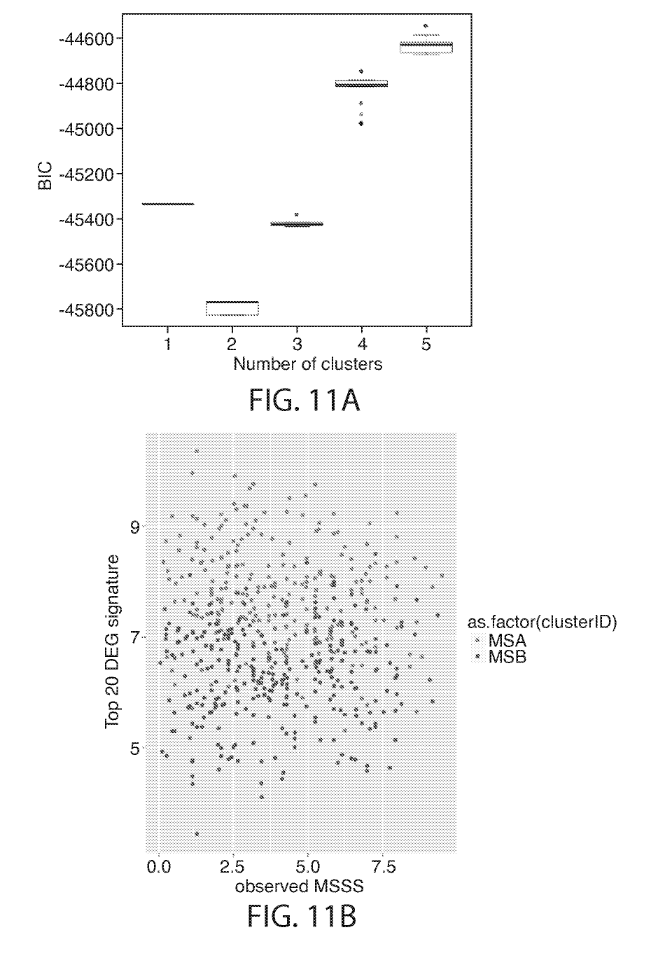

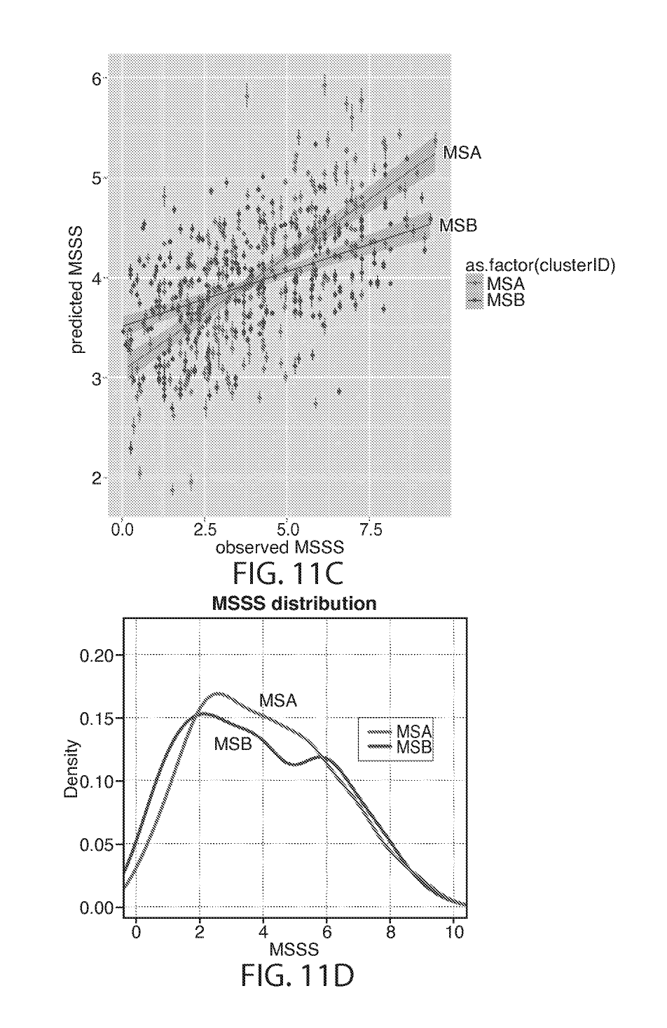

A mixture-of-experts representation of RRMS is shown in FIGS. 11A-11D. To select the optimal number of subgroups present in the population, a model selection procedure based on Bayesian Information Criteria (BIC) was adopted. Among the 1 to 5 subgroups (corresponding to 1-5 expert models) tested, the 2-subgroup model has the lowest—BIC—and clearly better fits the data than 1 or 3 subgroups (see FIG. 11A) ABIC>>10 is considered a highly significant difference (Kass and Raftery (1995) Journal of the American Statistical Association 90(430):773-795). Thus, 2-subgroups is an optimal model of molecular and clinical heterogeneity in the RRMS cohort. FIG. 11B shows samples in the molecular space defined by the top differentially expressed genes, between the two clusters. The differentially expressed genes separate the two subgroups even though they exhibit similar ranges for MSSS. FIG. 11C shows the relationship between MSSS modeled by two molecular characteristics and the observed MSSS. The molecular components that correlate with MSSS (the non-zero coefficients in (β_{RRMSA}, β_{RRMSB}) have very different relationships to MSSS in the two subgroups, with an angle between β_{RRMSA} and β_{RRMSB} of 108 degrees. An angle larger than 90 degrees indicates that the molecular components β_{RRMSA} and β_{RRMSA} have an opposite relationship with MSSS in the two subgroups. In RRMS_{A} the β_{RRMSA} molecular component is positively correlated with MSSS, while in cluster RRMS_{B} this correlation tends to be negative. The opposite holds true for the β_{RRMSB} molecular component, where in cluster RRMS_{B} it is positively correlated, with MSSS while in cluster RRMS_{A} the correlation tends to be negative. FIG. 11D shows that there is no significant difference in the distributions of MSSS between the two subgroups, although RRMS_{A} appears slightly shifted toward a higher MSSS range as compared to RRMS_{B}.

In addition to recognizing molecular subgroups, expertMIX also identifies the molecular factors that may explain clinical heterogeneity in these subgroups. Factors significantly associated with clinical heterogeneity are determined as described in the Methods. GeneSet enrichment-like (GSE) approach and D-MAP (differentiation map of hematopoiesis) (Novershtern, et al. (2011) Cell 144(2):296-309) data (described in the Methods) provide an interpretation of molecular factors associating with MSSS in terms of major hematopoietic cell types. In the RRMS_{A} sub-group, T-cells (A1 and A3) CD4+CD62L-CD45RA+ and CD4+CD62L-CD45RA−, and granulocytes 2, 3 (Myelocytes and Neutrophils) account for MSSS variability. In the RRMS_{B} subgroup much smaller fraction of MSSS variability may be explained by molecular factors, as represented by much smaller (2-3 orders of magnitude) linear coefficients.

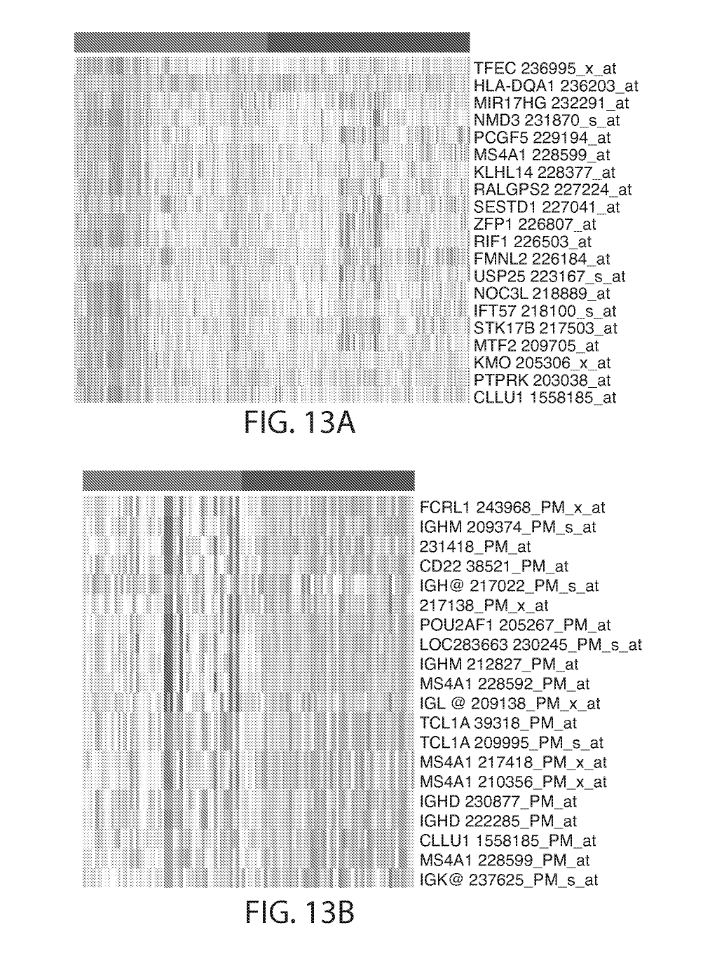

The top 20 transcripts that differentiate the subgroups of RRMS_{A} vs RRMS_{B} samples are shown in FIG. 13A.

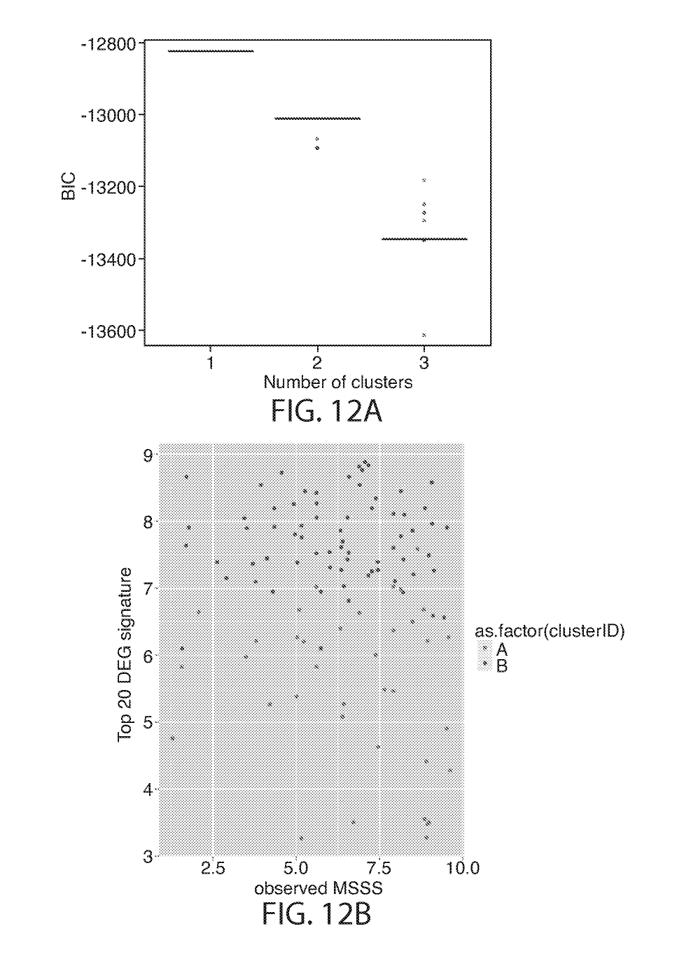

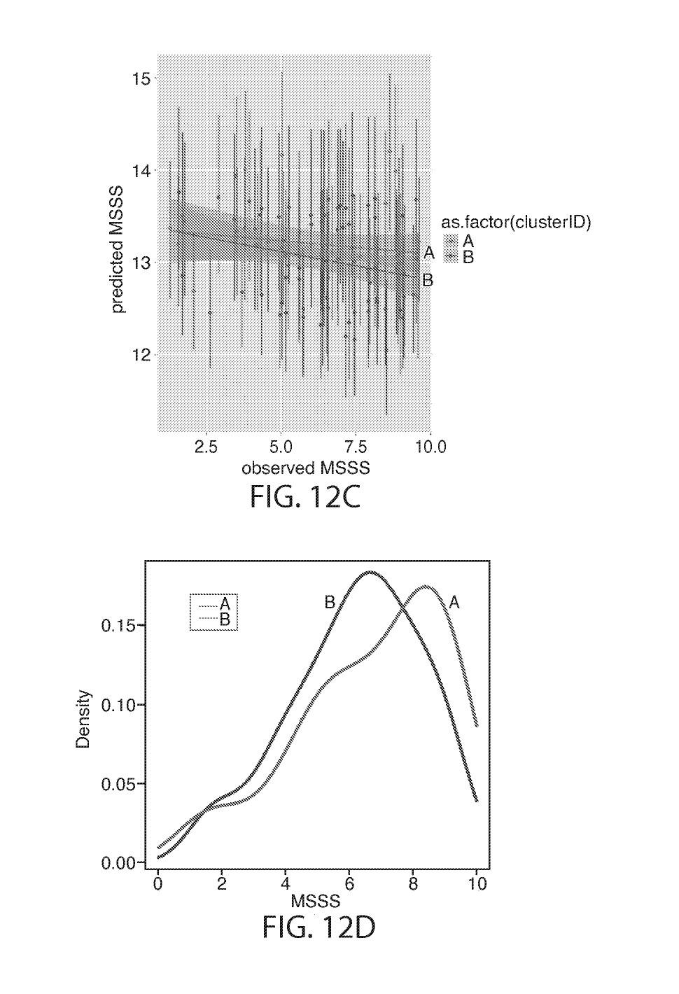

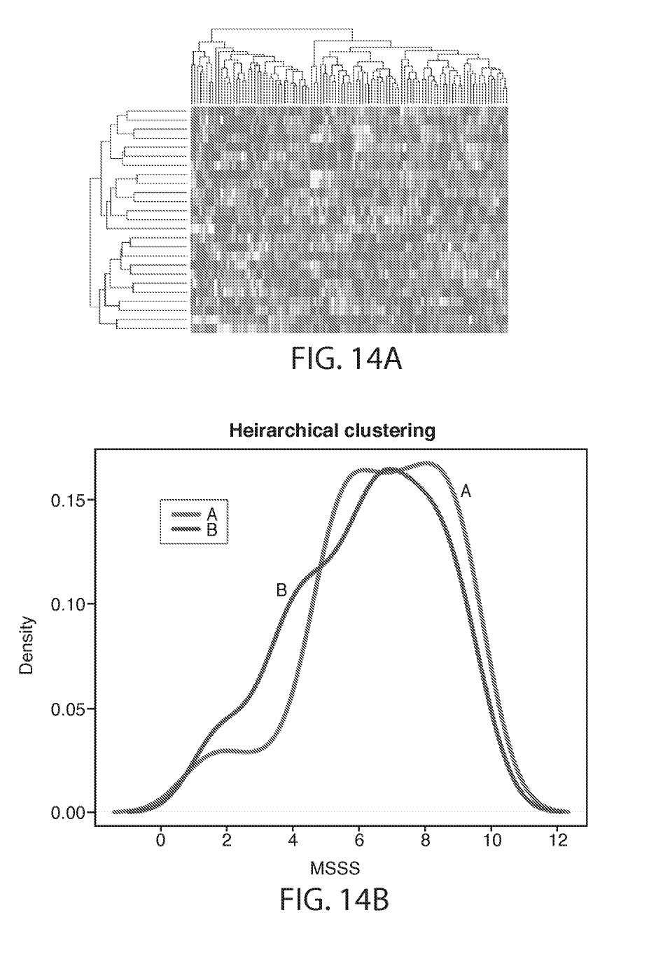

Applying the expertMIX stratification to the SPMS cohort (N=106), 2 subgroups differentiated by their molecular/clinical characteristics were discovered (see Table 4). BIC for 1-3 subgroups of SPMS samples is shown in FIGS. 12A-12D. FIG. 12A shows the BIC distributions for 1 to 3 mixture-of-experts models of molecular and clinical variability. Only BIC for up to 3 clusters was reported since higher cluster number resulted in very small and variable clusters of ˜10 patients. The BIC indicates that three molecular subgroups might better explain the MSSS variability in SPMS cohort. However, due to small sample size, BIC variability is large for 3 subgroups and therefore the more stable 2-subgroup model was chosen. BIC is significantly lower (ABIC>10 (Kass and Raftery (1995) Journal of the American Statistical Association 90(430):773-795)) for the 2 subgroup than one group indicating patients molecular heterogeneity. The top 20 transcripts differentiating cluster SPMS_{A} from SPMS_{B} are shown in FIG. 13B. These transcripts represent a projection in the molecular space that differentiated the subgroups. FIG. 12B shows SPMS samples projected into these axes. FIG. 12C shows large variability of modeled vs. observed MSSS. This may be due to several factors: more heterogeneous population, ongoing treatments, relatively small sample size and/or variability of clinical assessment in the context of an observational cohort. The distribution of MSSS is slightly shifted toward a higher MSSS in SPMS_{A} (p=0.19, FIG. 12D). Two molecular subgroups identified with conventional hierarchical clustering show no difference in the MSSS distributions (FIGS. 14A-14B).

TABLE 4
Characteristics of SPMS_{A} and SPMS_{B} patient subgroups

| | |
|---|---|
| SPMS_{A} | SPMS_{B} |
| (n = 37) | (n = 69) |

| | mean | sd | mean | sd | p.value |
|---|---|---|---|---|---|
| Female nN | 20(65) | | 49(71) | | 0.61 |
| (%) | | | | | |
| AGEAge | 54.4 | 10.4 | 55.4 | 7.8 | 0.55 |
| (years) | | | | | |
| Duration of | 14.4 | 11.9 | 14.0 | 8.9 | 0.55 |
| symptoms | | | | | |
| (years) | | | | | |
| EDSS | 5.9 | 1.6 | 5.4 | 1.7 | 0.26 |
| MSSS | 6.7 | 2.3 | 6.2 | 2.0 | 0.19 |
| Age at | 39.2 | 9.3 | 40.5 | 11.2 | 0.91 |
| diagnosis | | | | | |

*p.value is adjusted for symptom duration, age and gender (unless outcome variable). Data for prior relapses was not available for this cohort.

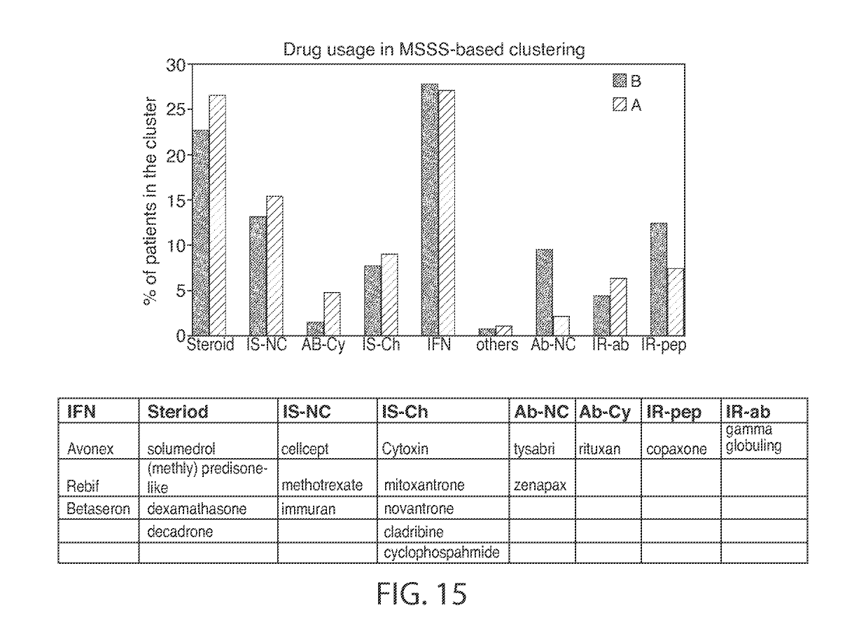

In both SPMS subgroups only nominal fraction of MSSS variability is explained by molecular factors, quantitated by linear coefficients very close to zero and 4 to 6 orders of magnitude smaller than in RRMS (FIG. 12D). Nevertheless several molecular factors significantly correlate with MSSS. In SPMS_{A} subgroup MSSS correlates with expression of transcripts representing granulocytes 2, 3 (Myelocytes, Neutrophils), Tcell-A1 (CD8+CD62L-CD45RA+) and erythrocytes (2, 3, 4, 5) lineages (Novershtern, et al. (2011) Cell 144(2):296-309). In SPMS_{B}, Tcell A1 and A7 (CD8+CD62L-CD45RA+ and CD4+CD62L-CD45RA−) erythrocytes (2, 3, 4, 5), lineages and granulocytes 2, 3 (Myelocytes, Neutrophils). Given that SPMS patients may be treated with one or more DMTs, the association of SPMS_{A} and SPMS_{B} subgroups with prior disease-modifying therapy use was investigated, but such association was not found (see FIG. 15). This finding is consistent with the observations from the RRMS cohort (FIG. 10).

### Differentially Expression of Pathways

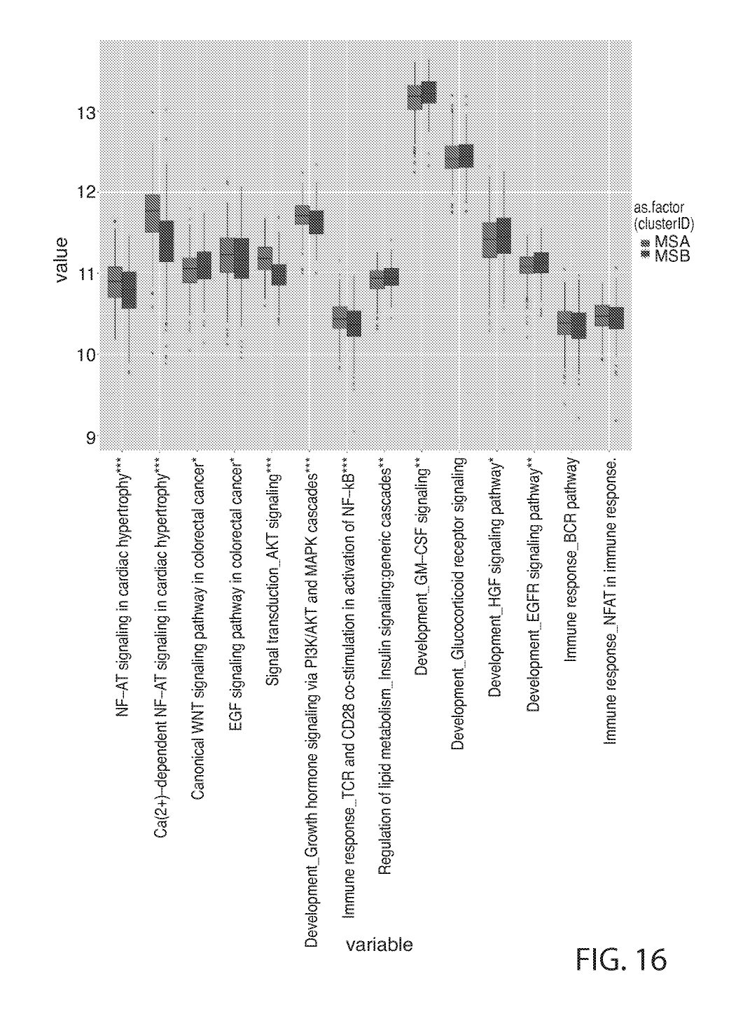

The list of genes differentially expressed between subgroups was identified as described in Methods. FIG. 16 shows pathway-level differential expression between RRMS_{A} and RRMS_{B} with most pathways significantly up-regulated in RRMS_{A}.

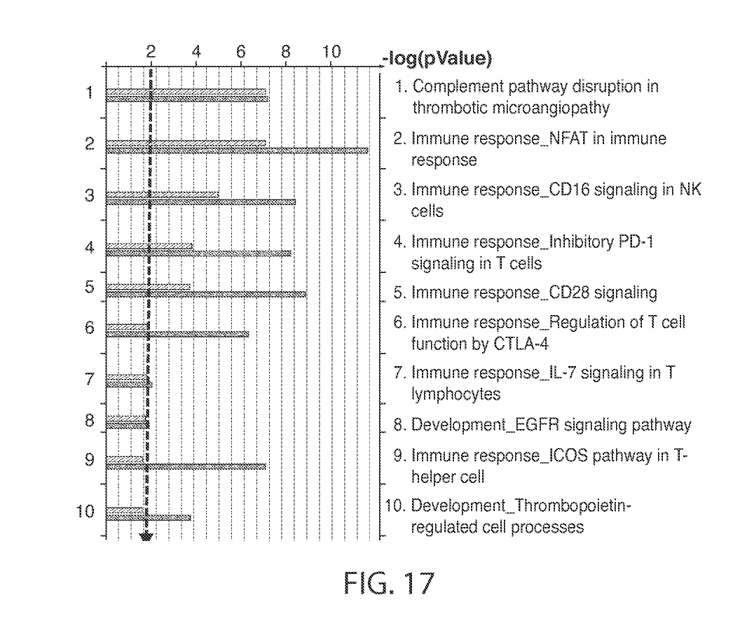

Differentially expressed pathways between two SPMS subgroups were also identified. Pathways enriched in the SPMS_{A} subgroup are very similar to those in RRMS_{A}. One notable difference is the CTLA4 pathway, which is differentially expressed in RRMS subgroups but not in SPMS, shown in FIG. 17. This observation indicates that in SPMS expression of the CTLA4 pathway is more uniform as compared to RRMS. The expression of the CTLA4 pathway signature was investigated in more detail. For example, the expression of 11 genes: -Zap70, PIK3R2, CD247, AKT2, NFATC2, LAT, LCK, FYN, CD3D, LAT, and TRAC, was assessed.

### Baseline CTLA4-Pathway Signature as a Marker of Relapse Activity in RRMS

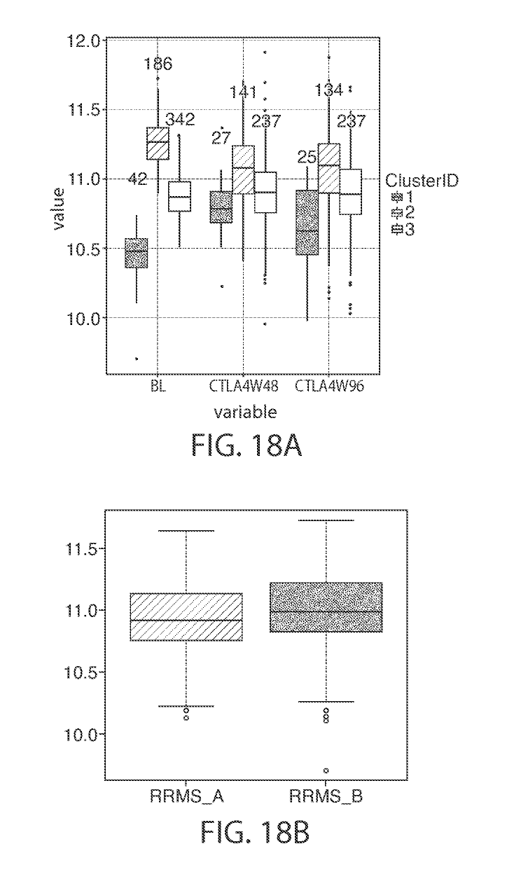

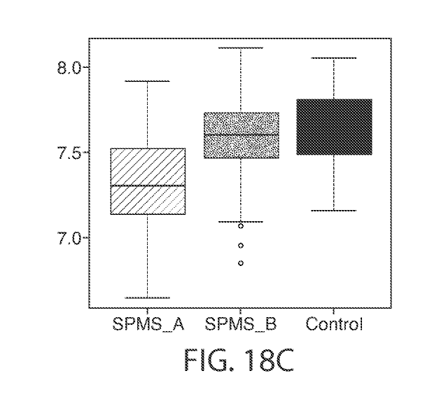

Given the uniformity of CTLA4 pathway expression in SPMS, the association of the CLTA4 pathway with disease activity in RRMS was explored by assessing relapse activity over the subsequent 2 years, as measured by ARR. ARR was modeled by the negative binomial distribution. With unsupervised hierarchical clustering of CTLA4-pathway expression at baseline, patients were assigned to three possible classes: CTLA4-MS₁, CTLA4-MS₂ or CTLA4-MS₃, with low, medium and high gene expression (FIGS. 18A-18C). After correction for baseline EDSS, age, study and the number of relapses in the prior year and region, a higher expression of CTLA4 signature is significantly associated with higher ARR: CTLA4 MS₁/MS₂/MS₃ ARR=0.109 CI(0.052-0.229)/0.341 CI(0.283-0.410)/0.373 CI(0.297-0.469). ARR is significantly lower in cluster MS₁ versus clusters MS₂ or MS₃, with p values of 0.0015 and 0.003, respectively. Using data collected in treatment naïve patients after 1 and 2 years, stability of the CTLA4 signature (FIGS. 18A-18C) was assessed in the majority of patients assessed at baseline. This observation suggests that the CTLA4 pathway is a better predictor of short-term disease activity, as reflected by ARR, than the RRMS_{A} vs. RRMS_{B} classification, in which RRMS_{A} tends to have higher ARR (p.value=0.26) and shorter time to confirmed disability progression.

Thus, this study has identified two molecularly distinct subgroups in RRMS. Additionally, this approach has focused the differences on more specific signatures (e.g., CTLA4-pathway) associated with short-term disease activity as measured by ARR. Given the small number of transcripts in the CTLA4-pathway signature, this approach can be more amenable to translation into clinical practice than genome-scale profiling. Further, the expertMIX stratification revealed an association of other immune cell (e.g., granulocytes, T-cell and dendritic cell) pathways with disease severity. These additional molecular associations suggest that other molecular/clinical variables can further define MS subgroups.

In this cross-sectional approach MSSS provides information on the disease severity spectrum, as it combines disability level and duration of disease. In addition, SPMS patients were treated with a number of different DMTs, though there is no standard of care for SPMS (Rommer and Stuve (2013) Curr Treat Options Neurol 15(3):241-58). However, no evidence was found that molecular subgroups are affected by treatment.

These data indicate that patients with higher CTLA4-signature have higher chance of experiencing a relapse. CTLA-4 signaling plays a central role in the regulation of immune tolerance to self-antigens and control of cytotoxic T-cells (Romo-Tena, et al. (2013) Autoimmun Rev 12(12):1171-6). The findings associating the CTLA-4 pathway signature with more active disease and possibly a more aggressive course of RRMS, preceding clinically defined SPMS, provide support for targeting this biological pathway in RRMS. The observations indicate that targeting CTLA-4 pathway can be a beneficial for some RRMS patients, and, that molecular markers testing this pathway can be useful in identifying a suitable subpopulation. Alternatively, the CTLA-4 signature can be used more broadly to identify patients at higher risk of short-term disease worsening, in order to inform treatment decisions.

SPMS is also a heterogeneous population. In SPMS, most significant differences between the two subgroups are represented by B-cell signatures. This finding supports exploration of B-cell depleting therapies in SPMS. The identification of a subgroup of SPMS patients with higher expression of B-Cell signature suggests that B-cell depletion may not be an effective therapy for every SPMS patient, in which case molecular markers can help identify a suitable subpopulation.

### Materials and Methods

### Patients and Samples

Whole blood pre-treatment samples from 550 RRMS subjects (Table 1) assigned to the placebo arms of the DEFINE (NCT00420212) and CONFRIM (NCT00451451) (Fox, et al. (2012) N Engl J Med 367(12):1087-97; Gold, et al. (2012) N Engl J Med 367(12):1098-107) trials were selected for profiling; patients were either treatment-naïve or had not received disease-modifying therapy for at least 3 months (Gold, et al. (2012) N Engl J Med 367(12):1098-107). Prior to sample collection patients were treatment naïve for at least 3 months, and had been relapse-free for at least 3 months. Patients were followed over 2 years and disease severity was assessed every three months.

106 whole blood samples from patients diagnosed with SPMS (Table 2) were obtained from the Accelerated Cure Project (ACP) as an observational cohort, with only baseline data available. Patients had received a number of different MS treatments in the prior year as well as at the time of sample collection. Additional samples from 30 age and gender matched healthy controls were also provided by the ACP.

In both the SPMS and RRMS cohorts, the—MSSS—, which ranges from 0-10 and is a function of an individual's EDSS and time since MS onset, represents a spectrum of clinical disease severity (Roxburgh, et al. (2005) Neurology 64(7):1144-51). In RRMS cohort standardized neurologic assessments, including an EDSS, were performed every 3 months and at the time of suspected relapse (evaluated during unscheduled visits), however the SPMS cohort was assessed only at baseline. MSSS was chosen for this investigation because EDSS (Rudick, et al. (2010) Arch Neurol 67(11):1329-35), a commonly used clinical measure of disability, is not normally distributed and is thus less suited for the linear representation of molecular-clinical association. Additionally, MSSS captures longitudinal aspects of disease severity, while EDSS represents disability status.

### RNA Isolation, Labeling, Hybridization and Scanning

RNA was isolated from PaxGene tubes as per the manufacturer's standard protocol. RNA was quantified using the Nanodrop (Nanodrop Technologies, Willmington, Del.) and the quality was assessed using the Agilent 2100 Bioanalyzer (Agilent Technologies, Palo Alto, Calif.). 25 ng of Paxgene purified total RNA was amplified, fragmented and labeled using FL Ovation cDNA biotin automated module V2 (cat #A4200, Nugen Inc., San Carlos Calif.) and FL Ovation cDNA biotin automated module V2 (cat #A4200, Nugen inc., San Carlos Calif.) using Beckman Arrayplex automated workstation according to the manufacturer's protocol. 3 μg of fragmented and labeled cDNA was hybridized HGU132plus2/HTHGU-133plusPM arrays for RRMS/SPMS samples. Washing, staining and scanning of the hybridized arrays was completed as described in the Eukaryotic Target Preparation protocol in the Affymetrix expression analysis technical manual (702064 rev 2) for Genechip® cartridge arrays using the Genechip® Array Station (Affymetrix, Santa Clara, Calif.). Data was normalized using the GC-corrected Robust Multi-Array (GCRMA) method within SPMS/healthy controls and RRMS samples independently.

### Selection of Molecular Features

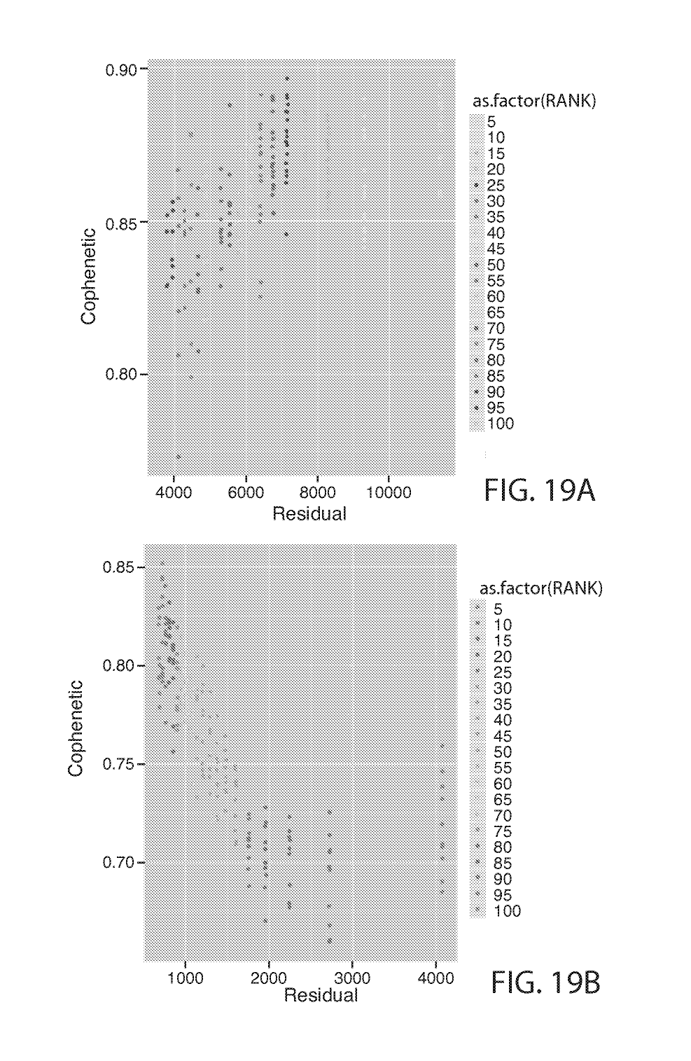

First, 106 SPMS profiles were compared to 30 profiles of gender and age matched healthy controls. 1753 transcripts that are significantly expressed in disease vs. controls were selected using the ProbeSelect method (Hosur, et al. (2014) Bioinformatics 30(4):574-5). Second, since gene expressions are correlated, the Nonnegative Matrix Factorization (NMF) (Lee and Seung (1999) Nature 401(6755):788-91) was applied to identify independent components among the 1753 transcripts. Using the cophenetic measure as a metric for evaluating the dimensionality reduction (Hofree, et al. (2013) Nat Methods 10(11):1108-15; Ottoboni, et al. (2012) Sci Transl Med 4(153):153ra131), 25 factors representing independent molecular signals were selected across the disease profiles. Cophenetic measure reaches plateau for 25 factors (FIG. 19A). For the RRMS profiles the same 1753 transcripts were used and similarly 20 factors were selected to represent molecular variability. Cophenetic measure reaches plateau for 20 factors (FIG. 19B). The 25/20 molecular factors were entered into the expertMIX algorithm together with MSSS values, for SPMS/RRMS cohorts.

### CTLA-4 Pathway Signature

11 genes (Zap70, PIK3R2, CD247, AKT2, NFATC2, LAT, LCK, FYN, CD3D, LAT, and TRAC) representing the CTLA-4 pathway, as defined in the Thomson Reuters MetaCore pathway database, were selected from 1753 transcripts. With hierarchical clustering of 11 genes' baseline expression, 3 stable subgroups of patients were identified. CTLA-4 pathway signature is then summarized as geometric mean of expression of these genes. FIG. 18A shows distributions of the CTLA4-siganture in the 3 clusters at baseline and after 1 and 2 years.

### Data Availability

Gene expression data for the RRMS cohort are available in Gene Expression Omnibus GEO (accession number). The anonymized SPMS patient and control data is available from the Accelerated Cure Project.

### Mixture of Expert Method

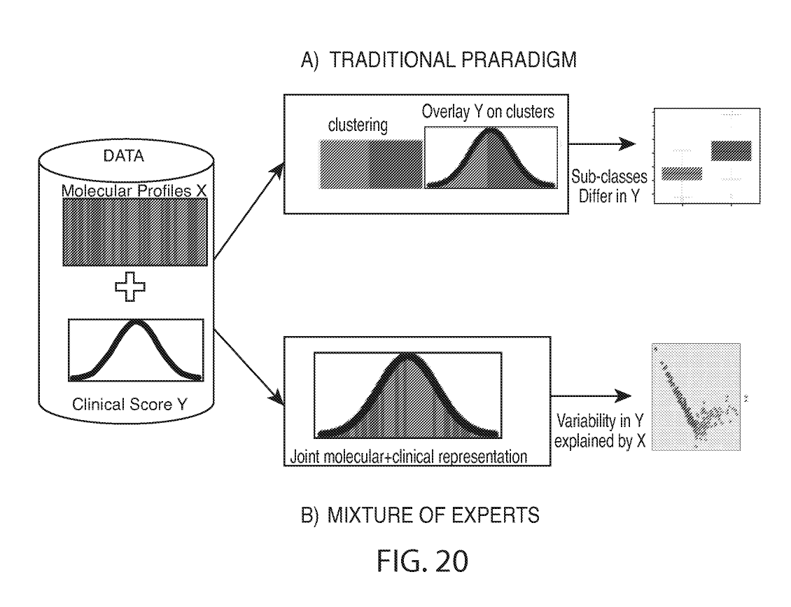

The approach using expertMIX stratification addresses the challenge of simultaneously discovering molecular variability that may underlie clinical heterogeneity. The conventional paradigm assumes that molecularly different patient samples might be associated with different clinical outcomes (Ottoboni, et al. (2012) Sci Transl Med 4(153):153ra131). In contrast, this method interrogates molecular characteristics that define uniform sample subsets and simultaneously an independent set of characteristics that may explain clinical heterogeneity. FIG. 20 illustrates differences between the method and the traditional approach.

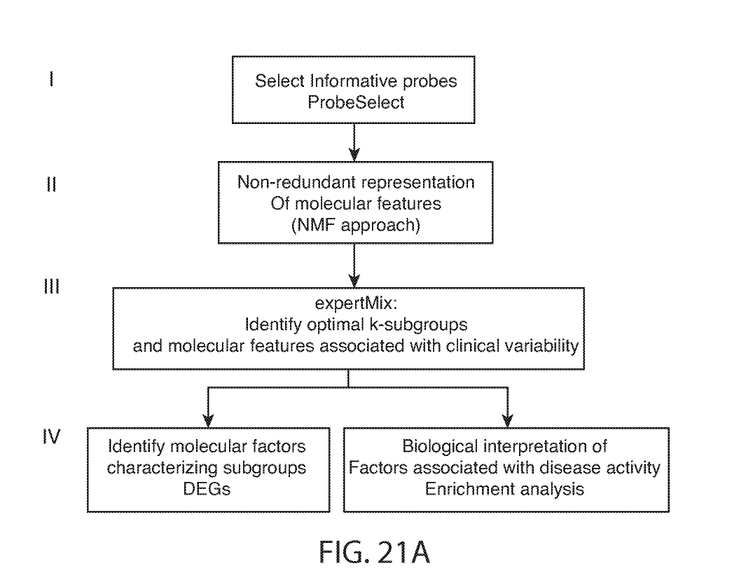

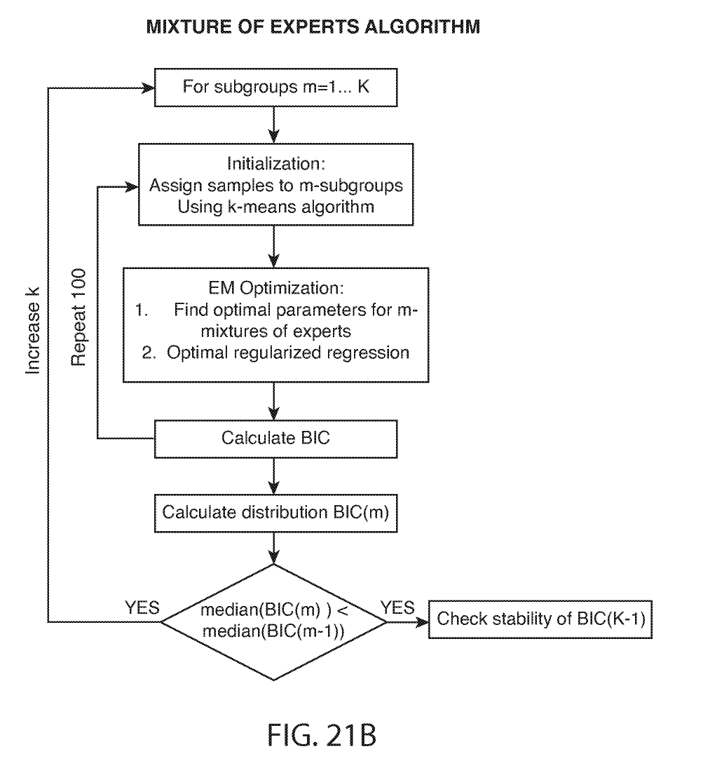

The ExpertMIX method is at the core of an approach to identify and interpret clinical and molecular variability. This approach follows 4 steps outlined in FIG. 21A: I) feature selection using ProbeSelect (Hosur, et al. (2014) Bioinformatics 30(4):574-5); II) identification of non-redundant feature representation, i.e. dimensionality reduction using Non-negative Matrix Factorization (NMF) (Lee and Seung (1999) Nature 401(6755):788-91); III) identification of molecular subgroups and features associated with clinical variability using expertMIX; and IV) molecular characterization of the subgroups and biological interpretation of features associated with clinical variability.

The outline of the expertMIX algorithm is shown in FIG. 21B. The algorithm explores the hypothesis that k=1, . . . , m subgroups of samples have different molecular characteristics associated with clinical heterogeneity. For each k the BIC (Bayesian Information Criterion) assesses the optimal mixture of expert models explaining disease variability in the entire set. Details of the optimization procedure for k-mixtures of experts are provided in Materials and Methods. Median BIC(k) is compared to the BIC(k−1) and the algorithm finds m=k such that the median(BIC(m))>median(BIC(m−1)). The distribution plots for k=1, . . . , m show stability of different mixture of experts models. Thus in the final model selection, the median BIC(k) and its variability can be considered.

The performance of the expertMIX method was also tested on simulated data.

### Biological Interpretation of the Factors

Each factor is interpreted in terms of enriched cell types using the DMAP data (Novershtern, et al. (2011) Cell 144(2):296-309). DMAP data is used to define signatures for each cell type. First, transcripts that are differentially expressed specifically in that cell type were selected. Each probe's contribution to a factor is then converted to a probability by normalizing over the factors. Probes that are specific to a factor have a higher probability. Next, a gene-set enrichment analysis (instead of correlation in the gene set enrichment analysis (GSEA) score, a constant of 1 is used) is carried out for each factor to determine enrichment of the cell type signatures. To assess the significance of the enrichment score permutation p-values for each cell type were calculated, and significant (p<0.05) cell types were selected as the interpretation of the factor.

### Transcripts Differentially Expressed Between Patient Subgroups

In order to identify transcripts defining molecular subgroups of patients standard differential expression analysis as implemented in the Bioconductor limma package was performed (Smyth (2004) Stat Appl Genet Mol Biol 3:Article3). Probes with p<0.05 after FDR correction and those that are at least 1.5-fold different between the groups as differentially expressed were selected.

### Pathway Signature Calculation

For a given set of genes that defined the pathway signature in the external database only those genes that are also considered as significantly up/down-regulated with respect to the healthy control population using ProbeSelect were selected (Hosur, et al. (2014) Bioinformatics 30(4):574-5). It was assumed that signature is composed of genes with correlated expression only and calculated a geometric mean over the genes in the signature for each sample. The geometric mean is more stable in the presence of outliers than arithmetic mean.

### EQUIVALENTS

Those skilled in the art will recognize, or be able to ascertain using no more than routine experimentation, many equivalents to the specific embodiments of the invention described herein. Such equivalents are intended to be encompassed.

What is claimed is:

1. A method of treating a subject having multiple sclerosis (MS), e.g., secondary-progressive multiple sclerosis (SPMS), or at risk of developing MS, e.g., SPMS, the method comprising:
administering an MS therapy, e.g., an MS therapy described herein, to a subject having MS, e.g., SPMS, or is at risk of developing M.S., e.g., SPMS,
wherein the subject has one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated.

2. The method of claim 1, comprising:
acquiring knowledge that a subject has one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated, and,
based upon that knowledge, administering the subject an MS therapy, e.g., an MS therapy described herein.

3. The method of claim 1, having one, two, or all of the followings:
wherein the gene associated with granulocytes is up-regulated, by at least about 0.5, 1, 2, 5, 10, 20, 50, 100, 200, 500, or 1000 fold, compared to a standard, e.g., an expression level of the gene in granulocytes in a normal subject;
wherein the gene associated with T cells is up-regulated, by at least about 0.5, 1, 2, 5, 10, 20, 50, 100, 200, 500, or 1000 fold, compared to a standard, e.g., an expression level of the gene in T cells in a normal subject; or
wherein the gene associated with erythrocytes is up-regulated, by at least about 0.5, 1, 2, 5, 10, 20, 50, 100, 200, 500, or 1000 fold, compared to a standard, e.g., an expression level of the gene in erythrocytes in a normal subject.

4. The method of claim 1, having one, two, or all of the following:
wherein the gene associated with granulocytes is a granulocyte-specific gene; wherein the gene associated with T cells is a T cell-specific gene; or wherein the gene associated with erythrocytes is an erythrocyte-specific gene.

5. The method of claim 1, wherein the two or more up-regulated genes are selected from two or more (e.g., 2, 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

6. The method of claim 1, wherein the MS therapy comprises one or more of the following: an anti-VLA-4 therapy, e.g., natalizumab; an anti-IL-2 receptor therapy, e.g., daclizumab; an interferon beta, e.g., interferon beta-1a or interferon beta-1b; a sphingosine 1-phosphate (SIP) antagonist, e.g., fingolimod; glatiramer acetate (GA); a CTLA-4 antagonist, e.g., an anti-CTLA antibody or a soluble CTLA-4 protein (e.g., Abatacept); or a CD20 antagonist, e.g., an anti-CD20 antibody (e.g., rituximab).

7. The method of claim 1, wherein the subject has been treated with an MS therapy, e.g., an alternative MS therapy.

8. The method of claim 1, further comprising acquiring a sample, e.g., a blood sample, from the subject.

9. The method of claim 1, further comprising determining one, two, or all of the following: the expression levels of two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample.

10. The method of claim 9, wherein the expression levels are determined prior to initiating, during, or after, a treatment in the subject.

11. The method of claim 9, wherein the expression levels are determined at the time of diagnosis of the subject with MS, e.g., SPMS or relapsing-remitting multiple sclerosis (RRMS).

12. The method of claim 9, wherein the expression levels of the genes are determined by a method described herein, e.g., oligonucleotide array or quantitative RT-PCR.

13. The method of claim 9, further comprising comparing the expression levels of the genes with a standard, e.g., expression levels of the same genes in the same cell type in a normal subject.

14. The method of claim 1, further comprising identifying a subject having one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated, for treatment with an MS therapy, e.g., an MS therapy described herein.

15. The method of claim 14, wherein the subject is already receiving an MS therapy, e.g., an MS therapy described herein, and the identification of the up-regulation of the genes indicates that the subject can receive an alternative MS therapy, e.g., an alternative MS therapy described herein.

16. The method of claim 14, wherein the subject is already receiving an MS therapy, e.g., an MS therapy described herein, and the identification of the up-regulation of the genes indicates that the subject should stop receiving the MS therapy, or the dose or dosing schedule of the MS therapy should be altered, e.g., reduced or increased.

17. The method of claim 1, further comprising identifying a clinical outcome (e.g., disease severity, disease progression, clinical outcome, or prognosis) of the subject having one or more of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, up-regulated, wherein the up-regulation is correlated with or indicative of a clinical score, e.g., a clinical score associated with disease severity, disease progression, clinical outcome, or prognosis, e.g., a clinical score described herein.

18. The method of claim 1, further comprising selecting an MS therapy, e.g., an MS therapy described herein, for the subject.

19. The method of claim 1, further comprising determining a clinical score or clinical marker for the subject, e.g., a clinical score or clinical marker associated with disease severity, disease progression, clinical outcome, or prognosis, e.g., a clinical score described herein, e.g., Expanded Disability Status Scale (EDSS), or Multiple Sclerosis Severity Score (MSSS), e.g., a clinical marker described herein, e.g., an MRI marker described herein.

20. The method of claim 1, further comprising selecting a subject having MS, e.g., SPMS, or at risk for MS, e.g., SPMS, for treatment with an MS therapy, e.g., an MS therapy described herein, based upon a determination of one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated.

21. The method of claim 1, wherein the subject has two or more genes in a pathway described herein, e.g., a CTLA-4 pathway, differentially expressed (e.g., down-regulated or up-regulated).

22. The method of claim 21, wherein the two or more genes in the pathway, e.g., the CTLA-4 pathway, are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

23. A method of treating and/or evaluating a subject having multiple sclerosis (MS), e.g., secondary-progressive multiple sclerosis (SPMS), or at risk of developing MS, e.g., SPMS, the method comprising:
administering an MS therapy, e.g., an MS therapy described herein, to a subject having MS, e.g., SPMS, or is at risk of developing SPMS, and
wherein the subject has one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated.

24. The method of claim 23, comprising:
acquiring knowledge that a subject has one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated, and,
based upon that knowledge, administering the subject an MS therapy, e.g., an MS therapy described herein.

25. The method of claim 23, having one or more of the following:
wherein the gene associated with granulocytes is down-regulated by at least about 10%, 20%, 30%, 40%, 50%, 60%, 70%, 80%, 90%, 95%, 99%, or 99.9%, compared to a standard, e.g., an expression level of the gene in granulocytes in a normal subject;
wherein the gene associated with T cells is down-regulated by at least about 10%, 20%, 30%, 40%, 50%, 60%, 70%, 80%, 90%, 95%, 99%, or 99.9%, compared to a standard, e.g., an expression level of the gene in T cells in a normal subject; or
wherein the gene associated with erythrocytes is down-regulated by at least about 10%, 20%, 30%, 40%, 50%, 60%, 70%, 80%, 90%, 95%, 99%, or 99.9%, compared to a standard, e.g., an expression level of the gene in erythrocytes in a normal subject.

26. The method of claim 23, having one, two, or all of the following:
wherein the gene associated with granulocytes is a granulocyte-specific gene; wherein the gene associated with T cells is a T cell-specific gene; or wherein the gene associated with erythrocytes is an erythrocyte-specific gene.

27. The method of claim 23, wherein the two or more down-regulated genes are selected from two or more (e.g., 2, 3, 4, 5, 6, 7, 8, 9, 10, 11, 12, 13, 14, or all) of FCRL1, IGHM, 231418_PM_at, CD22, IGH@, 217138_PM_x_at, POU2AF1, LOC283663, IGHM, MS4A1, IGL@, TCL1A, IGHD, CLLU1, or IGK@.

28. The method of claim 23, wherein the MS therapy comprises one or more of the following: an anti-VLA-4 therapy, e.g., natalizumab; an anti-IL-2 receptor therapy, e.g., daclizumab; an interferon beta, e.g., interferon beta-1a or interferon beta-1b; a sphingosine 1-phosphate (SIP) antagonist, e.g., fingolimod; glatiramer acetate (GA); a CTLA-4 antagonist, e.g., an anti-CTLA antibody or a soluble CTLA-4 protein (e.g., Abatacept); or a CD20 antagonist, e.g., an anti-CD20 antibody (e.g., rituximab).

29. The method of claim 23, wherein the subject has been treated with an MS therapy, e.g., an alternative MS therapy.

30. The method of claim 23, further comprising acquiring a sample, e.g., a blood sample, from the subject.

31. The method of claim 23, further comprising determining the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample.

32. The method of claim 31, wherein the expression levels are determined prior to initiating, during, or after, a treatment in the subject.

33. The method of claim 31, wherein the expression levels are determined at the time of diagnosis of the subject with MS, e.g., SPMS or relapsing-remitting multiple sclerosis (RRMS).

34. The method of claim 31, wherein the expression levels of the genes are determined by a method described herein, e.g., oligonucleotide array or quantitative RT-PCR.

35. The method of claim 31, further comprising comparing the expression levels of the genes with a standard, e.g., expression levels of the same genes in the same cell type in a normal subject.

36. The method of claim 23, further comprising identifying a subject having one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated, for treatment with an MS therapy, e.g., an MS therapy described herein.

37. The method of claim 36, wherein the subject is already receiving an MS therapy, e.g., an MS therapy described herein, and the identification of the down-regulation of the genes indicates that the subject can receive an alternative MS therapy, e.g., an alternative MS therapy described herein.

38. The method of claim 36, wherein the subject is already receiving an MS therapy, e.g., an MS therapy described herein, and the identification of the down-regulation of the genes indicates that the subject should stop receiving the MS therapy, or the dose or dosing schedule of the MS therapy should be altered, e.g., reduced or increased.

39. The method of claim 23, further comprising identifying a clinical outcome (e.g., disease severity, disease progression, clinical outcome, or prognosis) of the subject having one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated, wherein the down-regulation is correlated with or indicative of a clinical score, e.g., a clinical score associated with disease severity, disease progression, clinical outcome, or prognosis, e.g., a clinical score described herein.

40. The method of claim 23, further comprising selecting an MS therapy, e.g., an MS therapy described herein, for the subject.

41. The method of claim 23, further comprising determining a clinical score or clinical marker for the subject, e.g., a clinical score or clinical marker associated with disease severity, disease progression, clinical outcome, or prognosis, e.g., a clinical score described herein, e.g., Expanded Disability Status Scale (EDSS), or Multiple Sclerosis Severity Score (MSSS), e.g., a clinical marker described herein, e.g., an MRI marker described herein.

42. The method of claim 23, further comprising selecting a subject having MS, e.g., SPMS, or at risk for MS, e.g., SPMS, for treatment with an MS therapy, e.g., an MS therapy described herein, based upon a determination of one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated.

43. The method of claim 23, wherein the subject has two or more genes in a pathway described herein, e.g., a CTLA-4 pathway, differentially expressed (e.g., down-regulated or up-regulated).

44. The method of claim 43, wherein the two or more genes in the pathway, e.g., the CTLA-4 pathway, are selected from two or more (e.g., 2, 3, 4, 5, 6, 7, 8, 9, 10, or all) of Zap70, PIK3R2, CD247, AKT2, NFATC2, LAT, LCK, FYN, CD3D, LAT, and TRAC.

45. A method of treating a subject having multiple sclerosis (MS), e.g., secondary-progressive multiple sclerosis (SPMS), or at risk of developing MS, e.g., SPMS, the method comprising:
administering an MS therapy, e.g., an MS therapy described herein, to a subject,
wherein the subject has MS, e.g., SPMS, or is at risk of developing MS, e.g., SPMS, and wherein the subject has two or more genes described herein differentially expressed (e.g., up-regulated or down-regulated), e.g., two or more genes in a pathway described herein, e.g., a CTLA-4 pathway, differentially expressed (e.g., up-regulated or down-regulated).

46. The method of claim 45, wherein the two or more genes in the pathway, e.g., the CTLA-4 pathway, are selected from two or more (e.g., 2, 3, 4, 5, 6, 7, 8, 9, 10, or all) of Zap70, PIK3R2, CD247, AKT2, NFATC2, LAT, LCK, FYN, CD3D, LAT, and TRAC.

47. A method of identifying a subject having multiple sclerosis (MS), e.g., secondary-progressive multiple sclerosis (SPMS), or at risk of developing MS, e.g., SPMS, for treatment with an MS therapy, e.g., an MS therapy described herein, the method comprising:
providing a sample, e.g., a blood sample, from a subject having multiple sclerosis (MS), e.g., secondary-progressive multiple sclerosis (SPMS), or at risk of developing SPMS;
determining the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample;
comparing the expression levels of the genes with a standard, e.g., expression levels of the same genes in the same cell type in a normal subject; and
identifying the subject for treatment with an MS therapy, e.g., an MS therapy described herein, on the basis that the subject has one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated.

48. A method of identifying a subject having multiple sclerosis (MS), e.g., secondary-progressive multiple sclerosis (SPMS), or at risk of developing MS, e.g., SPMS, for treatment with an MS therapy, e.g., an MS therapy described herein, the method comprising:
providing a sample, e.g., a blood sample, from a subject having multiple sclerosis (MS), e.g., secondary-progressive multiple sclerosis (SPMS), or at risk of developing SPMS;
determining the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample;
comparing the expression levels of the genes with a standard, e.g., expression levels of the same genes in the same cell type in a normal subject; and
identifying the subject for treatment with an MS therapy, e.g., an MS therapy described herein, on the basis that the subject has one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated.

49. A method of treating or preventing one or more symptoms associated with multiple sclerosis (MS), e.g., secondary-progressive multiple sclerosis (SPMS), the method comprising:
administering an MS therapy, e.g., an MS therapy described herein, to a subject,
wherein the subject has MS, e.g., SPMS, or at risk of developing MS, e.g., SPMS, and
wherein the subject has one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated.

50. A method of treating or preventing one or more symptoms associated with multiple sclerosis (MS), e.g., secondary-progressive multiple sclerosis (SPMS), the method comprising:
administering an MS therapy, e.g., an MS therapy described herein, to a subject,
wherein the subject has MS, e.g., SPMS, or at risk of developing MS, e.g., SPMS, and
wherein the subject has one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated.

51. A method of evaluating or monitoring disease progression in a subject having multiple sclerosis (MS), e.g., secondary-progressive multiple sclerosis (SPMS), or at risk of developing MS, e.g., SPMS, the method comprising:
providing a sample, e.g., a blood sample, from a subject having multiple sclerosis (MS), e.g., secondary-progressive multiple sclerosis (SPMS), or at risk of developing MS, e.g., SPMS;
determining the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample;
comparing the expression levels of the genes with a standard, e.g., expression levels of the same genes in the same cell type in a normal subject; and
evaluating or monitoring disease progression on the basis that the subject has one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated.

52. A method of evaluating or monitoring disease progression in a subject having multiple sclerosis (MS), e.g., secondary-progressive multiple sclerosis (SPMS), or at risk of developing MS, e.g., SPMS, the method comprising:
providing a sample, e.g., a blood sample, from a subject having multiple sclerosis (MS), e.g., secondary-progressive multiple sclerosis (SPMS), or at risk of developing MS, e.g., SPMS;
determining the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample;
comparing the expression levels of the genes with a standard, e.g., expression levels of the same genes in the same cell type in a normal subject; and
evaluating or monitoring disease progression on the basis that the subject has one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated.

53. A method for generating a personalized multiple sclerosis (MS), e.g., secondary-progressive multiple sclerosis (SPMS), treatment report, the method comprising:
obtaining a sample, e.g., a blood sample, from a subject having MS, e.g., SPMS;
determining the expression levels of one or more of the following: two or more genes associated with granulocytes, two or more genes associated with T cells, or two or more genes associated with erythrocytes; and
selecting an MS therapy, e.g., an MS therapy described herein, based on the expression levels identified, up-regulation of one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated, indicates a first course of treatment; and
down-regulation of one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated, indicates a second different course of action.

54. A method for generating a personalized multiple sclerosis (MS), e.g., secondary-progressive multiple sclerosis (SPMS), treatment report, the method comprising:
obtaining a sample, e.g., a blood sample, from a subject having MS, e.g., SPMS;
determining the expression levels of one or more of the following: two or more genes associated with granulocytes, two or more genes associated with T cells, or two or more genes associated with erythrocytes, and
selecting an MS therapy, e.g., an MS therapy described herein, based on the expression levels identified, down-regulation of one, two, or all of the following: two or more (e.g., 2, 3, 4, 5, 6, 7, 8, 9, 10, 25, 50, 100, 250, 500, or more) genes associated with granulocytes down-regulated, two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with erythrocytes down-regulated, indicates a first course of treatment; and
up-regulation of one, two, or all of the following: two or more (e.g., 2, 3, 4, 5, 6, 7, 8, 9, 10, 25, 50, 100, 250, 500, or more) genes associated with granulocytes up-regulated, two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with erythrocytes up-regulated, indicates a second different course of action.

55. A method of determining a gene expression profile for a subject having multiple sclerosis (MS), e.g., secondary-progressive multiple sclerosis (SPMS), comprising:
directly acquiring knowledge of the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in a sample from a subject having MS, e.g., SPMS, and
responsive to a determination of down-regulation or up-regulation of the genes, one or more of:
(1) stratifying a subject population;
(2) identifying or selecting the subject as likely or unlikely to respond to an MS therapy, e.g., an MS therapy described herein;
(3) selecting an MS therapy, e.g., an MS therapy described herein;
(4) treating the subject; or
(5) prognosticating the time course and/or severity of the disease in the subject.

56. The method of claim 55, wherein responsive to the direct acquisition of knowledge of the expression levels of the genes, the subject is classified as a candidate to receive an MS therapy, e.g., an MS therapy described herein.

57. The method of claim 55 or 56, wherein responsive to the direct acquisition of knowledge of the expression levels of the genes, the subject is identified as likely to respond to an MS therapy, e.g., an MS therapy described herein.

58. The method of any of claims 47 to 55, wherein the subject has two or more genes described herein differentially expressed (e.g., up-regulated or down-regulated), e.g., two or more genes in a pathway described herein, e.g., a CTLA-4 pathway, differentially expressed (e.g., up-regulated or down-regulated).

59. A reaction mixture comprising:
a plurality of detection reagents, or one or more purified or isolated preparations thereof; and
a target nucleic acid preparation derived from a sample, e.g., a blood sample, from a subject having multiple sclerosis (MS), e.g., secondary-progressive multiple sclerosis (SPMS),
wherein said plurality of detection reagents can determine expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes.

60. A system for evaluating a subject population having multiple sclerosis (MS), e.g., secondary-progressive multiple sclerosis (SPMS), the system comprising at least one processor operatively connected to a memory, the at least one processor has:
a first plurality of values for a plurality of subjects having MS, e.g., SPMS, wherein each value is indicative of expression of a gene, e.g., a gene associated with granulocytes, T cells, or erythrocytes;
a second plurality of values for the plurality of subjects having MS, e.g., SPMS, wherein each value is indicative of a clinical score or clinical marker for a subject having MS, e.g., SPMS, e.g., a clinical score or clinical marker associated with disease severity, disease progression, clinical outcome, or prognosis, e.g., a clinical score described herein, e.g., Expanded Disability Status Scale (EDSS), or Multiple Sclerosis Severity Score (MSSS), e.g., a clinical marker described herein, e.g., an MRI marker described herein; and
a function that correlates the first plurality of values with the second plurality of values to provide an output of classification of the MS, e.g., SPMS, of the subject population.

61. The system of claim 60, wherein the correlative function determines the joint distribution of the plurality of the subjects in a space of gene expression (X) and clinical score (Y), e.g., by the likelihood maximization problem:
$$Θ = {arg max}_{Θ} P ( Y , X )$$
$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$
where Θ represents the set of parameters used to describe the joint distribution, which includes parameters for the linear regression used to describe P(Y|X,cₘ), parameters for describing the clusters P(X|cₘ) and P(cₘ).

62. The system of claim 61, wherein the correlative function uses a regularized Expectation-Maximization algorithm (EM) to learn a sparse set of parameters.

63. The system of claim 61, wherein the output indicates an optimal number of clusters for the subject population, e.g., using Bayesian information criterion (BIC).

64. A kit for identifying a subject having multiple sclerosis (MS), e.g., secondary-progressive multiple sclerosis (SPMS), or at risk of developing MS, e.g., SPMS, for treatment with an MS therapy, e.g., an MS therapy described herein, comprising tests for determining the expression levels of one, two, or all of the following: two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in a sample.

65. A method of treating a subject having multiple sclerosis (MS), e.g., relapsing-remitting multiple sclerosis (RRMS), or at risk of developing MS, e.g., secondary-progressive multiple sclerosis (SPMS), the method comprising:
administering an MS therapy, e.g., an MS therapy described herein, to a subject having RRMS, or is at risk of developing MS, e.g., SPMS, and
wherein the subject has one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated.

66. The method of claim 65, comprising:
acquiring knowledge that a subject has one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated, and,
based upon that knowledge, administering the subject an MS therapy, e.g., an MS therapy described herein.

67. The method of claim 65 or 66, having one or both of the followings:
wherein the gene associated with T cells is up-regulated, by at least about 0.5, 1, 2, 5, 10, 20, 50, 100, 200, 500, or 1000 fold, compared to a standard, e.g., an expression level of the gene in T cells in a normal subject, or
wherein the gene associated with granulocytes is up-regulated, by at least about 0.5, 1, 2, 5, 10, 20, 50, 100, 200, 500, or 1000 fold, compared to a standard, e.g., an expression level of the gene in granulocytes in a normal subject.

68. The method of claim 65, having one or both of the following:
wherein the gene associated with T cells is a T cell-specific gene; or wherein the gene associated with granulocytes is a granulocyte-specific gene.

69. The method of claim 65, wherein the two or more up-regulated genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

70. The method of claim 65, wherein the MS therapy comprises one or more of the following: an anti-VLA-4 therapy, e.g., natalizumab; an anti-IL-2 receptor therapy, e.g., daclizumab; an interferon beta, e.g., interferon beta-1a or interferon beta-1b; a sphingosine 1-phosphate (SIP) antagonist, e.g., fingolimod; glatiramer acetate (GA); a CTLA-4 antagonist, e.g., an anti-CTLA antibody or a soluble CTLA-4 protein (e.g., Abatacept); or a CD20 antagonist, e.g., an anti-CD20 antibody (e.g., rituximab).

71. The method of claim 65, wherein the subject has been treated with an MS therapy, e.g., an alternative MS therapy.

72. The method of claim 65, further comprising acquiring a sample, e.g., a blood sample, from the subject.

73. The method of claim 65, further comprising determining one or both of the following: the expression levels of two or more (e.g., 2, 3, 4, 5, 6, 7, 8, 9, 10, 25, 50, 100, 250, 500, or more) genes associated with granulocytes, two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with erythrocytes, in the sample.

74. The method of claim 73, wherein the expression levels are determined prior to initiating, during, or after, a treatment in the subject.

75. The method of claim 73 or 74, wherein the expression levels are determined at the time of diagnosis of the subject with MS, e.g., SPMS or RRMS.

76. The method of claim 73, wherein the expression levels of the genes are determined by a method described herein, e.g., oligonucleotide array or quantitative RT-PCR.

77. The method of claim 73, further comprising comparing the expression levels of the genes with a standard, e.g., expression levels of the same genes in the same cell type in a normal subject.

78. The method of claim 65, further comprising identifying a subject having one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated, for treatment with an MS therapy, e.g., an MS therapy described herein.

79. The method of claim 78, wherein the subject is already receiving an MS therapy, e.g., an MS therapy described herein, and the identification of the up-regulation of the genes indicates that the subject can receive an alternative MS therapy, e.g., an alternative MS therapy described herein.

80. The method of claim 78 or 79, wherein the subject is already receiving an MS therapy, e.g., an MS therapy described herein, and the identification of the up-regulation of the genes indicates that the subject should stop receiving the MS therapy, or the dose or dosing schedule of the MS therapy should be altered, e.g., reduced or increased.

81. The method of claim 65, further comprising identifying a clinical outcome (e.g., disease severity, disease progression, clinical outcome, or prognosis) of the subject having one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, up-regulated, wherein the up-regulation is correlated with or indicative of a clinical score, e.g., a clinical score associated with disease severity, disease progression, clinical outcome, or prognosis, e.g., a clinical score described herein.

82. The method of claim 65, further comprising selecting an MS therapy, e.g., an MS therapy described herein, for the subject.

83. The method of claim 65, further comprising determining a clinical score or clinical marker for the subject, e.g., a clinical score or clinical marker associated with disease severity, disease progression, clinical outcome, or prognosis, e.g., a clinical score described herein, e.g., Expanded Disability Status Scale (EDSS), or Multiple Sclerosis Severity Score (MSSS), e.g., a clinical marker described herein, e.g., an MRI marker described herein.

84. The method of claim 65, further comprising selecting a subject having MS, e.g., RRMS, or at risk for MS, e.g., SPMS, for treatment with an MS therapy, e.g., an MS therapy described herein, based upon a determination of one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated.

85. The method of claim 65, wherein the subject has two or more genes in a pathway described herein, e.g., a CTLA-4 pathway, differentially expressed (e.g., down-regulated or up-regulated).

86. The method of claim 85, wherein the two or more genes in the pathway, e.g., the CTLA-4 pathway, are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

87. A method of treating and/or evaluating a subject having multiple sclerosis (MS), e.g., relapsing remitting multiple sclerosis (RRMS), or at risk of developing MS, e.g., secondary-progressive multiple sclerosis (SPMS), the method comprising:
administering an MS therapy, e.g., an MS therapy described herein, to a subject,
wherein the subject has MS, e.g., RRMS, or is at risk of developing SPMS, and
wherein the subject has one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated.

88. The method of claim 87, comprising acquiring knowledge that a subject has one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated, and, based upon that knowledge, administering the subject an MS therapy, e.g., an MS therapy described herein.

89. The method of claim 87 or 88, having one or more of the following:
wherein the gene associated with T cells is down-regulated, by at least about 10%, 20%, 30%, 40%, 50%, 60%, 70%, 80%, 90%, 95%, 99%, or 99.9%, compared to a standard, e.g., an expression level of the gene in T cells in a normal subject, or
wherein the gene associated with granulocytes is down-regulated, by at least about 10%, 20%, 30%, 40%, 50%, 60%, 70%, 80%, 90%, 95%, 99%, or 99.9%, compared to a standard, e.g., an expression level of the gene in granulocytes in a normal subject.

90. The method of claim 87, having one or more of the following:
wherein the gene associated with T cells is a T cell-specific gene, or wherein the gene associated with granulocytes is a granulocyte-specific gene.

91. The method of claim 87, wherein the two or more down-regulated genes are selected from two or more (e.g., 3, 4, 5, 6, 7, 8, 9, 10, 11, 12, 13, 14, 15, 16, 17, 18, 19, or all) of TFEC, HLA-DQA1, MIR17HG, NMD3, PCGF5, MS4A1, KLHL14, RALGPS2, SESTD1, ZFP1, RIF1, FMNL2, USP25, NOC3L, IFT57, STK17B, MTF2, KMO, PTPRK, or CLLU1.

92. The method of claim 87, wherein the MS therapy comprises one or more of the following: an anti-VLA-4 therapy, e.g., natalizumab; an anti-IL-2 receptor therapy, e.g., daclizumab; an interferon beta, e.g., interferon beta-1a or interferon beta-1b; a sphingosine 1-phosphate (SIP) antagonist, e.g., fingolimod; glatiramer acetate (GA); a CTLA-4 antagonist, e.g., an anti-CTLA antibody or a soluble CTLA-4 protein (e.g., Abatacept); or a CD20 antagonist, e.g., an anti-CD20 antibody (e.g., rituximab).

93. The method of claim 87, wherein the subject has been treated with an MS therapy, e.g., an alternative MS therapy.

94. The method of claim 87, further comprising acquiring a sample, e.g., a blood sample, from the subject.

95. The method of claim 87, further comprising determining the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample.

96. The method of claim 95, wherein the expression levels are determined prior to initiating, during, or after, a treatment in the subject.

97. The method of claim 95 or 96, wherein the expression levels are determined at the time of diagnosis of the subject with MS, e.g., SPMS or RRMS.

98. The method of claim 95, wherein the expression levels of the genes are determined by a method described herein, e.g., oligonucleotide array or quantitative RT-PCR.

99. The method of claim 95, further comprising comparing the expression levels of the genes with a standard, e.g., expression levels of the same genes in the same cell type in a normal subject.

100. The method of claim 87, further comprising identifying a subject having one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated, for treatment with an MS therapy, e.g., an MS therapy described herein.

101. The method of claim 100, wherein the subject is already receiving an MS therapy, e.g., an MS therapy described herein, and the identification of the down-regulation of the genes indicates that the subject can receive an alternative MS therapy, e.g., an alternative MS therapy described herein.

102. The method of claim 100 or 101, wherein the subject is already receiving an MS therapy, e.g., an MS therapy described herein, and the identification of the down-regulation of the genes indicates that the subject should stop receiving the MS therapy, or the dose or dosing schedule of the MS therapy should be altered, e.g., reduced or increased.

103. The method of claim 87, further comprising identifying a clinical outcome (e.g., disease severity, disease progression, clinical outcome, or prognosis) of the subject having one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated, wherein the down-regulation is correlated with or indicative of a clinical score, e.g., a clinical score associated with disease severity, disease progression, clinical outcome, or prognosis, e.g., a clinical score described herein.

104. The method of claim 87, further comprising selecting an MS therapy, e.g., an MS therapy described herein, for the subject.

105. The method of claim 87, further comprising determining a clinical score or clinical marker for the subject, e.g., a clinical score or clinical marker associated with disease severity, disease progression, clinical outcome, or prognosis, e.g., a clinical score described herein, e.g., Expanded Disability Status Scale (EDSS), or Multiple Sclerosis Severity Score (MSSS), e.g., a clinical marker described herein, e.g., an MRI marker described herein.

106. The method of claim 87, further comprising selecting a subject having MS, e.g., SPMS, or at risk for MS, e.g., SPMS, for treatment with an MS therapy, e.g., an MS therapy described herein, based upon a determination of one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated.

107. The method of claim 87, wherein the subject has two or more genes in a pathway described herein, e.g., a CTLA-4 pathway, differentially expressed (e.g., down-regulated or up-regulated).

108. The method of claim 107, wherein the two or more genes in the pathway, e.g., the CTLA-4 pathway, are selected from two or more (e.g., 3, 4, 5, 6, 7, 8, 9, 10, or all) of Zap70, PIK3R2, CD247, AKT2, NFATC2, LAT, LCK, FYN, CD3D, LAT, and TRAC.

109. A method of treating a subject having multiple sclerosis (MS), e.g., relapsing-remitting multiple sclerosis (RRMS), or at risk of developing MS, e.g., secondary-progressive multiple sclerosis (SPMS), the method comprising:
administering an MS therapy, e.g., an MS therapy described herein, to a subject,
wherein the subject has MS, e.g., RRMS, or is at risk of developing MS, e.g., SPMS, and wherein the subject has two or more genes described herein differentially expressed (e.g., up-regulated or down-regulated), e.g., two or more genes in a pathway described herein, e.g., a CTLA-4 pathway, differentially expressed (e.g., up-regulated or down-regulated).

110. The method of claim 109, wherein the two or more genes in the pathway, e.g., the CTLA-4 pathway, are selected from two or more (e.g., (e.g., 3, 4, 5, 6, 7, 8, 9, 10, or all) of Zap70, PIK3R2, CD247, AKT2, NFATC2, LAT, LCK, FYN, CD3D, LAT, and TRAC.

111. A method of identifying a subject having multiple sclerosis (MS), e.g., relapsing-remitting multiple sclerosis (RRMS), or at risk of developing MS, e.g., secondary-progressive multiple sclerosis (SPMS), for treatment with an MS therapy, e.g., an MS therapy described herein, the method comprising:
providing a sample, e.g., a blood sample, from a subject having multiple sclerosis (MS), e.g., relapsing-remitting multiple sclerosis (RRMS), or at risk of developing SPMS;
determining the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample;
comparing the expression levels of the genes with a standard, e.g., expression levels of the same genes in the same cell type in a normal subject; and
identifying the subject for treatment with an MS therapy, e.g., an MS therapy described herein, on the basis that the subject has one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated.

112. A method of identifying a subject having multiple sclerosis (MS), e.g., relapsing-remitting multiple sclerosis (RRMS), or at risk of developing MS, e.g., SPMS, for treatment with an MS therapy, e.g., an MS therapy described herein, the method comprising:
providing a sample, e.g., a blood sample, from a subject having multiple sclerosis (MS), e.g., relapsing-remitting multiple sclerosis (RRMS), or at risk of developing SPMS;
determining the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample;
comparing the expression levels of the genes with a standard, e.g., expression levels of the same genes in the same cell type in a normal subject; and
identifying the subject for treatment with an MS therapy, e.g., an MS therapy described herein, on the basis that the subject has one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated.

113. A method of treating or preventing one or more symptoms associated with multiple sclerosis (MS), e.g., relapsing-remitting multiple sclerosis (RRMS), the method comprising:
administering an MS therapy, e.g., an MS therapy described herein, to a subject,
wherein the subject has MS, e.g., RRMS, or at risk of developing MS, e.g., SPMS, and
wherein the subject has one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated.

114. A method of treating or preventing one or more symptoms associated with multiple sclerosis (MS), e.g., relapsing-remitting multiple sclerosis (RRMS), the method comprising:
administering an MS therapy, e.g., an MS therapy described herein, to a subject,
wherein the subject has MS, e.g., RRMS, or at risk of developing MS, e.g., SPMS, and
wherein the subject has one or both of the following: two or more (e.g., 2, 3, 4, 5, 6, 7, 8, 9, 10, 25, 50, 100, 250, 500, or more) genes associated with T cells down-regulated, or wherein the subject has one or more of the following: two or more (e.g., 2, 3, 4, 5, 6, 7, 8, 9, 10, 25, 50, 100, 250, 500, or more) genes associated with granulocytes down-regulated.

115. A method of evaluating or monitoring disease progression in a subject having multiple sclerosis (MS), e.g., relapsing-remitting multiple sclerosis (RRMS), or at risk of developing MS, e.g., secondary-progressive multiple sclerosis (SPMS), the method comprising:
providing a sample, e.g., a blood sample, from a subject having multiple sclerosis (MS), e.g., relapsing-remitting multiple sclerosis (RRMS), or at risk of developing MS, e.g., SPMS;
determining the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample;
comparing the expression levels of the genes with a standard, e.g., expression levels of the same genes in the same cell type in a normal subject; and
evaluating or monitoring disease progression on the basis that the subject has one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated.

116. A method of evaluating or monitoring disease progression in a subject having multiple sclerosis (MS), e.g., relapsing-remitting multiple sclerosis (RRMS), or at risk of developing MS, e.g., secondary-progressive multiple sclerosis (SPMS), the method comprising:
providing a sample, e.g., a blood sample, from a subject having multiple sclerosis (MS), e.g., relapsing-remitting multiple sclerosis (RRMS), or at risk of developing MS, e.g., SPMS;
determining the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in the sample;
comparing the expression levels of the genes with a standard, e.g., expression levels of the same genes in the same cell type in a normal subject; and
evaluating or monitoring disease progression on the basis that the subject has one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated.

117. A method for generating a personalized multiple sclerosis (MS), e.g., relapsing-remitting multiple sclerosis (RRMS), treatment report, the method comprising:
obtaining a sample, e.g., a blood sample, from a subject having MS, e.g., SPMS;
determining the expression levels of one or more of the following: two or more genes associated with granulocytes, two or more genes associated with T cells, or two or more genes associated with erythrocytes, and
selecting an MS therapy, e.g., an MS therapy described herein, based on the expression levels identified, up-regulation of one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated, indicates a first course of treatment; and
down-regulation of one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated, indicates a second different course of action.

118. A method for generating a personalized multiple sclerosis (MS), e.g., relapsing-remitting multiple sclerosis (RRMS), treatment report, the method comprising:
obtaining a sample, e.g., a blood sample, from a subject having MS, e.g., SPMS;
determining the expression levels of one or both of the following: two or more genes associated with granulocytes, two or more genes associated with T cells, or two or more genes associated with erythrocytes, and
selecting an MS therapy, e.g., an MS therapy described herein, based on the expression levels identified, down-regulation of one or both of the following: two or more (e.g., 2, 3, 4, 5, 6, 7, 8, 9, 10, 25, 50, 100, 250, 500, or more) genes associated with T cells down-regulated, or two or more (e.g., 2, 3, 4, 5, 6, 7, 8, 9, 10, 25, 50, 100, 250, 500, or more) genes associated with granulocytes down-regulated, indicates a first course of treatment; and
up-regulation of one or both of the following: two or more (e.g., 2, 3, 4, 5, 6, 7, 8, 9, 10, 25, 50, 100, 250, 500, or more) genes associated with T cells up-regulated, or two or more (e.g., 2, 3, 4, 5, 6, 7, 8, 9, 10, 25, 50, 100, 250, 500, or more) genes associated with granulocytes up-regulated, indicates a second different course of action.

119. A method of determining a gene expression profile for a subject having multiple sclerosis (MS), e.g., relapsing-remitting multiple sclerosis (RRMS), comprising:
directly acquiring knowledge of the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in a sample from a subject having MS, e.g., SPMS, and
responsive to a determination of down-regulation or up-regulation of the genes, one or more of:
(1) stratifying a subject population;
(2) identifying or selecting the subject as likely or unlikely to respond to an MS therapy, e.g., an MS therapy described herein;
(3) selecting an MS therapy, e.g., an MS therapy described herein;
(4) treating the subject; or
(5) prognosticating the time course and/or severity of the disease in the subject.

120. The method of claim 119, wherein responsive to the direct acquisition of knowledge of the expression levels of the genes, the subject is classified as a candidate to receive an MS therapy, e.g., an MS therapy described herein.

121. The method of claim 119 or 120, wherein responsive to the direct acquisition of knowledge of the expression levels of the genes, the subject is identified as likely to respond to an MS therapy, e.g., an MS therapy described herein.

122. The method of any of claims 111 to 119, wherein the subject has two or more genes described herein differentially expressed (e.g., up-regulated or down-regulated), e.g., two or more genes in a pathway described herein, e.g., a CTLA-4 pathway, differentially expressed (e.g., up-regulated or down-regulated).

123. A reaction mixture comprising:
a plurality of detection reagents, or purified or isolated preparation thereof; and
a target nucleic acid preparation derived from a sample, e.g., a blood sample, from a subject having multiple sclerosis (MS), e.g., relapsing-remitting multiple sclerosis (RRMS),
wherein said plurality of detection reagents can determine expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes.

124. A system for evaluating a subject population having multiple sclerosis (MS), e.g., relapsing-remitting multiple sclerosis (RRMS), the system comprising at least one processor operatively connected to a memory, the at least one processor has:
a first plurality of values for a plurality of subjects having MS, e.g., RRMS, wherein each value is indicative of expression of a gene, e.g., a gene associated with T cells or granulocytes;
a second plurality of values for the plurality of subjects having MS, e.g., RRMS, wherein each value is indicative of a clinical score or clinical marker for a subject having MS, e.g., RRMS, e.g., a clinical score or clinical marker associated with disease severity, disease progression, clinical outcome, or prognosis, e.g., a clinical score described herein, e.g., Expanded Disability Status Scale (EDSS), or Multiple Sclerosis Severity Score (MSSS), e.g., a clinical marker described herein, e.g., an MRI marker described herein; and
a function that correlates the first plurality of values with the second plurality of values to provide an output of classification of the MS, e.g., RRMS, of the subject population.

125. The system of claim 124, wherein the correlative function determines the joint distribution of the plurality of the subjects in a space of gene expression (X) and clinical score (Y), e.g., by the likelihood maximization problem:
$$Θ = {arg max}_{Θ} P ( Y , X )$$
$$P ( Y , X ) = {\sum }_{m = 1}^{K} P ( Y , X , {c}_{m} ) = {\sum }_{m = 1}^{K} P ( Y \vert X , {c}_{m} ) P ( X \vert {c}_{m} ) P ( {c}_{m} )$$
where Θ represents the set of parameters used to describe the joint distribution, which includes parameters for the linear regression used to describe P(Y|X,cₘ), parameters for describing the clusters P(X|cₘ) and P(cₘ).

126. The system of claim 125, wherein the correlative function uses a regularized Expectation-Maximization algorithm (EM) to learn a sparse set of parameters.

127. The system of claim 125, wherein the output indicates an optimal number of clusters for the subject population, e.g., using Bayesian information criterion (BIC).

128. A kit for identifying a subject having multiple sclerosis (MS), e.g., relapsing-remitting multiple sclerosis (RRMS), or at risk of developing MS, e.g., secondary-progressive multiple sclerosis (SPMS), for treatment with an MS therapy, e.g., an MS therapy described herein, comprising tests for determining the expression levels of one or both of the following: two or more (e.g., 2, 3, 4, 5, 6, 7, 8, 9, 10, 25, 50, 100, 250, 500, or more) genes associated with T cells, or two or more (e.g., 2, 3, 4, 5, 6, 7, 8, 9, 10, 25, 50, 100, 250, 500, or more) genes associated with granulocytes, in a sample.
